SEQUENCE LISTING (63 sequences)

SEQ ID NO: 1 - DNA, 33 bp, Artificial Sequence
  primer
ggggtacccatggataaatttcgtgttcagggg

SEQ ID NO: 2 - DNA, 55 bp, Artificial Sequence
  primer
ccggaattctagattagtggtggtggtggtggtgttcgcctttcagacgctcaat

SEQ ID NO: 3 - DNA, 33 bp, Artificial Sequence
  primer
ggggtacccatggtcagtctgattgcggcgtta

SEQ ID NO: 4 - DNA, 56 bp, Artificial Sequence
  primer
cccaagcttctagattagtggtggtggtggtggtgccgccgctccagaatctcaaa

SEQ ID NO: 5 - DNA, 20 bp, Artificial Sequence
  primer
ggccatatcacctgtggtca

SEQ ID NO: 6 - DNA, 24 bp, Artificial Sequence
  primer
ccatttgtgccgcaacacgatagg

SEQ ID NO: 7 - DNA, 54 bp, Artificial Sequence
  primer
tgtgagcggataacaatttcacacaggaaaacaaaacgcggtcgcaccatcgca

SEQ ID NO: 8 - DNA, 20 bp, Artificial Sequence
  primer
attcgccgccagggtcaggc

SEQ ID NO: 9 - DNA, 24 bp, Artificial Sequence
  primer
cacagccgtggttgggaagataag

SEQ ID NO: 10 - DNA, 54 bp, Artificial Sequence
  primer
gtgcgtaacggcaaaagcaccgccggacatacactaagtttattggtgctgttc

SEQ ID NO: 11 - DNA, 20 bp, Artificial Sequence
  primer
atgccatagcatttttatcc

SEQ ID NO: 12 - DNA, 24 bp, Artificial Sequence
  primer
tctgatttaatctgtatcaggctg

SEQ ID NO: 13 - DNA, 54 bp, Artificial Sequence
  primer
tatgatgaacagcacccataaacttagtgtcgtctccgggagctgcatgtgtca

SEQ ID NO: 14 - DNA, 54 bp, Artificial Sequence
  primer
gcgtaatgcgatggtgcgaccgcgttttgtaactcacgttaagggattttggtc

SEQ ID NO: 15 - DNA, 24 bp, Artificial Sequence
  primer
gcgttaccattattgacccgaatg

SEQ ID NO: 16 - DNA, 54 bp, Artificial Sequence
  primer
tgtgagcggataacaatttcacacaggaaagcaaatattgagcgtgtgaaaggc

SEQ ID NO: 17 - DNA, 24 bp, Artificial Sequence
  primer
ctgctcaacgtgaagcctactttg

SEQ ID NO: 18 - DNA, 24 bp, Artificial Sequence
  primer
ctacatgcgcgttatctatagcaa

SEQ ID NO: 19 - DNA, 54 bp, Artificial Sequence
  primer
gtgcgtaacggcaaaagcaccgccggacatcgttggcccctgaacacgaaattt

SEQ ID NO: 20 - DNA, 24 bp, Artificial Sequence
  primer
catgtcagaagccgtaaacgtcat

SEQ ID NO: 21 - DNA, 24 bp, Artificial Sequence
  primer
tgttagggcagggcagtgagtttg

SEQ ID NO: 22 - DNA, 54 bp, Artificial Sequence
  primer
tgtgagcggataacaatttcacacaggaaatattgctttgagattctggagcgg

SEQ ID NO: 23 - DNA, 24 bp, Artificial Sequence
  primer
ctcatcatcaaaatcgccatgctg

SEQ ID NO: 24 - DNA, 24 bp, Artificial Sequence
  primer
ccatgcagggtcactacgaaatga

SEQ ID NO: 25 - DNA, 54 bp, Artificial Sequence
  primer
gtgcgtaacggcaaaagcaccgccggacattaccgctaacgccgcaatcagact

SEQ ID NO: 26 - DNA, 24 bp, Artificial Sequence
  primer
ataccgtaaccatacgcaatctgg

SEQ ID NO: 27 - DNA, 50 bp, Artificial Sequence
  primer
atgtccggcggtgcttttgccgttacgcacaagccacgttgtgtctcaaa

SEQ ID NO: 28 - DNA, 20 bp, Artificial Sequence
  primer
gaaattgttatccgctcaca

SEQ ID NO: 29 - DNA, 24 bp, Artificial Sequence
  primer
attggttgtaacactggcagagca

SEQ ID NO: 30 - DNA, 24 bp, Artificial Sequence
  primer
atcatcaggagtacggataaaatg

SEQ ID NO: 31 - DNA, 62 bp, Artificial Sequence
  primer
gtacgcagtgatgtttccagcatttccgacgcggccgcggagctgcatgtgtcagaggtttt

SEQ ID NO: 32 - DNA, 54 bp, Artificial Sequence
  primer
agccccacgaaggaggtgaggaagtagaagaaacgaggtcatcatttccttccg

SEQ ID NO: 33 - DNA, 54 bp, Artificial Sequence
  primer
cttctacttcctcacctccttcgtggggtctacacagcccagtccagactattc

SEQ ID NO: 34 - DNA, 64 bp, Artificial Sequence
  primer
tttgagtgcctccttataatttattttgtagttccttcgaagcttaattgttatccgctcacaa

SEQ ID NO: 35 - DNA, 65 bp, Artificial Sequence
  primer
tcgaaggaactacaaaataaattataaggaggcactcaaattggaaaaaatcatcgtccgcggcg

SEQ ID NO: 36 - DNA, 63 bp, Artificial Sequence
  primer
ttattgtcctgggtttcaagcattagttccaggccggcctgccagagtgacatactgattttc

SEQ ID NO: 37 - DNA, 57 bp, Artificial Sequence
  primer
tttggcgcgcctttggccggcctttgcggccgcgtcggaaatgctggaaacatcact

SEQ ID NO: 38 - DNA, 57 bp, Artificial Sequence
  primer
tttggccggcctttggcgcgcctaattaattaatgcggtaagggttgactgagttga

SEQ ID NO: 39 - DNA, 54 bp, Artificial Sequence
  primer
cccttgtcaactcagtcaacccttaccgcaataagcttgatgaaaatttgtttg

SEQ ID NO: 40 - DNA, 54 bp, Artificial Sequence
  primer
gtacgcagtgatgtttccagcatttccgacggggcaggttagtgacattagaaa

SEQ ID NO: 41 - DNA, 24 bp, Artificial Sequence
  primer
aattccggcgactgtttctgtttc

SEQ ID NO: 42 - DNA, 24 bp, Artificial Sequence
  primer
catgtaaaagatgaggttggttca

SEQ ID NO: 43 - DNA, 24 bp, Artificial Sequence
  primer
attcacatttgacagctcctttcc

SEQ ID NO: 44 - DNA, 55 bp, Artificial Sequence
  primer
taaaaatcaaacaaattttcatcaagcttattttgactgtgccgtttaacttctg

SEQ ID NO: 45 - DNA, 56 bp, Artificial Sequence
  primer
agtcggttttctaatgtcactaacctgcccccgagcaagagaataaagaagtcgtt

SEQ ID NO: 46 - DNA, 24 bp, Artificial Sequence
  primer
cctatgaaaagagaagattgtccg

SEQ ID NO: 47 - DNA, 24 bp, Artificial Sequence
  primer
ctcaccatattgcttttcaggctg

SEQ ID NO: 48 - DNA, 24 bp, Artificial Sequence
  primer
tgatttccacctaacgcccctatg

SEQ ID NO: 49 - DNA, 24 bp, Artificial Sequence
  primer
ggggcaggttagtgacattagaaa

SEQ ID NO: 50 - DNA, 24 bp, Artificial Sequence
  primer
ataagcttgatgaaaatttgtttg

SEQ ID NO: 51 - DNA, 16 bp, Artificial Sequence
  primer
ngtcttcaccacgggg

SEQ ID NO: 52 - DNA, 11 bp, Artificial Sequence
  primer
gtggtgaagac

SEQ ID NO: 53 - DNA, 12 bp, Artificial Sequence
  primer
ccagccccttgg

SEQ ID NO: 54 - DNA, 12 bp, Artificial Sequence
  primer
ccaaggggctgg

SEQ ID NO: 55 - DNA, 20 bp, Artificial Sequence
  primer
gcgcatcagcatcgtggaat

SEQ ID NO: 56 - DNA, 20 bp, Artificial Sequence
  primer
agtgcggcaaaaaggatagg

SEQ ID NO: 57 - DNA, 20 bp, Artificial Sequence
  primer
aaagggtcagcagggcagaa

SEQ ID NO: 58 - DNA, 20 bp, Artificial Sequence
  primer
cgcgtcagggtaataaatgg

SEQ ID NO: 59 - DNA, 20 bp, Artificial Sequence
  primer
gcagggtcggaacaggagag

SEQ ID NO: 60 - DNA, 48 bp, Artificial Sequence
  primer
gtcgtcgtgtcgacgctcttcctaaccatggtctctgatatctaacta

SEQ ID NO: 61 - DNA, 48 bp, Artificial Sequence
  primer
acgacgacgtcgacattgaagagccctccttatctagatttttgtcga

SEQ ID NO: 62 - DNA, 200 bp, Artificial Sequence
  primer
ccatcgaatggccagatgattaattcctaatttttgttgacactctatcattgatagagttattttaccactccctatcagtgatagagaaaagtgaaatgaatagttcgacaaaaatctagataaggagggctcttcaatgtcgacgctcttcctaaccatggtctctgatatctaactaagcttgacctgtgaagtga

SEQ ID NO: 63 - DNA, 200 bp, Artificial Sequence
  primer
ggtagcttaccggtctactaattaaggattaaaaacaactgtgagatagtaactatctcaataaaatggtgagggatagtcactatctcttttcactttacttatcaagctgtttttagatctattcctcccgagaagttacagctgcgagaaggattggtaccagagactatagattgattcgaactggacacttcact

(19) United States
(12) Patent Application Publication
(10) Pub. No.: US 2003/0108872 A1
(43) Pub. Date: Jun. 12, 2003

(54) Genomics-assisted rapid identification of targets

(76) Inventors: Mark Sulavik, Holliston, MA (US); Losee Lucy Ling, Arlington, MA (US); Tim Opperman, Sommerville, MA (US); Donald T. Moir, Lexington, MA (US); Christopher Bunker, Sommerville, MA (US)

Correspondence Address: BURNS, DOANE, SWECKER & MATHIS, L.L.P., P.O. Box 1404, Alexandria, VA, 22313-1404, US

(21) Appl. No.: 09/934,489

(22) Filed: Aug. 23, 2001

Related U.S. Application Data
(60) Provisional application No. 60/226,896, filed on Aug. 23, 2000.

(51) Int. Cl. C12Q 1/68; G01N 33/48

(52) U.S. Cl. 435/6; 702/19

(57) ABSTRACT

The invention relates to methods and systems of determining targets of compounds and compositions, as well as a high through-put method of screening antimicrobial agents. The methods involve (i) the prediction of a target using two and preferably at least three different submethods, (ii) identifying the gene encoding the target by each of these submethods, (iii) analysing the potential targets of each submethod to predict the putative target, and (iv) validating the target and the drug which perturbs it.

### FIELD OF THE INVENTION

A method employing several molecular genetic and functional genomic techniques to identify the molecular target and/or mode of action of drugs. Preferably, at least three methods are used to determine the target of a compound. The methods and kits of the invention are generally applicable to identifying the molecular targets of compounds, such as antimicrobial or antifungal agents active against Gram positive and/or Gram negative bacteria or against fungi.

### BACKGROUND OF THE INVENTION

Almost all classes of antibacterial therapeutics on the market today were found initially by screening whole microbial cells for active compounds which inhibit growth and/or viability of the cells. The main advantage to screening whole cells is the efficiency. In essence, all the essential genes are screened at one time. These assays also deal simultaneously with issues of potency and cell penetration by active compounds. The main disadvantage of these types of assays is that the target and mechanism of action of resulting hits are unknown. While whole cell screening has been in use for decades, in recent years, whole-cell screening has failed to deliver, creating some doubts about the continued utility of this approach. Since the main problem with hits found by whole cell screening is absence of knowledge about the target and the risk that the mechanism of action is non-specific to microbes, availability of an effective technology for identifying the target would revitalize this approach.

Additionally, despite the development of numerous antibacterial agents, bacterial infections along with conditions regulated by other pathogens continue as a major and currently increasing medical problem. For instance, prior to the 1980's, bacterial infections in developed countries could be readily treated with available antibiotics. However, during the 1980's and 1990's, antibiotic resistant bacterial strains emerged and have become a major health problem. There are in fact, strains resistant to essentially all of the commonly used antibacterial agents which have been observed in the clinical setting, notably including strains of Staphylococcus aureus. The consequences of the increase in resistant strains include higher morbidity and mortality, longer patient hospitalization, and an increase in treatment costs (Murray, 1994, New Engl. J. Med. 330: 1229-30). Therefore, there is a pressing need for the development of new antibacterial and antimicrobial agents which are not significantly affected by the existing bacterial and pathogen resistance mechanisms. Such development of new antimicrobial agents can proceed by a variety of methods, but generally fall into at least two categories. The first is the traditional approach of screening for antibacterial agents without concern for the specific gene or protein target.

In contrast, target-based screening provides knowledge relating to a particular molecular target because screens are built from known targets. However, these approaches have some limitations which reduce their utility. While more cellular targets are available because of recent DNA sequencing efforts applied to bacterial and fungal genomes, many cannot be formatted readily in biochemical assays for screening. These could be screened in cell-based target-specific assays such as overexpression rescue assays or underexpression hypersusceptibility assays, but it is difficult to prioritize the thousands of cellular targets to arrive at a high quality, manageable list. Furthermore, not all targets are equally “drugable”, or in other words, equally capable of being inhibited by small molecule drugs. Therefore, considerable time can be wasted in target-based screening, focusing on targets which will rarely yield useful compounds. Finally, while biochemical target-based assays may yield inhibitory compounds, it is virtually impossible to predict whether the compounds can gain access to the target within the whole cell. If the compounds derived from biochemical target-based screens show no whole-cell activity, chemical modifications may succeed in optimizing this feature, but success is not guaranteed and typically is in the 50% range. Therefore, considerable time can be wasted identifying compounds which inhibit a biochemical screen but can not be optimized into useful antimicrobials.

This invention provides a method to reap the benefits of both cell-based and target-based screening, but without most of the disadvantages. This invention may be considered “reversed target-based screening” because it relies first on identification of compounds with good whole cell activity and then provides a set of methods to identify the targets of those compounds. The invention consists of preferably at least three methods, which when used together are effective for identifying the cellular target for most compounds with whole cell activity. Any of the methods of the invention may not be successful in isolation, but in combination of at least two or more (and preferably at least three or more) provides a high likelihood of success in identifying the molecular target.

### SUMMARY OF THE INVENTION

One object of the invention is to provide a method of identifying the molecular target of an inhibitory compound comprising the steps of: exposing a number of cells to said inhibitory compound; identifying a number of genes or gene products that are modulated by the presence of the inhibitory compound, in accordance with each of a plurality of target prediction processes; comparing the genes or gene products identified by each of the plurality of target prediction processes with the genes or gene products identified by each of the other target prediction processes; based on said comparison, selecting a gene or gene product from amongst the number of genes or gene products that were identified by each of the plurality of target prediction processes, and establishing the molecular target, wherein said molecular target is the selected gene product or a gene product associated with said selected gene. The molecular target is preferably protein or mRNA.

Another object of the invention described is to provide a method of identifying the molecular target of an inhibitory compound comprising the steps of: identifying a number of genes or gene products associated with a first population of cells, in accordance with each of a plurality of target prediction processes, wherein said genes or gene products are functionally modulated by the presence of the inhibitory compound; selecting, from amongst the number of genes or gene products, a first gene or gene product; in a second population of cells, down regulating the selected first gene, or a gene associated with the selected first gene product, through a regulatable promoter; and determining whether the selected first gene product, or a gene product associated with the selected first gene, is a valid molecular target of the inhibitory compound based on characteristics that are associated with the second population of cells.

Preferred cells and organisms contemplated by the invention are bacteria and fungi, but other prokaryotes and eukaryotes are also contemplated.

It is a further object of the invention to identify compounds and compositions which target the genes identified by the above methods. Compounds and compositions thus identified are contemplated for use in administering to a subject afflicted with a condition or disease caused by an organism in which said gene is located.

### BRIEF DESCRIPTION OF THE DRAWINGS

FIG. 1 shows the overall schema for under-expression of genes in E. coil and B. subtilis.

FIG. 2 shows the schema to generate universal cloning templates to insert a regulatable gene into the yibD locus.

FIG. 3 shows that for cells in which folA is regulated by P_{BAD}, P_{LtetO-1}, or P_{lac/ara-1}, the MIC of trimethoprim decreases with decreasing concentrations of regulators (e.g. L-arabinose, anhydrotetracycline, and IPTG, respectively), whereas MIC of phosphomycin is relatively unchanged.

FIG. 4 shows that for cells in which murA is regulated by P_{BAD}, the MIC of phosphomycin decreases with decreasing regulator (L-arabinose) concentrations, whereas the MIC of trimethoprim is relatively unchanged.

FIG. 5 shows that for cells in which folA is regulated by P_{BAD}, the MIC of both trimethoprim and sulfamethozasone decreases with decreasing regulator (L-arabinose) levels. Trimethoprim and sulfamethozasone are two different antibiotics that act at different steps of the folate biosynthesis pathway.

FIG. 6 describes murA regulation by P_{BAD}. Results show that under-expression of mura leads to hypersensitivity to both phosphomycin and D-cycloserine. Phosphomycin and D-cylcoserine are drugs that act in the same pathway. The cells exposed to trimethoprim lactate represents the positive control wherein the under-expressed gene is not sensitive to the administration of trimethoprim lactate.

FIG. 7. Strategy to reconfigure Pspac expression system using crossover PCR.

FIG. 8. Strategy for construction of Pspac ectopic expression system.

FIG. 9. Two-step strategy for ectopic expression strain construction. FIG. 9A. Introduction of ectopic expression construct into ThrC locus of recipient strain by homologous recombination. FIG. 9B shows the strategy to delete the endogenous gene.

FIG. 10. Under expression of murA by Pspac-murA ectopic expression construct leads to increased sensitivity to phosphomycin.

FIG. 11. View of phosphomycin induced gene set revealed by hybridization of a microarray of S. epidermidis genes with differentially labeled cDNA derived from total RNA from S. epidermidis cells grown in the absence of phosphomycin in exponential phase and from cells grown in the presence of moderately inhibiting concentrations of phosphomycin. Differential gene expression profiles are also displayed for microorganisms grown in the absence of drug versus in the presence of ampicillin (Amp), no drug (control), cycloserine, erythromycin, vancomycin, and in the absence of drug but in the “lag” or “stationary” phase of growth.

FIG. 12. Flowchart of general GARIT technique.

FIG. 13. FIG. 13A is an example of a potential outcome produced using the GARIT technique. FIG. 13B is an example of genes predicted using the GARIT technique 100, wherein the Transformation Selection Method 110 does not predict any gene. FIG. 13C is an example of a potential outcome produced wherein the cell to which the agent has been administered has a response involving efflux pumps or drug modification enzymes. FIG. 13D is an example of an outcome using a GARIT technique 100, wherein at least two genes are predicted by the independent target prediction processes 110-125.

FIG. 14. Flowchart example of the Validation portion of the GARIT process

wherein more than one gene is targeted by a particular compound or composition.

FIG. 15. Overexpression of folA or murA genes on plasmids rescues E. coli cells from killing by trimethoprim or phosphomycin, respectively.

FIG. 16. Nucleotide sequence (nucleotides 1-200) of the ORFmer cloning site of pHO/0003.

FIG. 17. Results of an Overexpression Rescue (OER) assay performed in a liquid microtiter format using the following antibiotics with known molecular targets.

FIG. 18. Results of an OER assay performed in two plate formats using the following antibiotics with known molecular targets.

FIG. 19. Results of PCR verification of clones exhibiting OER in three assay formats for antibiotics with known molecular targets.

### DETAILED DESCRIPTION OF THE INVENTION

This invention is directed towards target based screening of cells and organisms to identify agents which modulate those cells as well to identify essential genes of the cell. The cells include both prokaryotic cells, as well as eukaryotic cells. Preferred cells are microorganisms (e.g., bacteria, amebas, fungi, protozoans, etc.), but can include human cells (e.g., malignant cells). The target based screening is directed toward a gene or genes necessary to the survival of pathogens.

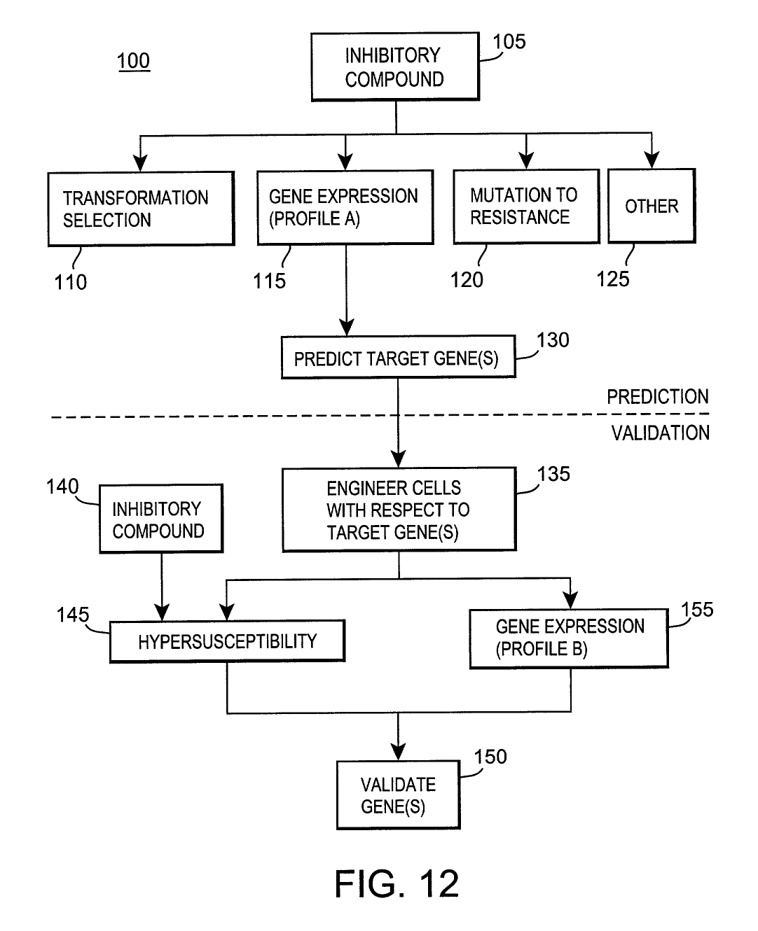

Generally, GARIT can be summarized as follows. FIG. 12 illustrates a technique 100 for identifying molecular targets, associated with a given anti-microbial agent in accordance with a preferred embodiment of the present invention, wherein anti-microbial agents include, but are not limited to, chemicals, chemical compounds, drugs, and herbal extracts. This technique is particularly useful in testing and/or developing new drugs (e.g., antibiotics).

As indicated in FIG. 12, the technique 100 is divided into a target prediction phase and a target validation phase. During the target prediction phase, cells (e.g., E. coli or B. subtilis) are exposed to an inhibitory compound or anti-microbial agent, as shown by step 105. Then, in accordance with each of a number of independent target prediction processes 110-125, prospective molecular targets are identified, wherein a prospective molecular target is one that is essential to cell survival. For the purpose of illustration and not limitation, three specific target prediction processes are presented in FIG. 12: a transformation selection process 110, a gene expression process 115, and a mutation to resistance process 120. Other target prediction processes, as shown by step 125, include Y3H, metabolic profiling, and proteomic profiling. Although, each of the aforementioned target prediction processes are individually known in the art, a detailed description of each is presented below in order to facilitate a better understanding of the target identification technique described herein. For sake of discussion, the term target or molecular target refers to a gene product (e.g., RNA or proteins) identified by one of the aforementioned target prediction processes, or a gene product associated with a gene identified by one of the target prediction processes.

Referring now to step 130, the results associated with each target prediction process 110-125 (i.e., the various genes identified by each of the independent target prediction processes) are correlated and analyzed to determine whether two or more of the target prediction processes 110-125 identify the same molecular target or gene encoding a particular molecular target. Accordingly, a molecular target that is identified by two or more of the target prediction processes 110-125 is identified as a predicted target. As one skilled in the art will readily appreciate, a weighting factor may be applied to the result associated with each target prediction process 110-125. Weighting factors might be useful in identifying targets when the results associated with two or more of the target prediction processes 110-125 are not wholly consistent with one another. Weighting factors might also be useful when certain target prediction processes 110-125 more accurately identify prospective targets than other target prediction processes, and greater deference towards the results associated with these processes is desirable.

As stated previously, the technique 100 illustrated in FIG. 12 is divided into a prediction phase and a validation phase. During the validation phase, the predicted target or targets identified during the prediction phase are now validated. In accordance with a preferred embodiment of the present invention, validation first involves engineering additional cells, as shown by step 135, such that the cells contain a given gene encoding the predicted target (e.g., protein) at its normal locus L₁, and an engineered version of the gene at a second locus L₂. Then, through the use of a promoter, the engineered version of the gene or nucleic acid at locus L₂ is down-regulated, while the gene at normal locus L₁ is deleted or disrupted. The susceptibility, or more particularly, the hypersusceptibility of the engineered cells in the presence of the inhibitory compound is then determined, as shown by steps 140 and 145. The result associated with the hypersusceptibility process may then be used to determine whether the predicted molecular target is a valid target of the inhibitory compound, in accordance with step 150. Alternatively, or in addition to the hypersusceptibility process, a gene profile (i.e., profile B) is generated from the microarray associated with the gene expression process, as shown by step 155. A comparison between gene expression profile B and gene expression profile A, which was generated in accordance with step 115, may then be used to determine whether the predicted molecular target is a valid target of the inhibitory compound, again, in accordance with step 150.

The hypersusceptibility process, as shown by step 145, involves, more specifically, varying the degree to which the engineered gene encoding the target at locus L₂ is down-regulated. This may be accomplished by exposing separate populations of engineered cells to different concentrations of a promoter regulatable agent, wherein greater concentrations of the promoter regulatable agent increase the down-regulation level of the engineered gene. Each of the cell populations is then exposed to the inhibitory compound, as shown by step 140. If, in analyzing the different cell populations, it appears that cell susceptibility increases proportionally with the down-regulation of the engineered gene, this tends to indicate that the gene product associated with this gene is, in fact, a target of the inhibitory compound.

The second gene expression profiling procedure, as shown by step 155, involves, more specifically, the creation of a microarray based on the cells that were engineered in accordance with step 135. The microarray, in turn, produces a corresponding profile, which is shown as profile B in FIG. 12, wherein profile B identifies those genes that are up-regulated or down-regulated as a result of the down-regulation of the engineered gene at locus L₂. Profile B, as stated above, is then compared to profile A, and if profile B substantially matches profile A, this tends to validate the fact that the gene product associated with the engineered gene is a target of the inhibitory compound.

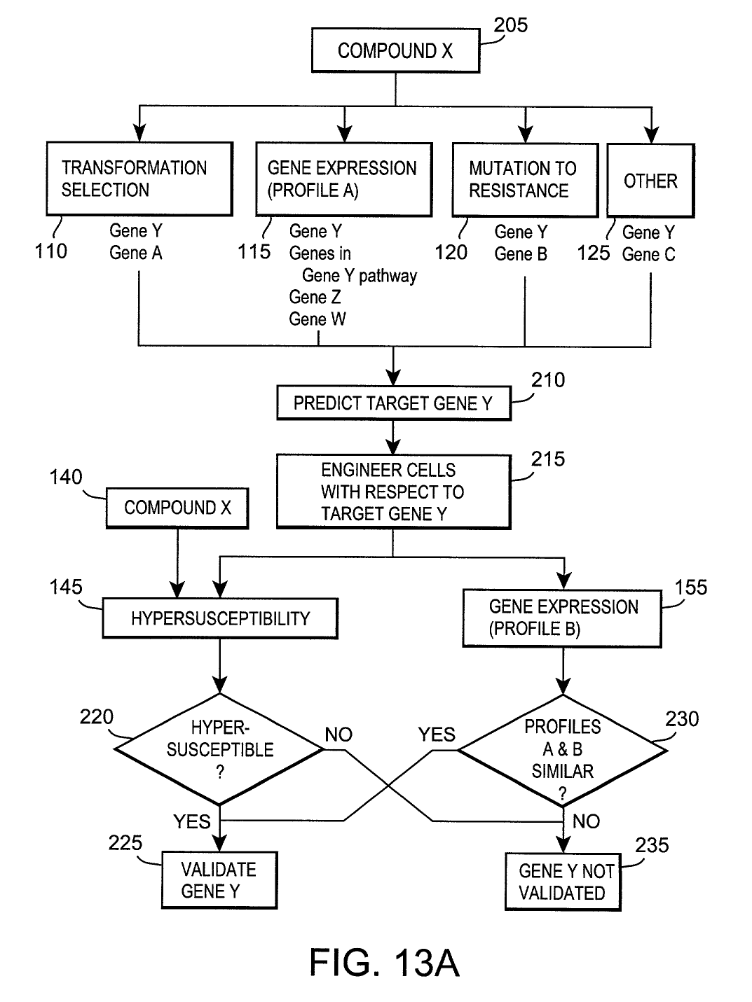

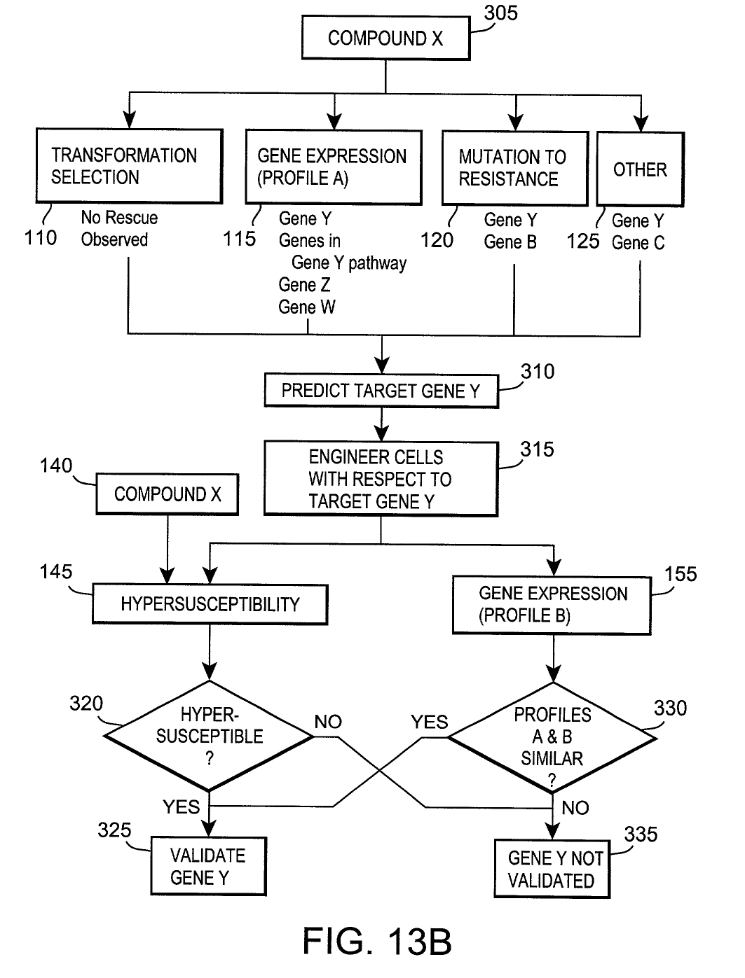

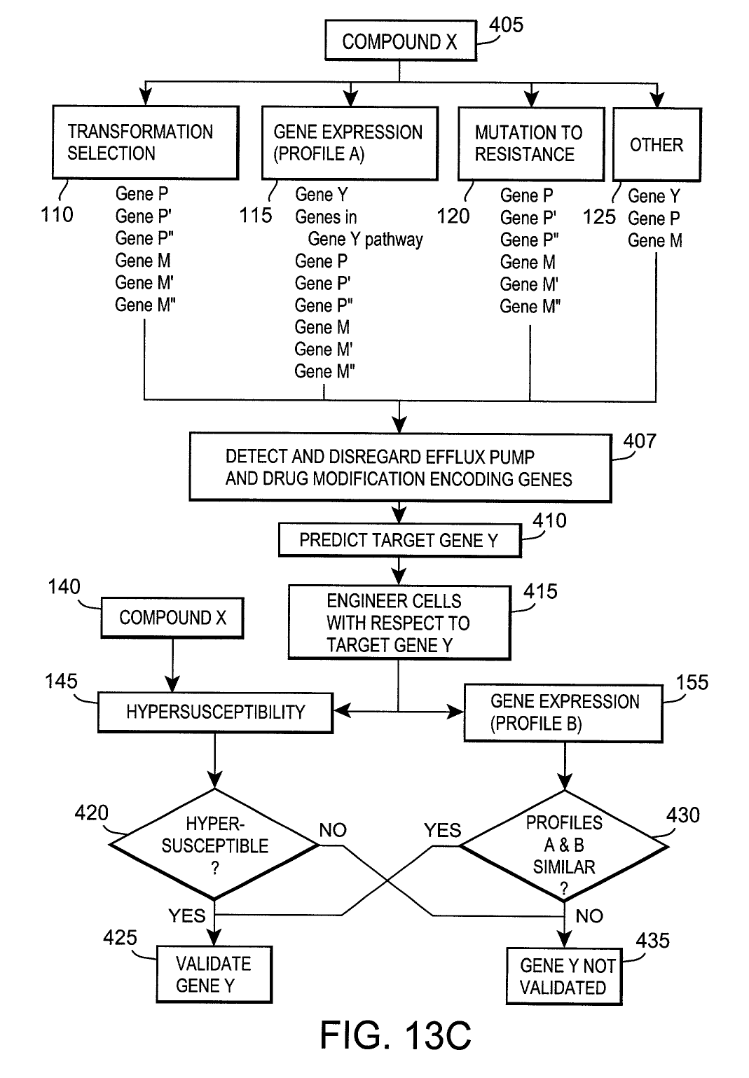

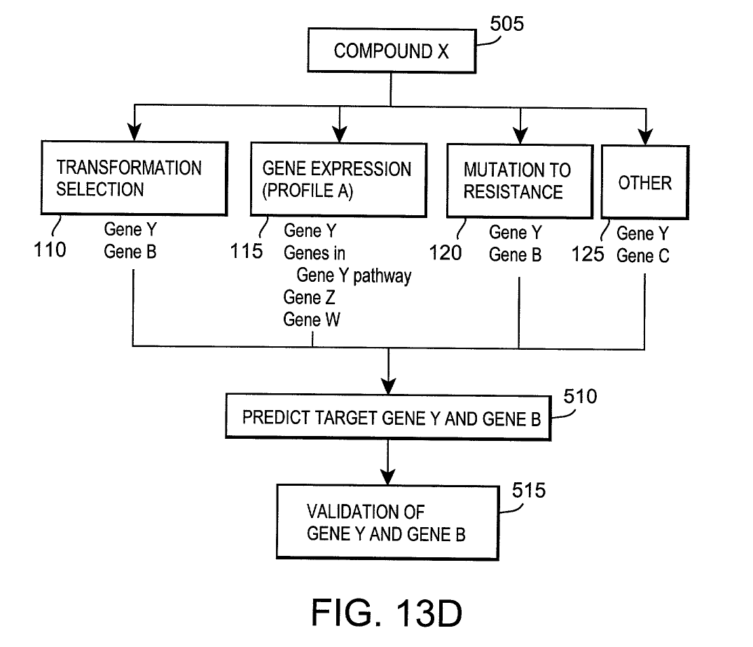

FIG. 13A further illustrates the technique 100 presented in FIG. 12. Initially, cells are exposed to compound X, as shown by step 205. Then, in accordance with the transformation selection process 110, the gene expression process 115, the mutation to resistance process 120, and any other independent target prediction process 125, prospective targets of compound X are identified. In this instance, the transformation selection process 110 identifies two prospective target that are gene products associated with gene Y and gene A. The microarray generated profile associated with the gene expression process 115 identifies a number of potential targets, namely, the gene products associated with gene Z, gene W, gene Y and a number of other genes in the gene Y pathway. The mutation to resistance process 120 also identifies as prospective targets, the gene products associated with gene Y and gene B. The results associated with each of the target gene prediction processes are analyzed and correlated in order to predict one or more predicted targets of compound X, as shown by step 210. In the present instance, a determination is made that the gene product associated with gene Y is the most probable target of compound X, as all of the target prediction processes identified the gene product associated with gene Y.

The gene product associated with gene Y should now be validated. As explained previously, this involves engineering a new population of cells, as shown by step 215. This, in turn, involves deleting or in activating the normal gene Y at locus L₁, and down-regulating an engineered version of gene Y at locus L₂. One or both of the validation processes mentioned above may then be performed using the engineered cells in order to validate the gene product associated with gene Y as a target of compound X. One skilled in the art will understand, however, that executing both validation processes increases the level of confidence associated with the final determination as to whether the gene product is or is not a valid target of compound X.

As stated, the first of the two exemplary validation processes is a hypersusceptibility process, as shown by steps 140 and 145. In executing this procedure, a determination is made, as shown by decision step 220, as to whether the cells become hypersusceptible to compound X as under-expression of engineered gene Y increases. If a determination is made that the cells are, in fact, hypersusceptible, in accordance with the “YES” path out of decision step 220, the gene product associated with gene Y is said to be validated as a target of compound X. If, however, it is determined that the cells are not hypersusceptible,in accordance with the “NO” path out of decision step 220, the gene product associated with gene Y is not validated, as it is less likely that the gene product is a target of compound X.

The second of the two exemplary validation processes is a gene expression process, as shown by step 155, which involves the hybridization of a microarray using labeled cDNA derived from RNA of cells that have been engineered in accordance with step 215. The microarray yields a gene profile (i.e., gene profile B). Gene profile B is then compared with gene profile A, which was generated during step 115. In comparing gene profile A and B, a determination is made, as shown by decision step 230, as to whether the two profiles match, or are substantially similar. If it is determined that the two profiles match, or are substantially similar, in accordance with the “YES” path out of decision step 230, the gene product associated with gene Y is said to be validated as a target of compound X. In contrast, if it is determined that the two profiles do not match, or are not substantially similar, in accordance with the “NO” path out of decision step 230, the gene product associated with gene Y is not validated.

In FIG. 13A, the result is somewhat clear and unambiguous in that each of the various target prediction processes 110-125 identify a single, common gene (i.e., gene Y) or gene product. In practice, however, this does not always occur. FIGS. 13B-13D each represent a variation of the result illustrated in FIG. 13A.

One foreseeable variation is illustrated in FIG. 13B, wherein no rescue of the cells is observed during the transformation selection process 110. Therefore, none of the genes that are over-expressed during the transformation selection process 110, or more particularly, the gene products associated with these gene are identified as prospective targets of compound X. One possible explanation for this is that any over-expressed gene may be a member of a gene pathway, where each gene associated with the pathway must be over-expressed in order to rescue the host cell from compound X.

If, as illustrated in FIG. 13B, no rescue is observed during the transformation selection process 110, the results associated with the other target prediction processes 115-125 should still be analyzed and correlated in accordance with step 310. If, for example, in analyzing the results associated with the other target prediction processes, gene Y and gene B appear to harbor mutations that yield gene products which resist compound X, as identified by the mutation to resistance process 120, while several genes, including gene Y, appear to be up- or down-regulated in the presence of sub-lethal doses of compound X, as identified by the microarray created during the gene expression process 115, then the gene product associated with gene Y is still identified as a prospective target of compound X, as shown by step 310, despite the fact that the transformation selection process 110 failed to identify any prospect gene targets. FIG. 13B further illustrates that the gene product associated with gene Y should be validated in a manner as described above.

A second foreseeable variation of the result illustrated in FIG. 13A is shown in FIG. 13C, wherein efflux pumps or drug modification enzymes are identified as prospective targets of compound X by more than one, if not all of the target gene prediction processes 110-125. In actuality, these gene products are not targets of compound X. Rather, these genes products merely reduce the concentration of compound X. Accordingly, an additional step 407 is employed to determine whether any of the genes or gene products identified by two or more of the target prediction processes 110-125 are or encode efflux or drug modification enzymes. If so, the genes, or more specifically, the correspondence gene products are disregarded as potential targets of compound X.

In the specific example illustrated in FIG. 13C, genes P, P′, P″, M, M′ and M″ are identified by several target prediction processes 110, 115 and 120. In addition, Y is identified by the gene expression process 115 and at least one other target prediction process 125. During step 407, genes P, P′ and P″ are determined to be genes that encode efflux pumps, while genes M, M′ and M″ are determined to be genes that encode drug modification enzymes. Therefore, the gene products (i.e., the efflux pumps and drug modification enzymes) associated with genes P, P′, P″, M, M′ and M″ are disregarded, leaving only the gene product associated with gene Y as a potential target of compound X. Again, the gene product associated with gene Y should be validated as previously described.

Yet another foreseeable variation of the result illustrated in FIG. 13A is shown in FIG. 13D, wherein two or more prospective targets of compound X are identified by the various target prediction processes 110-125. For example, as illustrated in FIG. 13D, both the transformation selection process 110 and the mutation to resistance process 120 identify the gene products associated with gene Y and gene B as prospective targets of compound X. Accordingly, in analyzing and correlating the results of the various target prediction processes 110-125, both the gene products associated with gene Y and gene B are predicted to be targets of compound X, as shown by step 510.

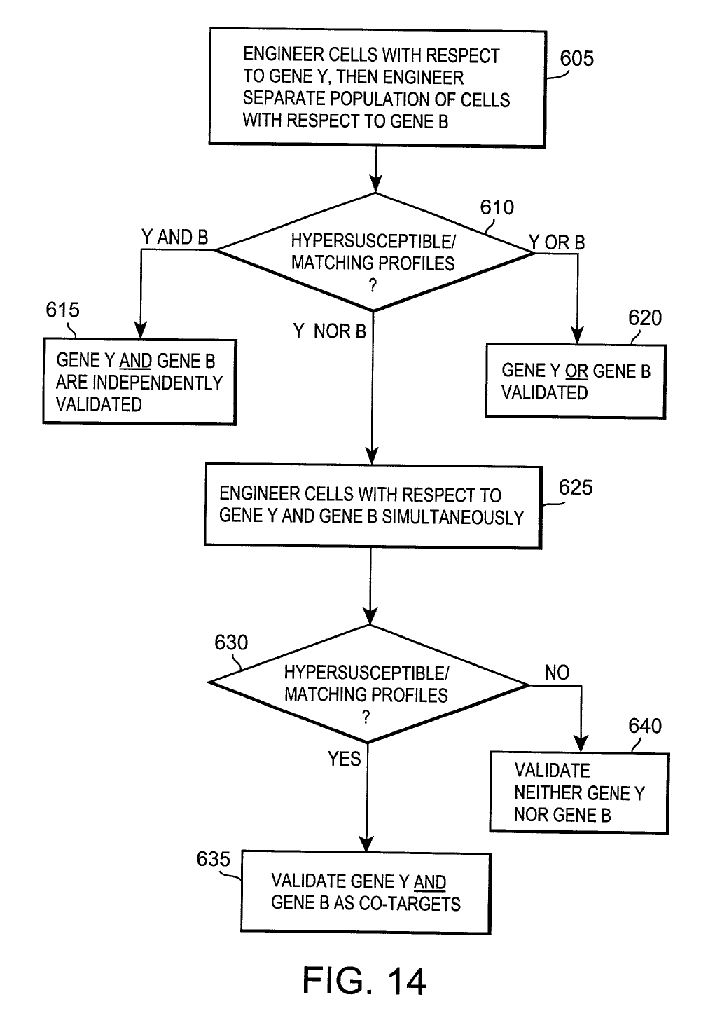

The difference between the technique illustrated in FIG. 13D and the techniques illustrated in FIGS. 12 and FIGS. 13A-13C involves the validation procedure. Because compound X may affect the gene products associated with both gene Y and gene B, engineered cells in which one of these genes is under-expressed, but not the other, may not be hypersusceptible when exposed to compound X or exhibit a gene expression profile that matches a profile associated with normal cells which have been exposed to sub-lethal doses of compound X, the validation processes shown by step 515 must be altered. For example, in validating the gene products of gene Y and gene B, it may be necessary to first validate the gene product of gene Y, then the gene product of gene B, then the gene products of genes Y and B together, as illustrated in FIG. 14.

FIG. 14 illustrates an exemplary technique for validating targets, where, during the prediction phase, the gene products associated with two or more genes have been identified as prospective targets of compound X. As shown by step 605, a first population of cells is engineered with respect to a first one of the two prospective targets (e.g., a gene product associated with gene Y). A second population of cells is then engineered with respect to the second of the two prospective targets (e.g., the gene product associated with gene B). It will be apparent to those skilled in the art that additional populations of cells would be engineered if more than two prospective targets require validation. Then, depending upon whether the hypersusceptibility process, the gene profiling process, or both processes are accomplished to validate the prospective targets, a determination is made, in accordance with decision step 610, as to whether either or both cell populations independently exhibit characteristics of hypersusceptibility when exposed to compound X or result in gene profiles that are similar to the gene profile generated during step 115. If, after separately testing both cell populations, it is determined that both cell populations are hypersusceptible and/or the microarray associated with both cell populations produces a gene profile similar to that which was generated during step 115, in accordance with the “Y AND B” path out of decision step 610, then the gene products associated with both gene Y and gene B are independently validated as targets of compound X. If, instead, it is determined that only one of the two cell populations is hypersusceptible and/or results in a gene profile that is similar to the gene profile generated during step 115, in accordance with the “Y OR B” path out of decision step 610, then only the gene product associated with the gene that was down regulated in that population of cells is validated as a target of compound X.

It is possible, as previously explained, that neither cells engineered with respect to gene Y, nor cells engineered with respect to gene B exhibit hypersusceptibility to compound X or result in a gene profile similar to that which was generated during step 115. The “Y NOR B” path out of decision step 610 represents this scenario. In this instance, additional cells should be engineered with respect to both gene Y and gene B simultaneously, as shown by step 625. These cells are then employed in executing either or both validation processes, that is, the hypersusceptibility process and/or the gene profiling process. Based on the results, a determination is made, in accordance with decision step 630, whether the cells are, in fact, hypersusceptible to compound X or result in a gene profile similar to that which was generated during step 115. If the cells are hypersusceptible when exposed to compound X or the microarray created from these cells produce a gene profile similar to that which was generated during step 115, in accordance with the “YES” path out of decision step 630, then the gene products associated with gene Y and gene B are validated as co-targets of compound X. If the cells are not hypersusceptible when exposed to compound X, or if the microarray created from these cells do not result in a gene profile similar to that which was generated during step 115, in accordance with the “NO” path out of decision step 630, then neither of the gene products associated with gene Y or gene B are validated as targets of compound X.

1.0 Definitions

An “essential gene” is that which when knocked out or made dysfunctional renders the microorganism lacking this essential gene incapable of growth, proliferation or causes the microorganism to die. Examples of such essential genes include, but are not limited to genes which are involved in replication, DNA repair, recombination and transcription, protein synthesis, protein processing and transport, anabolic synthesis of cellular molecules, catabolism of cellular nutrients, synthesis of cell membranes and cell walls, lipid metabolism, protein metabolism, energy metabolism, and cell division.

Said essential genes or products there of are studied and/or identified using the described techniques, which determine target. A “target gene” or a product thereof is predicted and validated using the techniques of the invention. In some instances, more than one target be identified and in some instances, more than one target may be targeted by an inhibitory compound. When more than one gene or product encoded thereby is the target of an inhibitory compound, then each gene is referred to as a “co-target.”

By “gene product” is meant to include mRNAs transcribed from the gene as well as proteins translated from those mRNAs. The preferred molecular target of the invention is a protein encoded by a gene.

By “molecular target” and “drug target” is meant an essential gene, its mRNA or the protein encoded by the gene. Preferred drugs act on a cell by directly interacting with one cellular constituent, however drugs can also act on a plurality of 1, 2, 3, 4, 5, 10, 15, 20 or 50 or more cellular constituents.

By “knock out” is meant a cell wherein a gene is functionally removed from the cell such that the protein encoded thereby can no longer be produced at wild type concentrations or is not functional. The native gene may be, preferably, substantially or entirely removed from the genome of the microorganism.

By “operatively linked” or “operative linkage” is preferably meant when one or more regulatory sequences linked to one or more coding sequences are linked such that the regulatory sequence exercises control on the coding sequence without effecting the activity of other coding sequences or other operons. Less preferred would be if the regulatory sequence linked to the coding sequence does impact the activity of other coding sequences or other operons. Less preferred would be recombinant constructs which exist as freely replicating extrachromosomal elements. Preferred constructs are those which are inserted into the chromosome of the organism.

By “drugs” is meant any compound or chemical of any degree of complexity that perturbs or modulates a microorganism, whether by known or unknown mechanisms and whether or not the drug can be utilized therapeutically. Potential drugs can be screened in the form of drug libraries. Drugs include typical small molecules, naturally occurring factors (e.g., endocrine, paracrine, or autocrine factors), intracellular factors, and compounds isolated from natural sources (e.g., Actinomycetes, fungi, herbs or herbal extracts). These drugs can be activating drugs, which increase or stimulate the activities of a protein as well as inhibiting drugs, which decrease or down-regulate the activity of protein. Preferred drugs are those which inhibit or prevent the growth or proliferation of a microorganism or kill the microorganism.

The terms “antibacterial,” “antimicrobial” and “antibiotic” are meant to include chemicals and compounds which inhibit and/or stop bacterial and/or microbial growth and/or proliferation. This includes bactericidal and bacteriostatic agents. By “bactericidal” is meant the ability to kill the bacteria. By “bacteriostatic” is meant the ability to prevent or significantly retard growth of bacteria.

By “hypersensitive,” “hypersensitivity,” “hypersusceptible” and “hypersusceptibility” is meant a host cell in which a gene is being under-expressed and, compared to cells with normal levels of gene expression, is more sensitive to inhibition of growth or viability with respect to agents which affect the activity of the gene product.

By “under-expressed” or “under-expression” is meant the decreased expression of a gene at levels which range below the wild-type levels observed for that gene in that particular organism.

By “resistant” or “resistance” is meant the acquired ability to survive and/or proliferate by a cell despite the presence of a compound to which the cell is typically sensitive.

By “over-expressed” is meant the increased expression of a gene at levels which range above the wild-type concentrations observed for that gene in that organism or cell. Preferably, said over-expressed gene increases the resistance of the organism to a compound or composition that otherwise inhibits the activity of the gene product.

By “vector” is meant a DNA molecule that can be replicated in a cell and that can serve as the vehicle for transfer to such a cell of DNA that has been inserted into it by recombinant techniques. The vectors of the instant invention will contain at least a resistance cassette. The vector will preferably contain a wild-type copy of the gene encoding the putative molecular target as well as suitable promoters and operators operably linked together with the gene. The vector will potentially further contain antibiotic resistence genes and methods of expressing those genes.

By “regulated promoter” is meant a promoter to which RNA polymerase binds that is induced by an agent such as an inducer, or repressed by an agent such as a repressor, or induced or repressed by a condition such as heat. Such induction or repression causes the gene operably linked to said promoter to be more or less transcribed and then translated. One example of a regulator used, for example in bacteria, is arabinose.

By “cassette” is meant a genetic structure into which other genetic units can be inserted or removed. These can be in the form of “resistance cassettes” or “hypersensitivity cassettes.” These cassettes will comprise a 5′ and 3′ loci, an inducible promoter, either a gene or a multiple cloning site and a gene conferring antibiotic resistance. When the essential gene is put in combination with a regulatable promoter such that it is over-expressed, a resistance cassette is constructed. When the essential gene is put in combination with a regulatable promoter such that it is under-expressed, a hypersensitive cassette is constructed.

By “pathogen” or “organism” or “microorganism” is meant to include bacteria, as well as fungi, amebas, protozoa and other infectious agents listed herein. By “protozoa” is meant to include rhizopods, flagellates and ciliates. “Fungi” includes all members of myxomycetes, phycomycetes, ascomycetes and basidiomycetes. A pathogen includes any organism capable of infecting and damaging a mammalian host, and, in particular, includes gram-negative and gram-positive bacteria. Thus, the term includes both virulent pathogens which, for example, which can cause disease in a previously healthy host and opportunistic pathogens, which can only cause disease in a weakened or otherwise compromised host. By “cell” is meant to include pathogens, organisms and microorganisms listed herein as well as cells from multicellular eukaryotes such as mammals, agricultural animals, primates, humans, malignant cells from any animal (e.g., murine, equine, porcine, caprine, bovine, canine, feline, primate, ovine, rodent, etc.)

“Host cell” is a cell, preferably a microorganism, which has been transformed or transfected, or is capable of transformation or transfection by an exogenous polynucleotide sequence.

By “strain” is meant an isolate of a pathogen which resembles the pathogen in the major properties that define the type, but differs in minor properties such as vector species specificity, symptoms regulated, serological and genetic properties.

The term “bacterial infection” refers to the invasion of the host animal by pathogenic bacteria. This includes the excessive growth of bacteria which are normally present in or on the body of an animal. More generally, a bacterial infection can be any situation in which the presence of a bacterial population(s) is damaging to a host animal. Thus, an animal is “suffering” from a bacterial infection when excessive numbers of bacteria are present in or on an animal's body, or when the effects of the presence of a bacterial population(s) is damaging the cells or other tissue of an animal.

“Disease(s)” in one sense is meant to include human conditions caused by or related to infection by a bacteria, fungi, or other pathogen listed herein. However, pathogen regulated diseases and conditions are also meant to include infections of other animals, such as mammals (e.g., domesticated animals such as cattle, horses, sheep, goats, pigs, rabbits, murine species, as well as other primates), reptiles, birds and amphibians.

The terms “therapeutically effective amount” or “pharmaceutically effective amount” mean the amount of a drug or pharmaceutical compound that will elicit the biological or medical response of a tissue, system or animal being treated with said drug or compound that is being sought by the researcher clinician. Preferably, the effective amount will inhibit or prevent infection by the pathogens of the instant invention.

“Treating,” in this context, refers to administering a pharmaceutical composition for prophylactic and/or therapeutic purposes. The term “prophylactic treatment” refers to treating a patient who is not yet infected, but who is susceptible to, or otherwise at risk, of a particular infection. The term “therapeutic treatment” refers to administering treatment to a patient already suffering from a pathogen-regulated infection.

The term “administration” or “administering” refers to a method of giving a dosage of a pharmaceutical composition to an animal to treat a pathogen regulated infection or prevent said infection, where the method is, e.g., topical, oral, intravenous, transdermal, intraperitoneal, or intramuscular. The preferred method of administration can vary depending on various factors, the components of the pharmaceutical composition, the site of the potential or actual infection, the pathogen involved, and the severity of the actual infection.

The term “biochemical pathway” refers to a connected series of biochemical reactions normally occurring in a host cell, or more broadly, a cellular event such as cellular division or DNA replication. Typically, the steps in such a biochemical pathway act in a coordinated fashion to produce a specific product or products or to produce some other particular biochemical action. Such a biochemical pathway requires the expression product of a gene (e.g., essential gene) if the absence of that expression product either directly or indirectly prevents the completion of one or more steps in that pathway, thereby preventing or significantly reducing the production of one or more normal products or effects of that pathway. Thus, an agent specifically inhibits such a biochemical pathway requiring the expression product of a particular gene, if the presence of the agent stops or substantially reduces the completion of the series of steps in that pathway. Such an agent, may, but does not necessarily, act directly on the expression product of that particular gene.

The term “antibacterial agent” refers to both naturally occurring antibiotics produced by microorganisms to suppress the growth of other microorganisms, and agents synthesized or modified in the laboratory which have either bactericidal or bacteriostatic activity, e.g., β-lactam antibacterial agents, glycopeptides, macrolides, quinolones, tetracyclines, and aminoglycosides. In general, if an antibacterial agent is “bacteriostatic,” it means that the agent essentially stops bacterial cell growth (but does not kill the bacteria); if the agent is “bacteriocidal,” it means that the agent kills the bacterial cells (and may stop growth before killing the bacteria). By the terms “modulate,” “regulate” or “perturb” is meant a change or an alteration in the biological activity of an essential gene or the protein encoded thereby. Modulation and regulation may be an increase or a decrease in said activity.

By “perturb” or “modulate” is meant the ability of a compound to alter the activity of a molecular target away from what the wild-type activity for said target. For examples, modulation can occur by impacting the activity of a protein (e.g., down-regulating its ability to act on other proteins, bind to its cognate ligand, etc.), altering transcription of a gene encoding the target, and altering translation of the mRNA of the gene encoding the target. Modulation is preferably inhibitory to a cell's activity (e.g., bacteriostatic or bacteriocidal).

By “agonist” is meant a molecule or compound which increases or activates the amount of, or prolongs the duration of the activity of the protein encoded by the molecular target of a pathogen. Agonists may include proteins, nucleic acids, carbohydrates, immunoglobulins or any other molecules which up-regulate the activity of the molecular target.

By “antagonist” is meant a molecule or compound which decreases or inhibits the amount of, or shortens the duration of the activity of the protein encoded by the molecular target of a pathogen. Antagonists may include proteins, nucleic acids, carbohydrates, immunoglobulins or any other molecules which down-regulate the activity of the molecular target.

A “carrier” or “excipient” is a compound or material used to facilitate administration of the compound, for example, to increase the solubility of the compound. Solid carriers include, e.g., starch, lactose, dicalcium phosphate, sucrose, and kaolin. Liquid carriers include, e.g., sterile water, saline, buffers, non-ionic surfactants, and edible oils such as peanut and sesame oils. In addition, various adjuvants, such as are commonly used in the art, may be included. These and other such compounds are described in the literature, e.g., in the MERCK INDEX, Merck & Company, Rahway, N.J. Considerations for the inclusion of various components in pharmaceutical compositions are described, e.g., in GOODMAN AND GILMAN'S: THE PHARMACOLOGICAL BASIS OF THERAPEUTICS, 9th Ed., Pergamon Press (1995).

Use of the term “isolated” indicates that a naturally occurring material or organism (e.g., a DNA sequence or a protein) has been removed from its normal environment. Thus, an isolated DNA sequence has been removed from its usual cellular environment, and may, for example, be in a cell-free solution or placed in a different cellular environment. For a molecule, such as a DNA sequence, the term does not imply that the molecule (sequence) is the only molecule of that type present.

The terms “isolated” and “purified” as applied to proteins herein refers to a composition wherein the desired protein or nucleic acid comprises at least 35% of the total protein or nucleic acid component in the composition. The desired protein or nucleic acid preferably comprises at least 40%, more preferably at least about 50%, more preferably at least about 60%, still more preferably at least about 70%, even more preferably at least about 80%, even more preferably at least about 90%, and most preferably at least about 95% of the total protein or nucleic acid component. The composition may contain other compounds such as carbohydrates, salts, lipids, solvents, and the like, without affecting the determination of percentage purity as used herein.

It is also advantageous for some purposes that an organism or molecule (e.g., a nucleotide sequence) be in purified form. The term “purified” does not require absolute purity; instead, it indicates that the sequence, organism, or molecule is relatively purer than in the natural environment. Preferably 80% purity is desired however 90% purity is preferred, and even more preferably 95% purity. Thus, the claimed DNA could not be obtained directly from total cell DNA or from total cell RNA. The claimed DNA sequences are not naturally occurring, but rather are obtained via manipulation of a partially purified naturally occurring substance (genomic DNA clones). The construction of a genomic library from chromosomal DNA involves the creation of vectors with genomic DNA inserts and pure individual clones carrying such vectors can be isolated from the library by clonal selection of the cells carrying the library.

In a further aspect, this invention provides an isolated or purified DNA sequence which is the same as or complementary to a gene homologous to any gene of a pathogen or cell, where the function of the expression product of the homologous gene is the same as the function of the product of one of the above-identified genes. In general, such a homologous gene will have a high level of nucleotide sequence similarity and, in addition, a protein product of homologous gene will have a significant level of amino acid sequence similarity. Preferably, the gene is identical to the wild-type form of the gene which has been knocked out of the host-cell prior or subsequent to insertion of the gene, which is under the regulation of an inducible promoter.

2.0 Microorganisms and Genes

The preferred cells of the invention to determine the molecular targets of compounds which target preferably essential genes or the products encoded thereby. The molecular targets will preferably be those of microorganisms, however, this assay can also be utilized to study viruses, protozoa, and more complex eukaryotic cells such as human cells and tumor cells.

Of the preferred microorganisms, they can be prokaryotic or eukaryotic. Preferred prokaryotic organisms can be either Gram-positive or Gram-negative. These organisms can include, but are not limited to: Staphylococcus, Streptococcus, Enterococcus, Neisseria, Branhamella, Listeria, Bacillus, Corynbacterium, Erysipelothrix, Gardnerella, Mycobacterium, Nocardia, Enterobacteriaceae, Escherichia, Salmonella, Shigella, Yersinia, Enterobacter, Klebsiella, Citrobacter, Serratia, Providencia, Proteus, Morganella, Edwardsiella, Erwinia, Vibrio, Aeromonas, Helicobacter, Campylobacter, Eikenella, Pasteurella, Pseudomonas, Burkholeria, Stenotrophomonas, Acinetobacter, Ralstonia, Alcaligenes, Moraxella, Legionella, Francisella, Brucella, Haemophilus, Bordetella, Clostridium, Bacteroides, Porphyromonas, Prevotella, Fusobacterium, Borrelia, Chlamydia, Rickettsia, Ehrlichia, and Bartonella.

Of course, the preferred members of these genuses would be those that are most responsible for causing diseases in their hosts such as humans, agricultural animals, domesticated animals, and the like. Such disease causing members of these bacterial genuses include, but are not limited to, Bacillus subtilis, Escherichia coli, Enterobacter cloacae, Haemophilus influenzae, Klebsiella pneumoniae, Klebsiella oxytoca, Proteus mirabilis, Proteus vulgaris, Morganella morganii, Helicobacter pylori, Pseudomonas aeruginosa, Staphylococcus aureus, Staphylococcus epidermidis, Streptococcus pneumoniae, Streptococcus pyogenes, and Streptococcus agalactiae.

By “bacteria” is meant to include “gram-positive” and “gram-negative” bacteria. By “gram positive bacteria” is meant to include those bacteria which resist decolorization with a polar, organic solvent and remain stained by the Gram stain. “Gram-negative bacteria” rapidly decolorize after exposure to the solvent. “Bacteria” also is meant to include all members of “eubacteria” and “archaebacteria.” Members of “eubacteria” contemplated by the invention include: aquifecales, thermotogales, thermodesulfobacterium, thermus-deinococcus group, chloroflecales, cyanobacteria, firmicutes, leptospirillum group, synergists, chlorobium-flavobacteria group, verrucomicrobia, chlamydia, planctomycetales, flexistipes, fibrobacter group, spirochetes, and proteobacteria (e.g., α-, β- δ-, ε- and γ-proteobacteria). The “archaebacteria” contemplated by the invention include: methanogens, halophiles, and thermophiles. Additional bacteria are presented in Table 1 below. They are classified by Order, Suborder, Family and Genus.

TABLE 1

| PHOTOTROPHIC BACTERIA | GLIDING BACTERIA | SHEATHED BACTERIA |
|---|---|---|
| Rhodospirllales (Order) | Myxobacterales (Order) | Sphaerotilus (Genus) |
| Rhodospirillineae (Suborder) | Myxococcaceas (Family) | Lepotothrix |
| Rhodospirillaceae (Family) | Myxococcus (Genus) | Streptothrix |
| Rhodospirillum (Genus) | Archangiaceae (Family) | Lieskeela |
| Rhodopseudomonas | Archangium (Genus) | Phragmidiothrix |
| Rhodomicrobium | Cystobacteraceau (Family) | Crenothrix |
| Chromatiaceae (Family) | Cystobacter (Genus) | Clonothrix |
| Chromatium (Genus) | Melittangium | |
| Thiocystis | Stigmatella | |
| Thiosarcina | Polyangiaceae (Family) | |
| Thiospirillum | Polyangium (Genus) | |
| Thiocapsa | Nannocystis | |
| Lamprocystis | Chondromyces | |
| Thiodictyon | Cytophagales (Order) | |
| Thiopedia | Cytophagaceau (Family) | |
| Amoebobacter | Cytophaga (Genus) | |
| Ectothiorhodospira | Flexibacter | |
| Chlorobiineae (Suborder) | Herpetosiphon | |
| Chlorobiaceae (Family) | Flexithrix | |
| Chlorobium | Saprospira | |
| Prosthecochloris | Sporocytophaga | |
| Chloropseudomonas | Beggiatoaceae (Family) | |
| Pelodictyon | Beggiatoa (Genus) | |
| Clathrochloris | Vitreoscilla | |
| Chlorochromatium (Addenda) | Thioploca | |
| Cylindrogloea (Addenda) | Simonsiellaceae (Family) | |
| Chlorobacterium (Addenda) | Simonsiella (Genus) | |
| | Alysiella | |
| | Leucotrichaceae (Family) | |
| | Leucothrix (Genus) | |
| | Thiothrix | |
| | Incertae sedis | |
| | Toxothirix (Genus) | |
| | Familiae incertae sedis | |
| | Achromatiaceae | |
| | Achromatium (Genus) | |
| | Pelonemataceae | |
| | Pelonema (Genus) | |
| | Achroonema | |
| | Peloploca | |
| | Desmanthos | |
| BUDDING AND/OR | | SPIRAL AND CURVED |
| APPENDAGES BACTERIA | SPIROCHETES | BACTERIA |
| Hyphomicrobium (Genus) | Spirochaetales (Order) | Spirillaceae (Family) |
| Hyphomonas | Spirochaetaceae (Family) | Spirillum (Genus) |
| Pedomicrobium | Spirochaeta (Genus) | Campylobacter |
| Caulobacter | Cristispira | Incertae sedis |
| Asticcacaulis | Treponema | Bdellovirbio (Genus) |
| Ancalomicrobium | Borrelia | Microcyclus |
| Prosthecomicrobium | Leptospira | Pelosigma |
| Thiodendron | | Brachyarcus |
| Pasteuria | | |
| Blastobacter | | |
| Seliberia | | |
| Gallionella | | |
| Nevskia | | |
| Planctomyces | | |
| Metallogenium | | |
| Caulococcus | | |
| Kusnezonia | | |
| | GRAM-NEGATIVE | |
| GRAM-NEGATIVE AEROBIC | FACULTATIVELY ANAEROBIC | GRAM-NEGATIVE ANAEROBIC |
| RODS AND COCCI | RODS | BACTERIA |
| Pseudomonadaceau (Family) | Enterobacteriacease | Bacteroidaceae (Family) |
| | (Family) | |
| Pseudomonas (Genus) | Escherichia (Genus) | Bacteriodes (Genus) |
| Xanthomonas | Edwardsiella | Fusobacterium |
| Zoogloea | Citrobacter | Leptotrichia |
| Gluconobacter | Salmonella | Incertae sedis |
| Azotobacteraceae (Family) | Shigella | Desulfovibrio (Genus) |
| Azotobacter (Genus) | Klebsiella | Butyrivibrio |
| | Enterobacter | Succinivibrio |
| Azomonas | Hafnia | Succinimonas |
| Beijernickia | Serratia | Lachnospira |
| Derxia | Proteus | Selenomonas |
| Rhizobiaceae (Family) | Yersinia | |
| Rhizobium (Genus) | Erwinia | |
| Agrobacterium | Vibrionaceae (Family) | |
| Methylomonadaceae (Family) | Vibrio (Genus) | |
| Methylomonas (Genus) | Aeromonas | |
| Methylococcus | Plesiomonas | |
| Halobacteriaceae (Family) | Photobacterium | |
| Halobacterium (Genus) | Lucibacterium | |
| Halococcus | Incertae sedis | |
| Incertae sedis | Zymomonas (Genus) | |
| Alcaligenes (Genus) | Chromobacterium | |
| Acetobacter | Flavobacterium | |
| Brucella | Haemophilus | |
| Bordetella | Pasteurella | |
| Francisella | Actinobacillus | |
| Thermus | Cardiobacterium | |
| | Streptobacillus | |
| | Calymmatobacterium | |
| | GRAM NEGATIVE | |
| GRAM-NEGATIVE COCCI AND | CHEMOLITHOTROPHIC | METHANE-PRODUCING |
| COCCOBACILLI | BACTERIA | BACTERIA |
| Neisseriaceae (Family) | Nitrobacteraceae (Family) | Methanobacteriaceae |
| | | (Family) |
| Neisseria (Genus) | Nitrobacter (Genus) | Methanobacterium (Genus) |
| Branhamella | Nitrospina | Methanosarcina |
| Moraxella | Nitrococcus | Methanococcus |
| Acinetobacter | Nitrosomonas | |
| Incertae sedis | Nitrospira | |
| Paracoccus (Genus) | Nitrosococcus | |
| Lampropdeia | Nitrosolobus | |
| | Organisms Metabolizing Sulfur | |
| | Thiobacillus (Genus) | |
| | Sulfolobus | |
| | Thiobacterium | |
| | Macromonas | |
| | Thiovulum | |
| | Thiospira | |
| | Siderocapsaceae (Family) | |
| | Siderocapsa | |
| | Naumanniella | |
| | Ochrobium | |
| | Siderococcus | |
| | | GRAM-POSITIVE, |
| | ENDOSPORE-FORMING RODS | ASPOROGENOUS ROD- |
| GRAM-POSITIVE COCCI | AND COCCI | SHAPED BACTERIA |
| Micrococcaceae (Family) | Bacillacea (Family) | Lactobacillaceae (Family) |
| Micrococcus (Genus) | Bacillus (Genus) | Lactobacillus (Genus) |
| | Sporolactobacillus | Incertae sedis |
| | Clostridum | Listeria (Genus) |
| | Desulfotomaculum | Erysipelothrix |
| | Sporosarcina | Caryophanon |
| | Intertae sedis | |
| | Oscillospira (Genus) | |
| ACTINOMYCETES AND | | |
| RELATED | | |
| ORGANISMS | RICKETTSIAS | MYCOPLASMAS |
| Corvneform Group | Rickettsiales (Order) | Mollicutes (Class) |
| of Bacteria | | |
| Corynebacterium | Rickettsiaceae (Family) | Mycoplasmatales (Order) |
| Arthrobacter | Rickettsieae (Tribe) | Mycoplasmataceae (Family) |
| Incertae sedis | Rickettsia (Genus) | Mycoplasma (Genus) |
| Brevi bacterium | Rochalimaea | Acholeplasmataceae |
| Micro bacterium | Coxiella | (Family) |
| Cellulomonas | Ehrlichieae (Tribe) | Acholeplasma (Genus) |
| Kurthia | Ehrlichia (Genus) | Incertae sedis |
| Propionibacteriaceae (Family) | Cowdria | Thermoplasma (Genus) |
| Propionibacterium | Neorickettsia | Incertae sedis |
| (Genus) | Wolbachieae (Tribe) | Spiroplasma (Genus) |
| Eubacterium | Wolbachia (Genus) | |
| Actinomycetales (Order) | Symbiotes | |
| Actinomycetaceae (Family) | Blattabacterium | |
| Actinomyces (Genus) | Rickettsiella | |
| Arachnia | Bartonellaceae (Family) | |
| Bifidobacterium | Bartonella (Genus) | |
| Bacterionema | Grahamella | |
| Rothia | Anaplasmataceae (Family) | |
| Mycobacteriaceae (Family) | Anaplasma (Genus) | |
| Mycobacterium (Genus) | Paranaplasma | |
| Frankiaceae (Family) | Aegyptionella | |
| Frankia (Genus) | Haemobartonella | |
| Actinoplanaceae (Family) | Eperythrozoon | |
| Actinoplanes (Genus) | Chlamydiales (Order) | |
| Spirillospora | Chlamydiaceae (Family) | |
| Streptosporangium | Chlamydia (Genus) | |
| Amorphosporangium | | |
| Ampullariella | | |
| Pilimelia | | |
| Planomonospora | | |
| Planobispora | | |
| Dactylosporangium | | |
| Kitastoa | | |
| Dermatophilaceae (Family) | | |
| Dermatophilus (Genus) | | |
| Geodermatophilus | | |
| Nocardiaceae (Family) | | |
| Nocardia (Genus) | | |
| Pseudonocardia | | |
| Streptomycetaceae (Family) | | |
| Streptomyces (Genus) | | |
| Streptoverticillium | | |
| Sporichthya | | |
| Microellobosporia | | |
| Micromonosporaceae | | |
| (Family) | | |
| Micromonospora | | |
| (Genus) | | |
| Thermoactinomyces | | |
| Actinobifida | | |
| Thermomonospora | | |
| Microbispora | | |
| Micropolyspora | | |

The categorization of the individual orders, suborders, families and genuses may change over time as more information is learned regarding their phylogenic relationships.

The essential genes of the invention are those which are discussed above. They can encode proteins, such as, topoisomerases, nucleases, recombinases, primases, helicases, DNA and RNA polymerases, histone modifying enzymes, kinases, phosphatases, phosphorylases, acetylases, deacetylases, formylases, deformylases, chaperonins, ion transporters, cytoskeletal elements, colicins, cytochromes, ribosomal proteins, transfer RNAs (tRNA), ribosomal RNAs (rRNA), proteases, epimerases, rotamases, synthases, racemases, dehydrogenases, transferases, ligases reductases, oxidases, transglycocylases, transpeptidases, peptidases, GTPases, ATPases, translocases, ribonucleases, transcription factors, sigma factors, ribosomal release factors and proteins involved in the structure of the protein. Examples of specific proteins encoded by essential genes and their host organism include but are not limited to those presented in Table 2 below:

TABLE 2
Identified Essential Genes and Associated Drugs

| Gene | Organism | Function | Drug | Reference |
|---|---|---|---|---|
| alrA | Mycobacterium | D-alanine | D-4-amino- | Cáceres et al., J. |
| | | racemase | isoxazolidone | Bacteriol. 179: 5046-55 |
| | | | | (1997) |
| ATCC | Aspergillus | AN80, | | WO 99/24580 to Gavrias |
| No. | nidulans | ribosomal | | et al. |
| 209472 | | | | |
| ATCC | Aspergillus | AN17, | | WO 99/24580 to Gavrias |
| No. | nidulans | ribosomal | | et al. |
| 209484 | | | | |
| ATCC | Aspergillus | AN97, | | WO 99/24580 to Gavrias |
| No. | nidulans | ribosomal | | et al. |
| 209471 | | | | |
| ATCC | Aspergillus | AN85, | | WO 99/24580 to Gavrias |
| No. | nidulans | ribosomal | | et al. |
| 209473 | | | | |
| folA | E. coli | dihydrofolate | Trimethoprim | Neuwald et al., Gene 125: |
| | | reducatase | (Tp), | 69-73 (1993) |
| | | (DHFR) | methotrexate | |
| | | | (MTX) | |
| gpt | S. pombe | UDP-N- | tunicamycin | Scocca et al., |
| | | acetylglucos- | (TM) | Glycobiology 7: 1181-9 |
| | | aminyl: | | (1997) |
| | | dolichyl- | | |
| | | phosphate N- | | |
| | | acetylglucos- | | |
| | | aminyl | | |
| | | phosphoryl | | |
| | | transferase (L- | | |
| | | G1PT) | | |
| grpE+ | bacteriophage | heat shock | | Ang et al., J. Bacteriol. |
| | lambda and E. | protein | | 171: 2748-55 (1989) |
| | coli | | | |
| gyrA | Streptococcus | GyrA subunit | gatifloxacin, | Fukuda et al., |
| | pneumoniae | of DNA | sparfloxacin | Antimicrobial Agents & |
| | | gyrase | | Chemotherap. 43: 410-12 |
| | | | | (1999) |
| gyrB | Streptococcus | DNA gyrase | fluoroquinolone | Jorgensen et al., |
| | | | (FQ) | Antimicrobiol. Agents & |
| | | | | Chemo. 43: 329-34 |
| | | | | (1999) |
| hbal | S. pombe | similar to S. | brefeldin A | Turi et al., J. Biol. Chem. |
| | | cerevisiae | | 271: 9166-71 (1996) |
| | | YBB1 | | |
| IPC1 | S. cerevisiae, | inositolphos- | Aureobasidin A^{R} | Heidler et al., Biochimica |
| | Candida and | phorylceramide | | et Biophvsica Acta 1500: |
| | Cryptococcus | synthase | | 147-52 (2000) |
| mtrAB | Streptomyces | | mithramycin | Fernandez et al., Mol. |
| | argillaceus | | | Gen. Genetics 251: 692-8 |
| | | | | (1996) |
| murA | | UDP-N- | fosfomycin | Horii et al., Antimicrobial |
| | | acetylgluco- | | Agents & Chemo. |
| | | samine | | 43: 789-93 (1999) |
| | | enolpyruvoyl | | |
| | | transferase | | |
| nadD | Salmonella | NAD/NADP | 6-aminonicotin- | Hughes et al., J. |
| | typhimurium | biosynthesis | amide | Bacteriol. 154: 1126-36 |
| | | | | (1983) |
| nalA | E. coli | | nalidixic acid | Kreuzer et al., Mol. & |
| | | | | Gen. Genetics 167: 129- |
| | | | | 37 (1978) |
| parC | Streptococcus | ParC subunit | trovafloxacin, | Fukuda et al., |
| | pneumoniae | of TopoIV | levoflocacin, | Antimicrobial Agents and |
| | | | norfloxacin, | Chemo. 43: 410-2 (1999) |
| | | | ciprofloxacin | |
| parE | S. pneumoniae | topoisomerase | fluoroquinolone | Jorgensen et al., |
| | | IV | (FQ) | Antimicrobiol. Agents & |
| | | | | Chemo. 43: 329-34 |
| | | | | (1999) |
| psul | S. pombe | | | Omi et al., Biochem. |
| | | | | Biophys. Res. Comm. |
| | | | | 262: 368-74 (1999) |
| RPB7 | Saccharomyces | Rpb7 subunit | bleomycin | He et al., Biochem. & |
| | cerevisiae | of RNA | | Cell Biol. 77: 375-82 |
| | | polymerase II | | (1999) |
| | | complex | | |
| rplD | E. coli, Y. | L4 ribosomal | | Chittum and Champney, |
| | pseudotuber- | protein | | J. Bacteriol. 176: 6192-8 |
| | culosis,M. | | | (1994) |
| | capricolum | | | |
| rplV | E. coli, M. | L22 ribosomal | | Chittum and Champney, |
| | capricolum, B. | protein | | J. Bacteriol. 176: 6192-8 |
| | stearother- | | | (1994) |
| | mophilus, Pea | | | |
| | (chloro.)^{c}, | | | |
| | liverwort | | | |
| | (chloro.), C. | | | |
| | paradoxa, red | | | |
| | alga (chloro.), | | | |
| | spinach | | | |
| | (chloro.), | | | |
| | tobacco | | | |
| | (chloro.), rice | | | |
| | (chloro.), rat | | | |
| | (liver, | | | |
| | Halobacterium | | | |
| | m. | | | |
| rpoB | E. coli | RNA | rifampicin (Rif^{R}), | Severinov et al., J. Biol. |
| | | polymerase β | streptolydigin | Chem. 268: 14820-5 |
| | | subunit | (St1^{R}) | (1993) |
| secA | E. coli | protein | sodium azide (azi | Oliver et al., Proc. Natl. |
| | | transport | mutant) | Acad. Sci. USA 87: |
| | | across E. coli | | 8227-31 (1990) |
| | | plasma | | |
| | | membrane | | |
| TUB2 | S. cerevisiae | structural gene | benomyl (Ben^{R}) | Thomas et al., Genetics |
| | | encoding β- | | 111: 715-34 (1985) |
| | | tubulin | | |
| uppS | multiple | undecaprenyl | | Apfel et al., J. Bacteriol. |
| | microorgan- | pyrophosphate | | 181: 483-92 (1999) |
| | isms | synthetase | | |
| VAN2 | S. cerevisiae | | orthovanadate | Kanik-Ennulat et al., |
| | | | | Genetics 140: 933-43 |
| | | | | (1995) |
| ycfB | gram negative/ | | | Arigoni et al., Nature |
| | positive | | | Biotech. 16: 851-6 |
| | bacteria | | | (1998); WO 99/54462 to |
| | | | | Arigoni et al. |
| ydiE | B. subtilis | | | Arigoni et al., Nature |
| | | | | Biotech. 16: 851-6 (1998) |
| YEF-3 | S. cerevisiae | elongation | | Qin et al., J. Biol. Chem. |
| | | factor for | | 265: 1903-12 (1990) |
| | | protein | | |
| | | synthesis | | |
| yfil | gram negative/ | | | Arigoni et al., Nature |
| | positive | | | Biotech. 16: 851-6 (1998) |
| | bacteria | | | |
| ygjD | gram negative | sialoglyco- | | WO 99/54470 to Arigoni |
| | and gram | protease | | et al. |
| | positive | | | |
| | bacteria | | | |
| YGR251w | S. cerevisiae | | | Sartori et al., Yeast 16: |
| | | | | 255-65 (2000) |
| yhbZ | gram negative/ | | | Arigoni et al., Nature |
| | positive | | | Biotech. 16: 851-6 (1998) |
| | bacteria | | | |
| yihA | gram negative/ | | | Ariaoni et al., Nature |
| | positive | | | Biotech. 16: 851-6 (1998) |
| | bacteria | | | |
| yjeQ | gram negative | | | WO 99/54473 to Arigoni |
| | and gram | | | et al. |
| | positive | | | |
| | bacteria | | | |
| YLL031c | S. cerevisiae | likely amino | | Zhang et al., Yeast 15: |
| | | peptidase | | 1287-96 (1999) |
| yneS | gram negative | S-yneS | | WO 2000/20627 to Fritz |
| | bacteria | polypeptide | | et al. |
| YOR319w | S. cerevisiae | part of | | Pearson et al., Yeast 14: |
| | | spliceosome | | 391-9 (1998) |
| | | complex | | |
| YPRO41 | S. cerevisiae | protein | | Waskiewicz- |
| w (TIF5) | | synthesis | | Staniorowska et al., Yeast |
| | | initiation | | 14: 1027-39 (1998) |
| | | translation | | |
| | | factor | | |

Other essential genes would be those especially that are members of a hierarchical biological pathway. Hierarchical pathways have no feedback loops, but are instead are pathways in which its cellular constituents can be arranged into a hierarchy of numbered levels so that cellular constituents belonging to a particular numbered level can be influenced only by cellular constituents belonging to levels of lower numbers. A hierarchical pathway originates from the lowest numbered cellular constituents. In contrast, a non-hierarchical pathway has one or more feedback loops. A feedback loop is a subset of cellular constituents of the pathway, wherein each constituent of the feedback loop influences and also is influenced by other constituents of the feedback loop. Accordingly, genes which are involved in non-hierarchical pathways with multiple feedback loops are unlikely to be preferred genes as their presence may not be essential to the growth and proliferation of the microorganism.

Determining whether a gene or the product encoded by the gene is essential to the function of the cell can be performed, in the example bacteria, using methods including, but not limited to, gene disruption in E. coli (Datsenko et al., 2000, Proc. Natl. Acad. Sci. USA 97: 6640-5; Murphy, 1998, J. Bacteriol. 180: 2063-71; and Winans et al., J. Bacteriol 161: 1219-21), pKO3 method (Link et al., 1997, J. Bacteriol. 179: 6228-37), genomics (Arigoni et al., 1998, Nat. Biotechnol. 16(9): 851-6), GAMBIT (Akerley et al., Proc. Natl. Acad. Sci USA 95: 8927-32) and gene disruption in S. pneumoniae (Lee et al., 1999 Appl. Environ. Microbiol. 65: 1883-90 and Lee et al., 1998, Appl. Environ Microbiol. 64: 4796-802).

One embodiment of the invention is to screen known essential genes using the process(es) set forth to identify new compounds which modulate or perturb these essential genes. As new essential genes are identified, these genes can similarly be screened for compounds which modulate the activity of the gene. This modulation of gene activity can occur at the level of gene transcription into mRNA, mRNA translation into the protein encoded by the gene or most preferred, modulation of the activity of the protein encoded by the gene.

3. Target Prediction

Predicting the molecular target of a particular drug will comprise the biostatistical assessment or weighting of the data obtained by preferably at least three different methods 110-125 to determine which gene or genes (or gene product) are being modulated or perturbed by a particular drug. In a less preferred embodiment, the predicted drug target(s) identified by at least two methods can be analyzed to determine the putative target(s) of a drug. The three preferred methods of obtaining gene target information for gene target prediction are based on (1) transformation selection 110, (2) compound-specific transcription profiles by microarray 115, and (3) resistance mutation mapping 120. To further enhance the results of these three methods, additional methods 125 such as a three hybrid screening assay, metabolic profiling and/or proteomic profiling. Additional embodiments include any combination of at least two or more of the assays listed herein.

The information obtained from each of these at least two assays may then be weighted to determine from the list of potential targets (e.g., mRNAs) identified by each of the assays individually, which gene product or gene products is the predicted target of the drug that perturbed or modulated the activity of the microorganism or cell to which the drug had been administered.

Once the molecular target is predicted (see FIGS. 12 and 13), the preferred embodiment of the invention further comprises a step of validation or verification of the gene target. The validation portion of a GARIT technique 100 also confirms a compound's ability to target the molecular target. This validation step comprises using one of two methods (145 and 155), although both methods can be performed. The step of validation is discussed greater detail below.

3.1 Transformation Selection

The first independent target prediction process that can be used is Transformation Selection 1 10. When using the Transformation Selection Process 110, the inhibitory activity of the compound is tested against cells which are over-expressing a variety of bacterial genes. Overexpression of the gene encoding the drug target or of genes encoding proteins involved in other steps in the same pathway is known to rescue cells from the drug in many cases (e.g., Cácares et al., 1997, J. Bacteriol. 179: 5046-55; Rine et al., 1983, Proc. Natl. Acad. Sci. USA 80: 6750-4). Overexpression can be achieved by integration into the genome of a cell a single copy of each gene linked to a strong promoter, but preferably over-expression is achieved by transforming a cell culture with multicopy extrachromosomal plasmids carrying the genes linked to a strong promoter. In the most favorable form of the test, a library of genes or gene fragments is built in the vector of choice, introduced into a culture of the microbial cells by transformation, and resulting transformed cells are selected for those which are resistant to the compound of interest. Preferred embodiments are exemplified in Examples 1 and 2 below. The plasmids are isolated from several such resistant cells or the gene(s) inserted in the plasmids is amplified from whole cells by PCR, and the identity of the gene(s) which conferred resistance is determined by DNA sequencing. In most cases, a gene product (e.g., a protein) is the molecular target of the drug or is in the same cellular pathway as the molecular target.

3.1.1 Overexpression of the Genes

Plasmids overexpressing the genes encoding a putative molecular target are placed into the microorganisms. Then, the microorganisms are grown under suitable conditions but in the presence of the drug to which they are sensitive. Cells are then identified which carry a plasmid that rescues them from effects of the drug. Identification of microorganisms carrying a plasmid capable of rescuing them from the effects of the a drug or composition can be carried out in liquid culture, preferably in microcultures in standard 96- or 384-well microtiter dishes for high-throughput screening. The method may be applied to identify genes, which when overexpressed, provide resistance to each compound or composition known to inhibit cell growth or viability.

At least two sources of overexpression plasmids are feasible. In one embodiment, a small focused library can be generated. For example, each of the putative targets validated as broad spectrum or missing in only one G⁺ or G⁻ species by comparative genomics applied to predicted genes from microbial genomic sequences may be amplified by PCR and inserted into a multi-copy expression plasmid to create a mini-library, in for example, B. subtilis or other microorganism. Preferably, such putative targets may previously have been validated by gene knockout experiments as essential genes. For example, as assays are run and predictions of molecular target-drug combinations and essential genes or gene products determined, this information is accumulated and stored in a database. Such databases which accumulate the data as it is discovered allows the scientist to rapidly determine whether a certain gene is known to be essential or nonessential or whether it is uncharacterized, without having to assay it before hand. Alternatively, the database can also accumulate information regarding compounds and which genes or gene products they target, as the information is obtained. Similarly, such drug information may be used in screening homologs, analogs, mimetics or derivatives of drugs in an effort to find more efficacious agents which target a specific gene or gene product.

When rescue from the inhibitory effects of an active compound is achieved with a member of this plasmid library, then the identity of the likely target may be discovered by sequencing a portion of the insert. Alternatively, the 200 plasmids may be stored in known positions in a microtiter dish, used to transform the bacterial host strain, and plated on drug-containing agar plates in the same positions. Then, the identity of the rescuing plasmid is identified simply from its position.

In another embodiment, an expression library consisting of all predicted coding “open reading frames,” or ORFs can be generated. Overexpression plasmids can be identified using an expression library consisting of all predicted coding ORFs in a microorganism (e.g., E. coli) generated as a multicopy expression plasmid library by cloning PCR products representing all of the coding ORFs into an expression plasmid. “ORFmer” PCR primer pairs designed to amplify, for example, all E. coli genes, can be obtained from Sigma GenoSys, Inc. Such PCR products of all coding ORFs can further be utilized to produce, for example, an E. coli microarray as discussed in Section 3.2 below. For additional discussion on this technique, see, e.g., C. S. Richmond et al., 1999, Nuc. Acids Res. 27: 3821-3835.

These libraries can be used to transform a host microorganism, such as E. coli, and the resulting transformants plated on agar plates or in multi-well microtiter plates containing a compound of interest. The identity of the gene or genes providing resistance to the compound or composition may then be determined by sequencing a portion of the plasmid insert.

3.1.2 Regulatory elements

In one embodiment of the invention, preferably an essential gene or a gene believed to be essential to the survival of the organism or cell is regulated by fusion of the gene, or a biologically active portion thereof, to a heterologous regulatory element. A heterologous regulatory element is one that is not normally associated with, and does not normally regulate, the gene which it regulates as practiced in the invention. Regulatory elements can comprise transcriptional, post-transcriptional, translational, and post-translational elements, and elements related to replication. Examples of regulatory elements include promoters, enhancers, operators and elements that modulate the rate of transcription initiation, elongation and/or termination. Post-transcriptional regulatory elements can include those influencing messenger stability, processing and transport. Translational regulatory elements include those which modulate the frequency of translation initiation and the rate of translational elongation. Post-translational regulatory elements can include those which influence protein processing, stability and transport. Replication-associated regulatory elements can include those related to gene dosage.

Preferred embodiments of the transformation selection step use a regulatable promoter, such as the araBAD promoter (e.g., P_{BAD}). Regulation using P_{BAD} is discussed in, Schleif, 1992, Ann. Rev. Biochem. 61: 199-223; Guzman et al., 1995, J. Bacteriol. 177: 4121-30; and Gallegos et al., 1997, Microbiol. & Mol. Biol. Rev. 61 393-410. The AraC/P_{BAD} regulatory system allows very low basal levels of transcription. The P_{BAD} promoter is regulated by the AraC protein, which has both positive and negative regulatory activities. In the absence of L-arabinose or other inducers (such as, for example, L-ribose), AraC represses transcription from P_{BAD} by binding to sites upstream of the P_{BAD} transcription initiation site. The activity of P_{BAD} is directly proportional to the concentration of arabinose in the environment, and, at low arabinose concentrations, very low levels of gene expression can be obtained.

3.1.3 Construction of Gene Fusions

In a preferred embodiment, fusion of a heterologous regulatory element to a gene encoding protein preferably of an essential cellular function is accomplished by insertion of an ara regulatory cassette into the chromosome of the organism or cell under study, or insertion of the cassette into a plasmid. The ara regulatory cassette can include nucleic acid molecule comprising, in the following order, the AraC gene, PC (the araC promoter) and P_{BAD} (the promoter regulating expression of the araB, araA, and araD genes). This is the order in which these elements are arranged on the E. coli and S. typhimurium chromosomes, in which the PC and P_{BAD} promoters are adjacent. Insertion of the cassette will provide AraC function to the cell and place downstream coding sequences under the control of P_{BAD}.

In addition to the P_{BAD} promoter, other promoters from the AraC/XylS family, from any prokaryotic or eukaryotic organism, can be utilized in the transformation selection assay. See, de Vos et al., 1997, Curr. Opin. Biotechnol. 8: 547-53; Kleerebezem et al., 1997, Mol. Microbiol. 24: 895-904. Additional suitable regulatory systems include the malM/malX system of S. pneumoniae, regulated by MalR (WO 99/52926; Stassi et al., 1982, Gene 20: 359-66; Nieto et al., 1997, J. Biol. Chem. 272: 30860-5), the raf regulatory system of Streptococcus pneumoniae (see WO 99/52926), the P_{AGA} promoter system (see WO 99/52926), P_{lac/ara-1} and P_{tetO-1} (Lutz et al., 1997, Nuc. Acids Res. 25: 1203-10)P_{spac} (Yansura et al., 1984, Proc. Natl. Acad. Sci. USA 81:439-443), P_{rhaBAD}, P_{LtetO-1}, P_{lac}, P_{trc}, Pₜᵣₚ, λP_{L} and P_{tetA}. Preferred promoters in gram-positive bacteria for use in the transformation selection assay include P_{xyl-tet01}, P_{spac}, P_{nisA}, P_{araE} and P_{xylA}.

Preferred regulators include, but are not limited to, L-arabinose, isopropyl-β-thiogalactopyranoside (IPTG), anhydrous tetracycline and xylose.

The genes to be studied using the transformation selection assay typically are placed in operative linkage with one or more regulatory sequences. However, one or more coding sequences can be operatively linked to one or more regulatory sequences. An operator is considered to be operatively linked to a promoter or to a coding sequence if binding of a repressor to the operator inhibits initiation at the promoter so as to prevent or diminish expression of the coding sequence. An operably linked transcriptional regulatory sequence is generally joined in cis with the coding sequence, but may not be located immediately adjacent to it.

The recombinant constructs contemplated can exist as freely-replicating extrachromosomal elements, such as plasmids or episomes, but preferably exist in the form of chromosomal recombinants. Methods for obtaining chromosomal integration of recombinant constructs are known, and have been described, for example in, Gerhardt et al., METHODS FOR GENERAL AND MOLECULAR MICROBIOLOGY (American Society for Microbiology, 1994); Link et al., 1997, J. Bacteriol. 179: 6228-37; and Metcalf et al., 1996, Plasmid 35: 1-13.

Introduction of the construct into a host cell is performed by methods that are well known in the art, including for example, natural or artificial transformation, transduction, conjugation, microinjection, transfection, electroporation, CaPO₄ coprecipitation, DEAE-dextran, lipid mediated transfer, etc.

The host cells to be studied by transformation selection are cultured in any suitable growth medium, which includes liquid and solid media. Appropriate media for growth can be found, for example in, BERGEY'S MANUAL OF SYSTEMATIC BACTERIOLOGY, vol. 2 (Williams & Wilkins, 1980); Gerhardt et al., METHODS FOR GENERAL AND MOLECULAR MICROBIOLOGY (American Society for Microbiology, 1994); and Freshney, CULTURE OF ANIMAL CELLS: A MANUAL OF BASIC TECHNIQUE, 3^{rd} ed. (Wiley-Liss, 1994).

3.1.4. Screening for Gene Underexpression

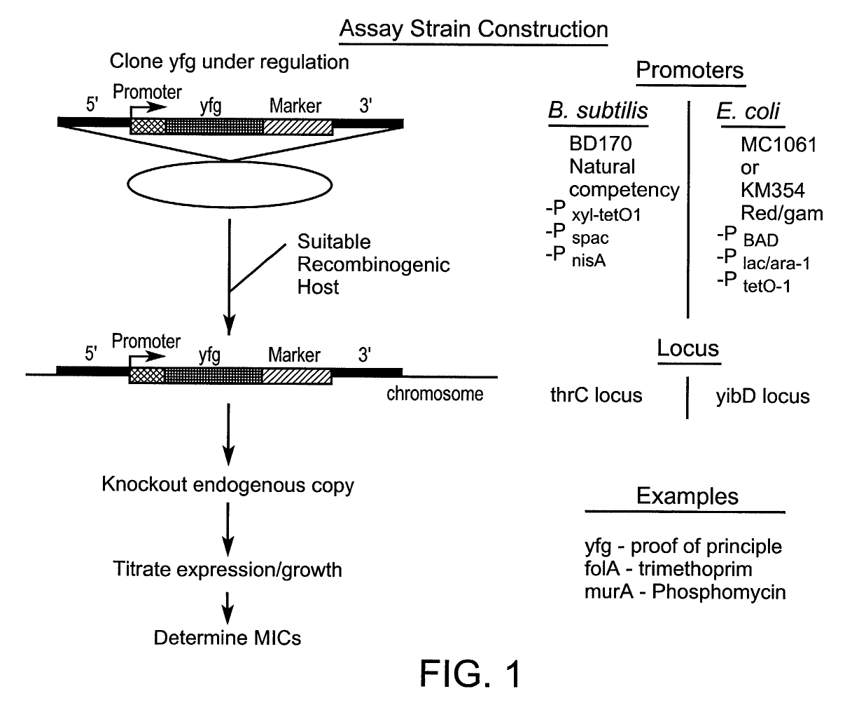

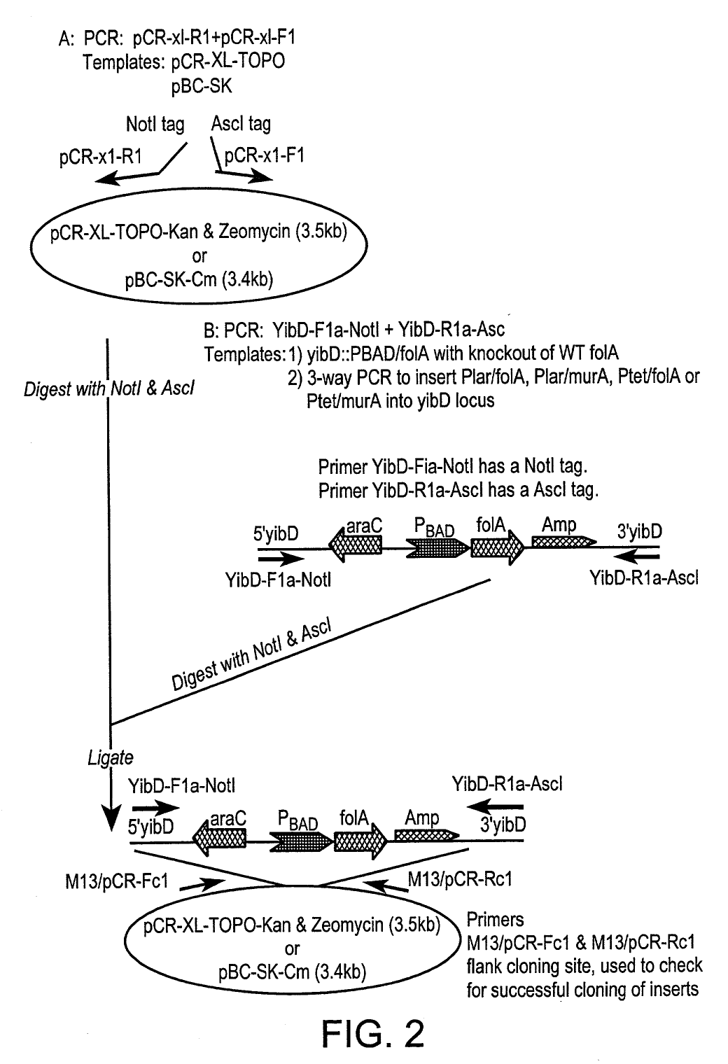

The essential genes are placed in a vector wherein a hypersensitivity or resistence cassette is operably linked to other genetic elements (e.g., selectable markers, ori, etc.). This process is used at the Transformation Selection Process 110 and the Gene Expression 155 steps of the GARIT technique 100. The vector further can comprise at least one multiple cloning site (MCS) and at least one antibiotic resistance gene, (such as genes conferring resistance to ampicillin, carbenicillin, chloramphenicol, kanamycin, streptomycin, spectinomycin, gentamicin, phleomycin and tetracycline) or other selectable marker. In general, any intergenic region or non-essential gene that can be disrupted without affecting the expression of an essential gene can be used as a locus for regulated ectopic expression of a gene of interest. In a preferred construct for E. coli and other gram-negative bacteria, the essential gene is placed in the vector flanked by homologous DNA 5′ and 3′ to the yibD locus. The thrC locus is a preferred site for use in gram-positive bacteria such as B. subtilis; the essential gene is flanked by homologous DNA 5′ and 3′ to the thrC locus. For gram-negative and gram-positive bacteria, the schema in FIG. 1 can be used. The endogenous copy of the essential gene can be knocked out after the inducible copy of the essential gene is introduced into the cell. Universal cloning templates can be created according to the schema presented in FIG. 2. Cloning procedures can also be performed as known to the skilled artisan, or as described in the art.

Once a suitable vector comprising an inducible copy of the essential gene is assembled, the vector is transformed or transfected into competent host cells, such as gram-negative or gram-positive bacteria. Transformed colonies that have been selected for resistance to an antibiotic specified by the resistance cassette carried by the vector, are preferably assessed to determine whether they carry one copy of the integrated construct in the correct locus, which can be performed by polymerase chain reaction (PCR). The endogenous copy of the essential gene is then deleted from colonies containing the inducible form of the essential gene using standard allelic replacement procedure, in which the essential gene is replaced by an antibiotic resistance gene. Transformants carrying a deletion of the essential gene are selected in the presence of the regulator and the antibiotic specified by the antibiotic resistance cassette. The resulting colonies can then again be verified by diagnostic PCR for deletion of the wild-type form of the essential gene. Appropriate DNA primers for use in verifying the presence of the inducible form and the lack of the wild-type form of the essential gene can be created as necessary. In the final strain, only the inducible form of the essential gene should be expressed by the recombinant cell, leading to a strain that is dependent on the presence of regulator for growth. Alternatively, the endogenous promoter of the essential gene of interest may be replaced with a regulatable promoter in situ to confer regulatability on the essential gene without the need to add a second copy of the essential gene and delete the wild-type copy.

Prior to screening chemical agents against the assay strain, the amount of regulator required for growth of the recombinant hosts is titrated. To determine the influence of a compound or composition, the compound or composition is screened across the recombinant cell expressing the inducible essential gene and the minimal inhibitory concentration (MIC) for each compound is determined.

The MIC assay can be performed as described in the examples provided as would be readily known in the field. The MIC assay can be utilized with either essential gene vectors prepared for underexpression or for overexpression of the gene to determine whether regulated expression of the essential gene affects the MIC for a given chemical compound. Alternatively, a library of chemical compounds can be directly screened against the recombinant host expressing the inducible essential gene at the minimal level required for growth.

The resistance cassettes,vectors, and preparation and strategy discussed above and which was utilized in Examples 1 and 2 for underexpressing essential genes, can also be utilized for over-expressing essential genes.

3.2 Compound-Specific Transcription Profiles by Microarray 115

A second independent target prediction process which can be used in a GARIT technique 100, is Gene Expression by Microarray 115. The principle of this approach is the anticipation that each compound or composition which blocks cell growth and/or viability will cause a transcriptional response by the cell exposed to the compound or composition. This response will then reveal the cellular pathway effected by the compound. This method would be true for most drugs. See for example discussion of the influence of isoniazid on M. tuberculosis by Wilson et al., 1999, Proc. Natl. Acad. Sci. USA 96: 12833-8 as an example.

With microarrays, the effect of the inhibitory compound on expression of genes in the microbial cell is determined by means of hybridization of labeled probe prepared from RNA onto a microarray containing a substantial fraction of the cell's genes. For example, microbial cells are grown in the presence of sub-inhibitory concentrations of each of the inhibitory compounds and/or cells are grown for only a few generations in the presence of a fully inhibitory concentration of each of the compounds. Total RNA is prepared from the compound-treated cells and from cells grown in the absence of the compound. The RNA preparations are labeled differentially by standard methods for use as probes to the same microarray (see, e.g., Wilson et al., 1999, Proc. Natl. Acad. Sci. USA 96: 12833-8; Tao et al., 1999, J. Bacteriol. 181: 6425-40; and Richmond et al., 1999, Nuc. Acids Res. 27: 3821-35). The signals from each of the probes are processed as is known in the art to reduce most background and spurious signals and converted into ratios indicating which genes exhibited increased or decreased expression during drug treatment as compared to growth in the absence of the drug. The identity of the genes whose expression level increased or decreased when cells were incubated in the presence of a drug as compared to when cells were grown without drug provides evidence for the mode of action and/or the specific molecular target of the drug. Clustering algorithms may be applied by means of specific software to identify the group of genes which behave similarly in different experiments with the same drug and control combinations, or they may be applied to several experiments with different drug and control combinations to reveal which drug treatments cluster together. Knowledge that gene expression measurements taken from cells treated with a drug of unknown mode of action cluster most closely with gene expression measurements taken from cells treated with a drug of known mode of action provides evidence that the two drugs are working through similar or identical modes of action and/or act on the same molecular target in the microbe. This information can also be accumulated in a database as it is obtained, and be used to further characterize drug and/or gene action.

The benefit of utilizing a microarray is that it will identify many genes/proteins that are potentially perturbed by the administration of a particular agent. This is because the use of the microarray will allow the measurement of the transcriptional state of a cell, and even though the agent may act on a post-transcriptional mechanism (e.g., inhibiting protein activity or inhibiting protein degradation), the administration of an agent which perturbs a microorganism will almost always exert direct or at least indirect effects on the transcriptional state of the microorganism.

The microarrays are used in the GARIT technique 100 in both the prediction step 115 and in the validation step 155. Methods of preparing the microarrays are discussed generally below.

3.2.1 Microarray Preparation

These arrays are employed for analyzing the transcriptional state of a cell and especially for comparing the impact on a microorganism exposed to a drug of interest. In one embodiment, transcript arrays are produced by hybridizing detectably labeled polynucleotides representing the mRNA transcripts present in a cell (e.g., fluorescently labeled cDNA synthesized from total cell mRNA) to a microarray. A microarray is a surface with an ordered array of binding (e.g., hybridization) sites for products of many of the genes in the genome of a cell or organism, preferably most or almost all of the genes. Microarrays can be made in a number of ways, of which several are described below. However produced, microarrays share certain characteristics. The arrays are reproducible, allowing multiple copies of a given array to be produced and easily compared with each other. Preferably the microarrays are small, usually smaller than 5 cm², and they are made from materials that are stable under binding (e.g. nucleic acid hybridization) conditions. A given binding site or unique set of binding sites in the microarray will specifically bind the probe product of a single gene in the cell.

For example, when cDNA complementary to the RNA of a cell is made and hybridized to a microarray under suitable hybridization conditions, the level of hybridization to the site in the array corresponding to any particular gene will reflect the prevalence in the cell of mRNA transcribed from that gene. For example, when detectably labeled (e.g., with a fluorophore) cDNA complementary to the total cellular mRNA is hybridized to a microarray, the site on the array corresponding to a gene (i.e., capable of specifically binding the product of the gene) that is not transcribed in the cell will have little or no signal (e.g., fluorescent signal), and a gene for which the encoded mRNA is prevalent will have a relatively strong signal.

In other embodiments, cDNAs from two different cells are hybridized to the binding sites of the microarray. For drug response analysis, one cell is exposed to a drug and another cell of the same type is not exposed to the drug. The cDNA derived from each of the two cell types are differently labeled so that they can be distinguished. In one embodiment, for example, cDNA from a cell treated with a drug is synthesized using a fluorescein-labeled dNTP, and cDNA from a second cell not exposed to drug is synthesized using a rhodamine-labeled dNTP (other fluorophores can also be utilized). When the two cDNAs are mixed and hybridized to the microarray, the relative intensity of signal from each cDNA set is determined for each site on the array, and any relative difference in abundance of a particular mRNA detected.

For instance, the cDNA from the drug-treated cell will fluoresce green when the fluorophore is stimulated and the cDNA from the untreated cell will fluoresce red. As a result, when the drug treatment has no effect, either directly or indirectly, on the relative abundance of a particular mRNA in a cell, the mRNA will be equally prevalent in both cells and, upon reverse transcription, red-labeled and green-labeled cDNA will be equally prevalent. When hybridized to the microarray, the binding site(s) for that species of RNA will emit wavelengths characteristic of both fluorophores (and appear brown in combination). In contrast, when the drug-exposed cell is treated with a drug that, directly or indirectly, increases the prevalence of the mRNA in the cell, the ratio of green to red fluorescence will increase. When the drug decreases the mRNA prevalence, the ratio will decrease.

The use of a two-color fluorescence labeling and detection scheme to define alterations in gene expression has been described, e.g., by Shena et al., 1995, Science 270: 467-70. An advantage of using cDNA labeled with two different fluorophores is that a direct and internally controlled comparison of the mRNA levels corresponding to each arrayed gene in two cell states can be made, and variations due to minor differences in experimental conditions (e.g., hybridization conditions) will not affect subsequent analyses. However, it will be recognized that it is also possible to use cDNA from a single cell, and compare, for example, the absolute amount of a particular mRNA in, e.g., a drug-treated or pathway-perturbed cell and an untreated cell.

Microarrays are known in the art and consist of a surface to which probes that correspond in sequence to gene products (e.g., cDNAs, mRNAs, cRNAs, polypeptides, and fragments thereof), can be specifically hybridized or bound at a known position. In one embodiment, the microarray is an array (i.e., a matrix or a bead) in which each position represents a discrete binding site for a product encoded by a gene (e.g., a protein or RNA), and in which binding sites are present for products of most or almost all of the genes in the organism's genome. In a preferred embodiment, the “binding site” (hereinafter, “site”) is a nucleic acid or nucleic acid analogue to which a particular cognate cDNA can specifically hybridize. The nucleic acid or analogue of the binding site can be, e.g., a synthetic oligomer, a full-length cDNA, a less-than full length cDNA, or a gene fragment.

Although preferredly the microarray contains binding sites for products of all or almost all genes in the target organism's genome, such comprehensiveness is not necessarily required for the analysis contemplated herein. Usually the microarray will have binding sites corresponding to at least about 50% of the genes in the genome, often at least about 75%, more often at least about 85%, even more often more than about 90%, and most often at least about 99%. Preferably, the microarray has binding sites for genes relevant to the action of a drug of interest or in a biological pathway of interest. A “gene” is identified as an open reading frame (ORF) of preferably at least 50, 75, or 99 nucleic acids or more from which a messenger RNA is transcribed in the organism (e.g., if a single cell) or in some cell, in a multicellular organism. The number of genes in a genome can be estimated from the number of mRNAs expressed by the organism, or by extrapolation from a well-characterized portion of the genome. When the genome of the organism of interest has been sequenced, the number of ORFs can be determined and mRNA coding regions identified by analysis of the DNA sequence.

3.2.1.1 Preparing Nucleic Acids for Microarrays

As noted above, the “binding site” to which a particular cognate cDNA specifically hybridizes is usually a nucleic acid or nucleic acid analogue attached at that binding site. In one embodiment, the binding sites of the microarray are DNA polynucleotides corresponding to at least a portion of each gene in an organism's genome. These DNAs can be obtained by, e.g., polymerase chain reaction (PCR) amplification of gene segments from genomic DNA, cDNA (e.g., by RT-PCR), or cloned sequences. PCR primers are chosen, based on the known sequence of the genes or cDNA, that result in amplification of unique fragments (i.e. fragments that do not share more than 10 bases of contiguous identical sequence with any other fragment on the microarray). Computer programs are useful in the design of primers with the required specificity and optimal amplification properties.

In the case of binding sites corresponding to very long genes, it will sometimes be desirable to amplify segments near the 3′ end of the gene so that when oligo-dT primed cDNA probes are hybridized to the microarray, less-than-full length probes will bind efficiently. Typically each gene fragment on the microarray will be between about 50 bp and about 2000 bp, more typically between about 100 bp and about 1000 bp, and usually between about 300 bp and about 800 bp in length. PCR methods are well known and are described, for example, in Innis et al., eds., PCR PROTOCOLS: A GUIDE TO METHODS AND APPLICATIONS (Academic Press Inc. 1990). The number of nucleic acid sequences on an array can range between 1,000 and 100,000, more often between 5,000 and 50,000.

An alternative means for generating the nucleic acid for the microarray is by synthesis of synthetic polynucleotides or oligonucleotides, e.g., using N-phosphonate or phosphoramidite chemistries (Froehler et al., 1986, Nuc. Acid Res. 14: 5399-5407; McBride et al., 1983, Tetrahedron Lett. 24: 245-8). Synthetic sequences are between about 15 and about 500 bases in length and more typically between about 15 and about 50 bases. Nucleic acid analogues may also be used as binding sites for hybridization. An example of a suitable nucleic acid analogue is peptide nucleic acid (see, e.g., Egholm et al., 1993, Nature 365: 566-8).

In an alternative embodiment,the binding (hybridization)sites are made from plasmid or phage clones of genes, cDNAs (e.g., expressed sequence tags), or inserts therefrom (Nguyen et al., 1995, Genomics 29: 207-9). In yet another embodiment, the polynucleotide of the binding sites is RNA.

3.2.1.2 Attaching Nucleic Acids to the Solid Surface

The nucleic acid or analogue are attached to a solid support, which may be made from glass, plastic (e.g., polypropylene, nylon), polyacrylamide, nitrocellulose, or other materials. A preferred method for attaching the nucleic acids to a surface is by printing on glass plates, as is described generally by Schena et al., 1995, Science 270: 467-70. This method is especially useful for preparing microarrays of cDNA. See also DeRisi et al., 1996, Nature Genetics 14: 457-60; Shalon et al., 1996, Genome Res. 6: 639-45; and Schena et al., 1996, Proc. Natl. Acad. Sci. USA 93: 10614-9.

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Science 251: 767-73; Pease et al., 1994, Proc. Natl. Acad. Sci. USA 91: 5022-6; Lockhart et al., 1996, Nature Biotech. 14: 1675; U.S. Pat. Nos. 5,578,832; 5,556,752; and 5,510,270) or other methods for rapid synthesis and deposition of defined oligonucleotides (Blanchard et al., 1996, Biosensors & Bioelectronics 11: 687-90). When these methods are used, oligonucleotides (e.g., 20-mers) of known sequence are synthesized directly on a surface such as a derivatized glass slide. Usually, the array produced is redundant, with several oligonucleotide molecules per RNA.

Other methods for making microarrays, e.g., by masking (Maskos et al., 1992, Nuc. Acids Res. 20: 1679-84), may also be used. In principal, any type of array, for example, dot blots on a nylon hybridization membrane (see Sambrook et al., MOLECULAR CLONING—A LABORATORY MANUAL (2nd ed.), Vol. 1-3, Cold Spring Harbor Laboratory, Cold Spring Harbor, N.Y., 1989, which is incorporated in its entirety for all purposes), could be used, although, as will be recognized by those of skill in the art, very small arrays will be preferred because hybridization volumes will be smaller.

3.2.1.3 Generating Labeled Probes

Methods for preparing total and poly(A)⁺RNA are well known and are described generally in Sambrook et al., (1989). Since most bacterial mRNA lacks poly(A) tracts or contains only very short poly(A) tracts, total RNA is typically isolated from bacteria. In one embodiment, RNA is extracted from cells of the various types of interest in this invention using guanidinium thiocyanate lysis followed by CsCl centrifugation (Chirgwin et al., 1979, Biochemistry 18: 5294-9). Poly(A)⁺. Cells of interest include wild-type cells, drug-exposed wild-type cells, modified cells (e.g., recombinant underexpressing cells of Section 4.0), and drug-exposed modified cells.

Labeled cDNA is prepared from mRNA by oligo dT-primed or, for bacterial mRNA by random-primed reverse transcription, both of which are well known in the art (see e.g., Klug et al., 1987, Meth. Enzymol. 152: 316-25). Reverse transcription may be carried out in the presence of a dNTP conjugated to a detectable label, most preferably a fluorescently labeled dNTP. Alternatively, isolated mRNA can be converted to labeled antisense RNA synthesized by in vitro transcription of double-stranded cDNA in the presence of labeled dNTPs (Lockhart et al., 1996, Nature Biotech. 14: 1675-80). The cDNA or RNA probe also can be synthesized in the absence of detectable label and may be labeled subsequently, e.g., by incorporating biotinylated dNTPs or rNTP, or some similar means (e.g., photo-cross-linking a psoralen derivative of biotin to RNAS), followed by addition of labeled streptavidin (e.g., phycoerythrin-conjugated streptavidin) or the equivalent.

When fluorescently-labeled probes are used, many suitable fluorophores are known, including fluorescein, lissamine, phycoerythrin, rhodamine (Perkin Elmer Cetus), FluorX (Amersham), Cy3-dUTP or Cy5-UTP (Amersham) and others (see, e.g., Kricka, 1992, NONISOTOPIC DNA PROBE TECHNIQUES, Academic Press San Diego, Calif). It will be appreciated that pairs of fluorophores are chosen that have distinct emission spectra so that they can be easily distinguished. The microarrays can also be performed as described below in Example 4.

3.2.1.4 Hybridization to Microarrays

Nucleic acid hybridization and wash conditions are chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls (see, e.g., Shalon et al., 1996).

Optimal hybridization conditions will depend on the length (e.g., oligomer versus polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., (1989); Ausubel et al., CURRENT PROTOCOLS IN MOLECULAR BIOLOGY (Greene Publishing and Wiley-Interscience 1995), Shena et al., 1996, Proc. Natl. Acad. Sci. USA 93: 10614-9; Tijessen, 1993, Hybridization With Nucleic Acid Probes (Elsevier Science Publishers, 1993); and Kricka, NONISOTOPIC DNA PROBE TECHNIQUES (Academic Press, 1992).

3.2.1.5 Signal Detection and Data Analysis

When fluorescently labeled probes are used, the fluorescence emissions at each site of a transcript array can be, preferably, detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (Shalon et al., 1996, Genome Res. 6: 639-45). Preferredly,the arrays are scanned with a laser fluorescent scanner with a computer controlled X-stage and a microscope objective. Sequential excitation of the two fluorophores is achieved with a multi-line, mixed gas laser and the emitted light is split by wavelength and detected with two photomultiplier tubes. Alternatively, the fiber-optic bundle described by Ferguson et al., 1996, Nature Biotech. 14: 1681-4, may be used to monitor mRNA abundance levels at a large number of sites simultaneously.

Signals are recorded and, in a preferred embodiment, analyzed by computer, e.g., using a 12-bit analog to digital board. In one embodiment, the scanned image can be despeckled using graphics program such as, Hijaak Graphics Suite and then analyzed using an image gridding program that creates a spreadsheet of the average hybridization at each wavelength at each site. If necessary, an experimentally determined correction for “cross talk” (or overlap) between the channels for the two fluorophores may be made. For any particular hybridization site on the transcript array, a ratio of the emission of the two fluorophores can be calculated. The ratio is independent of the absolute expression level of the cognate gene, but is useful for genes whose expression is significantly modulated by drug administration, gene deletion, or any other tested event.

According to the method of the invention, the relative abundance of an mRNA in two cells or cell lines is scored as a perturbation and its magnitude determined (i.e., the abundance is different in the two sources of mRNA tested), or as not perturbed (i.e., the relative abundance is the same). As used herein, a difference between the two sources of RNA of at least a factor of about 25% (e.g., RNA from one source is 25% more abundant in one source than the other source), more usually about 50%, even more often by a factor of about 2 (twice as abundant), 3 (three times as abundant) or 5 (five times as abundant) is scored as a perturbation. Present detection methods allow reliable detection of difference of an order of about 2-fold to about 10-fold.

Preferably, in addition to identifying a perturbation as positive or negative, it is advantageous to determine the magnitude of the perturbation. This is performed by calculating the ratio of the emission of the two fluorophores used for differential labeling, or by analogous methods that will be readily apparent to those of skill in the art.

3.2.1.6 Applying Microarrays to Drug Analysis

Use of microarrays in either the prediction or validation arms of the invention is useful for both determining the molecular target as well as the impact of a drug on a gene or genes or a gene product(s).

In the target prediction arm of the invention, the microarray is utilized to determine what gene expression pattern(s) are perturbed when a putative drug is administered to a microorganism. For example, provided with a candidate drug that appears to affect a putative biological pathway, the methods of the present invention can be applied to confirm that the putative pathway is indeed a pathway of action of the drug, as well as for development of drugs (e.g., such as an ideal drug) that are more specific for the putative pathway (i.e., are more pathway-specific) in that they affect fewer biological pathways besides the desired putative pathway.

The microarray component is performed by: (i) measuring drug response data for the drug or candidate compound or agent of interest; (ii) measuring the perturbation response for the putative biological pathway of drug action (e.g., if the biological pathway originates at a gene, then the expression of the gene may be controlled in a graded manner and the response observed); (iii) representing the drug response data as clearly as possible in terms of response in a particular putative pathway of drug action; and (iv) assessing whether significant effects of the drug have been fully identified.

Verification or validation of the molecular target is conducted by preparing a microorganism in which the native gene is functionally knocked out and a regulatable form of the gene as prepared by the method described in Section 4.0 below is inserted into the microorganism being studied. The gene expression profile as determined by a microarray experiment of this recombinant microorganism grown under conditions in which the regulatable gene is underexpressed is then compared to the gene expression profile determined by microarray of the wild-type microorganism to which the drug had been administered. If the two gene expression profiles yielded a similar matrix of up-regulated and down-regulated genes, wherein the degree of up-regulation and down-regulationis preferably not different as between the two arrays by more than a factor of four, and preferably by less than a factor of three, then the drug target has been confirmed.

If the drug response data thus obtained surpasses a significance thresh-hold of at least 90% or greater (preferably 95% or greater), then this indicates that the candidate drug is highly specific for the putative biological pathway (with few or no direct effects on other biological pathways, such as those originating at other genes, or gene products, or gene product activities). If the correlation between the two gene expression profiles is found to be less than 90% significant, then other biological pathways or genes are likely to be the target of the candidate agent, if any target exists at all.

In the latter case, in which other biological pathways in the cell are affected, the drug structure may be modified (e.g., using organic synthesis methods well known in the arts of pharmaceutical or medicinal chemistry) or closely related compounds may be identified (homologs), or the like, and tested according to the present invention until a drug that is more target-specific (i.e., affecting fewer pathways or genes other than the putative pathway or gene or which has a greater affinity for the target gene or gene product) is identified.

Another object of the methods of this invention is to select, from a set of candidate compounds, the drug or drugs with the highest pathway specificity by identifying all the cellular biological pathways of compounds in the set. Usually, the drug with the highest pathway specificity will be the one that directly affects only its intended pathway. When the intended pathway or gene is not known, the drug that affects the fewest number of pathways or genes is likely to be more pathway-specific than a drug that affects a greater number of pathways or genes, and thus is a preferred candidate. A drug with high specificity (i.e., highly pathway-specific) is preferred because such a drug will likely have fewer side effects when administered to a patient.

In further applications, the invention can be used to identify the pathway(s) and genes upon which a drug acts, but for which the mechanism or pathway of action is not known. By identifying the pathway of action or gene upon which a drug acts with a desirable therapeutic activity, it is possible to identify other compounds having a similar therapeutic activity, as well as to identify compounds with greater pathway specificity. In such an application, drug response data is fit with a combination of pathways likely to be affected by the drug, or with pathways simply drawn from a database of characterized pathways, and the pathway combination best fitting the drug response determined. The methods of this invention also can be used to identify a compound or compounds that affect a previously identified biological pathway in a cell, or that affect a particular combination of pathways. In such an application, the significance of the best fit of the drug response data to the pathway response data (or combination of pathway response data) is determined to see if it meets a certain threshold of significance.

Another object of the invention is to identify biological pathways that mediate the therapeutic actions or that potentially induce side-effects of a drug of interest by comparison of the drug of interest with other drugs having similar therapeutic effects. Two drugs are considered to have similar therapeutic effects if they both exhibit similar therapeutic efficacy for the same disease or disorder in a patient or in an animal disease model. Drugs known to have similar, or closely similar, therapeutic affects are often found to act on the same biological pathways. Therefore, the methods of this invention can be applied to determine commonality of pathways and/or genes affected by the drug of interest and also of a second drug with similar therapeutic effects, as well as more fully characterize the second drug's potential in a patient (e.g., efficacy, potential side effects, etc.). This can be accomplished by comparing common pathways and genes determined for the additional drugs with similar therapeutic effects, to further characterize the therapeutic effects of the drug of interest.

For example, Staphylococcus epidermidis cells can be grown in the presence of various amounts of commonly used antibiotics. The dose of antibiotics can be at inhibitory, but sub-lethal levels. The cells can be grown for various lengths of time and then RNA prepared therefrom. The RNA is then differentially labeled from the compound treated cells as compared to normal cells and probed using a microarray representing each likely S. epidermidis gene. More than one experiment should be performed for a more accurate statistical analysis. After statistical processing of the data to subtract backgrounds and normalize the results to each experiment and between experiments, the data can be analyzed by means of software such as GeneSpring (Silicon Genetics, San Carlos, Calif.). Additional types of microarray analyzing software that can be employed include ARRAYSCOUT™ (LION Bioscience AG, Heidelberg, Germany) and GENESIGHT™ (BioDiscovery, Inc., Los Angeles, Calif.).

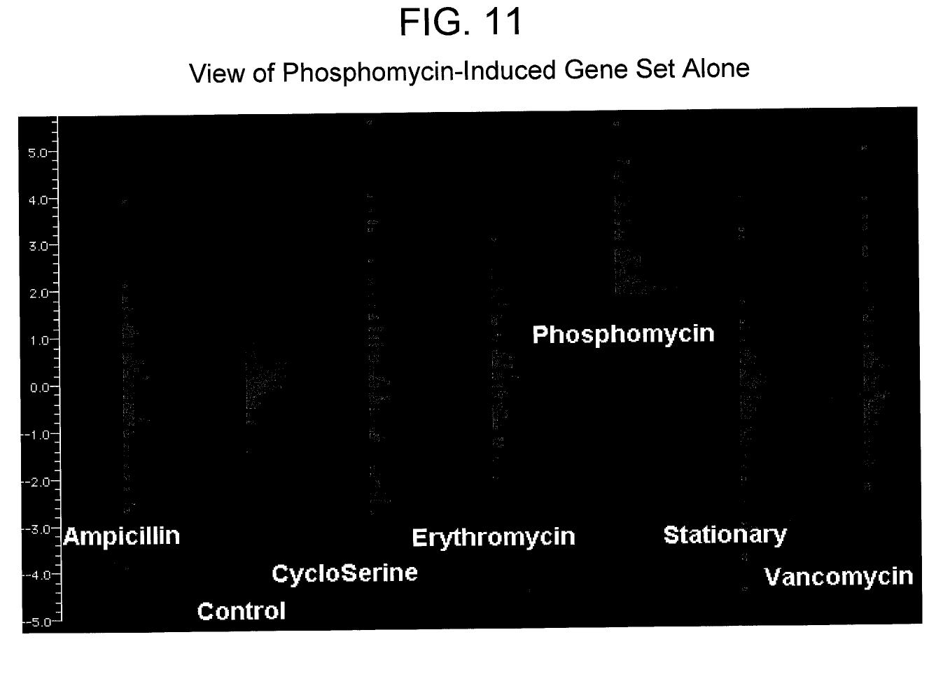

By highlighting the set of genes up-regulated in response to a candidate antibiotic administration and compared with the responses produced by the cell when exposed to other, known antibiotics to identify the genes whose expression is specifically perturbed by the candidate antibiotic or drug. Each drug treatment is expected to produce a relatively unique set of perturbed (up-regulated and down-regulated) genes (see, e.g., FIG. 11). These drugs act on different cellular targets and produce different transcriptional profiles. In most cases, the identity of the up-regulated genes will reveal the cellular pathway regulated by the drug. For example, ampicillin treatment of S. epidermidis cell is known to up-regulate the penicillin binding protein 2 gene (pbp2), a β-lactamase gene, and a regulator of β-lactamase gene expression. Similarly, as expression profiles are developed for other organisms and compounds, the information thus acquired can be added to known information and aid the prediction of new pathways and genes perturbed by the compound or composition.

3.3 Resistance Mutation Mapping 120

The third preferred independent target prediction process for use in the GARIT technique 100 of predicting gene targets involves resistance mutation mapping 120. In this step, cells which are resistant to the effects of the drug may be selected by methods known in the art, and the identity of the gene or genes in which these mutations reside may be determined by a variety of genetic mapping tools available in some species of bacteria and fungi. In some cases, the gene(s) which mutate and provide resistance to the drug are the direct molecular target of the drug (see, e.g., gyrA and parC as described by Fukuda et al., 1999, Antimicrobial Agents & Chemotherap. 43: 410-2; and rpoB as described by Severinov et al., 1993, J. Biol. Chem. 268: 14820-5). In other cases, especially in the case of complex molecular structures composed of multiple proteins, the genes which mutate and provide resistance in complex with the direct molecular target may be linked to that target through conformational changes (e.g., erythromycin; Chittum et al., 1994, J. Bacteriol. 176: 6192-8). At least knowledge of the identity of such genes defines the mode of action of the drug, if not the direct molecular target. In other cases, some genes which mutate and provide resistance encode enzymes which modify the target of the drug (e.g., ermK, Weisblum, 1995, Antimicrobial Agents & Chemotherap. 39: 577-85), and the identity of these genes reveals the target of the drug. Finally, in still other cases, genes which mutate and provide resistance may encode efflux pumps or drug modification enzymes (e.g., norA, see Neyfakh et al., 1993, Antimicrobial Agents & Chemotherap. 37: 128-9; and ampC, see Bou et al., 2000, Antimicrobial Agents & Chemotherap. 44: 428-32) and therefore provide little information regarding the mode of action or molecular target of the drug. These latter type of genes can usually be recognized by means of their high degree of sequence similarity to other members of those classes. One skilled in the art will recognize that these genes do not provide knowledge of the mode of action or the target of the drug.

3.3.1 Microorganisms

Bacterial strains suitable for study using mutation to resistance of GARIT include Gram-positive cocci such as Staphylococcus aureus, Streptococcus pyogenes, Streptococcus spp. (viridans group), Streptococcus agalactiae (group B), S. bovis, anaerobic forms of Streptococcus, Streptococcus pneumoniae, and Enterococcus spp.; Gram-negative cocci such as Neisseria gonorrhoeae, Neisseria meningitidis, and Branhamella catarrhalis; Gram-positive bacilli such as Bacillus anthracis, Bacillus subtilis, Corynebacterium diphtheriae and Corynebacterium species which are diptheroids (aerobic and anaerobic), Listeria monocytogenes, Clostridium tetani, Clostridium difficile, and other gram-negative species such as Escherichia coli, Enterobacter species, Proteus mirablis and other spp., Pseudomonas aeruginosa, Klebsiella pneumoniae, Salmonella, Shigella, Serratia and Campylobacter jejuni. Additional bacteria preferred for study using the resistance mutation mapping step are those which cause bacterial infections which result in diseases such as bacteremia, pneumonia, meningitis, osteomyelitis, endocarditis, sinusitis, arthritis, urinary tract infections, tetanus, gangrene, colitis, acute gastroenteritis, bronchitis, and a variety of abscesses, nosocomial infections,and opportunistic infections. A more comprehensive list of bacteria is provided in Table 1 and can be assessed using resistance mapping.

Preferred fungal organisms subjected to this step of the target identification methods include dermatophytes (e.g., Microsporum canis and other M spp.; and Trichophyton spp. such as T. rubrum, and T. mentagrophytes), yeasts (e.g., Candida albicans, C. Tropicalis, or other Candida species), Saccharomyces cerevisiae, Torulopsis glabrata, Epidermophyton floccosum, Malassezia furfur (Pityropsporon orbiculare or P. ovale), Cryptococcus neoformans, Aspergillus fumigatus, Aspergillus nidulans, and other Aspergillus spp., Zygomycetes (e.g., Rhizopus, Mucor), Paracoccidioides brasiliensis, Blastomyces dermatitides, Histoplasma capsulatum, Coccidioides immitis and Sporothrix schenckii.

The fungi contemplated for identifying drug and molecular targets using the described invention include those which cause fungal infections (mycoses) which are cutaneous, subcutaneous, or systemic in nature. Superficial mycoses include tinea capitis, tinea corporis, tinea pedis, onychomoycosis, perionychomycosis, pityriasis versicolor, oral thrush, and other candidoses such as vaginal, respiratory tract, biliary, esophageal, and urinary tract candidoses. Systemic mycoses include systemic and mucocutaneous candidosis, cryptococcosis, aspergillosis, mucormycosis (phycomycosis), paracoccidioidomycosis, North American blastomycosis, histoplasmosis, coccidioidomycosis, and sporotrichosis. Fungal infections also contribute to meningitis and pulmonary or respiratory tract diseases. Opportunistic fungal infections have proliferated, particularly in immuno-compromised patients such as those with AIDS. See GOODMAN AND GILMAN'S PHARMACOLOGICAL BASIS OF THERAPEUTICS, 1024-33 and Table 44-1 (8th ed., 1990), for additional microbial pathogens, diseases, and current therapeutic agents. The above-described cells and strains thereof are generally available, for example, from the American Type Culture Collection, Manassas, Va.

3.3.2 Resistant Mutation Mapping Method

Mutations providing resistance to a drug can usually be selected by growing the microorganisms in the presence of the candidate drug. Knowledge of which genes are able to mutate and provide resistance to the drug, provides information as to the mechanism of action of the drug. This method is especially useful in instances wherein the microorganism cannot gain resistance to a drug by plasmid (extrachromosomal) or transposon-based drug resistance elements, referred to as “extrinsic resistance.” Such extrinsic resistance occurs when microorganisms obtain these elements from other species or strains in the environment.

Thus, use of the preferred resistance mapping step of the target identification method is based on “intrinsic resistance,” wherein the resistance is conferred by intrachromosomal gene mutations. The frequency with which such intrinsic resistance mutations arise in a gene is helpful for predicting the utility of the compound in a clinical setting. Specifically, if the intrinsic resistance levels are too high, then further development of the compound may not be justified. Alternatively, if the intrinsic resistance levels are low, then development of a compound towards the gene is warranted. However, the rate of occurrence for extrinsic resistance to a compound or composition cannot be predicted.

Cells are selected which are resistant to each top priority compound in the focused library. These cells and the information gathered therefrom will be used in two ways. First, if intrinsic resistance is easily selected at a high rate, then the compound will be assigned a lower priority value. Second, mutations causing resistance are mapped and the identity of the genes conferring resistance determined. The mechanism of action of the compound is likely to involve the pathway in which the gene or genes conferring resistance functions. An obvious exception to this example would be with genes, which provide resistance by means of pumping the compound out of the cell (e.g., efflux pumps).

Mapping of the location of resistance mutations is accomplished, preferably, by (1) constructing genomic libraries from the resistant mutants, (2) identifying nucleic acids with aberrant sequences, (3) transferring sequences possessing the train of dominant resistance by means of transformation or transfection into a host which is sensitive to the drug, and (4) sequencing of the insert from cells which receive plasmids which confer such resistance. In the event that the inserted sequence contains multiple genes, each gene would then be separately tested to determine whether it is responsible for the resistance phenotype observed.

A first-step for resistant mutation mapping of the target of an anti-microbial compound is to obtain a bacterial strain that is resistant to concentrations of that compound that otherwise would kill the organism (Rice et al., GENETIC AND BIOCHEMICAL MECHANISMS OF BACTERIAL RESISTANCE TO ANTIMICROBIAL AGENTS (4^{th} ed., Williams and Wilkins, 1996); and Hooper et al., THE QUINOLONES: MODE OF ACTION AND BACTERIAL RESISTANCE (3d ed., Williams and Wilkins, Baltimore, Md. 1991). One method is to inoculate a susceptible strain on concentrations of the antibiotic far in excess of the MIC—this allows selection of single-step mutants (Rice et al., 1996 and Hooper et al., 1991). The rate of a single-step mutation is generally 10⁻⁸ to 10⁻⁷/CFU. If no mutants are obtained at this rate, then exposure to mutagenizing agents as described by Miller (Miller, J. H. 1972. EXPERIMENTS IN MOLECULAR GENETICS. Cold Spring Harbor Laboratory) such as nitrosoguanidine or UV muatagenesis may be done to increase the probability of obtaining a resistant mutant.

Once an antibiotic-resistant strain is obtained, the location of the mutation is determined. The preferred methods used to identify the mutation depend on the genetic tools available for that bacterial organism. In general, it is best to transfer the mutation from the resistant strain to a new strain especially if mutagenic agents were used to obtain the mutation. This allows isolation of the mutation responsible for the resistant phenotype. The transfer can be accomplished by P1 transduction in E. coli using the method of Silhavy (Silhavy et al., 1984. EXPERIMENTS WITH GENE FUSIONS. Cold Spring Harbor Laboratory, Cold Spring Harbor, N.Y.), or by any other transducing phage effective in other organisms such as phage 11 which is useful for S. aureus.

For E. coli a well known strategy to map the position of mutation is to begin in a set of Hfr strains which have different points of origin (Miller, J. H. 1972. EXPERIMENTS IN MOLECULAR GENETICS. Cold Spring Harbor Laboratory; and Singer et al., 1989, Microbiol. Rev. 53 (1): 1-24). This allows identification of the general region of the mutation. Then the mutation can be more-accurately mapped as described by Singer et al., 1989) through P1 transduction of a set of strains incorporating Tn10 transposons genetically linked to each other over the entire genome. This two-step method can narrow the mutation to within approximately 44 kb.

Next, the resistance locus region is cloned and sequenced to map the mutation more precisely. The locus region can be cloned from the mutant strain for transfer to a wild type strain to functionally determine that it is responsible for resistance. If a plasmid containing the resistance region confers resistance to a susceptible strain, then the mutation is a dominant resistance mutation. Deletion of regions of the insert DNA can serve to functionally identify the region which has the resistance mutation. That small piece of DNA can then be sequenced. If the transferred resistant strain locus does not confer resistance to a sensitive strain, then it is possible that the resistance mutation is recessive. An example of this is the rpsL mutation that confers streptomycin resistance. A wild-type streptomycin resistance locus, when cloned into a plasmid and introduced into a resistant strain, confers sensitivity. Deletions of regions of the wild-type DNA insert can be used to functionally identify the region of DNA, and thus the gene, that confers sensitivity to the antibiotic.

Beginning with a resistant strain, another method for identifying the target of an antibacterial compound is the shotgun cloning-based method reported by Caceres et al., 1997, J. Bacteriol. 179 16: 5046-55. In this procedure, a plasmid library can be constructed with randomly-sheared or enzymatically-digested fragments to genomic DNA from a strain resistant to a compound. The library can then be introduced into a susceptible strain. Selection for the plasmid is done followed by replica plating onto media containing concentrations of the compound above the MIC. Resistant colonies obtained may yield a plasmid DNA insert fragment containing the resistance mutation. Sequencing of the insert DNA and comparison with DNA from the susceptible strain identifies the mutation.

In some cases, resistance mutations may be located in the promoter region located upstream of the antibacterial target. The up-regulation of the gene product can give rise to a resistance phenotype (Chopra, 1998, J. Antimicrob. & Chemother. 41: 584-8). Thus, another way to identify targets of novel antibiotics can be through overexpression of the wild type gene product to confer resistance (Chopra, 1998). An example is in D-alanine racemase gene overexpression which was reported to confer resistance to D-cycloserine in Mycobacterium smegmatis (Caceres et al., 1997). Resistance, or susceptibility rescue through overexpression has been described in previous sections.

3.3.3 Drug Prioritization

Cells which are resistant to top priority compounds are selected. Top priority compounds are those which have been previously shown to be capable of entering a cell and thus capable of having antimicrobial effect. Focused libraries should be limited to only those compounds capable of potentially perturbing cell growth. The drugs can be further focused whether they are known to target genes which are conserved in an animal (e.g., human or agricultural animal) such that the drug may cause an unwanted side effect.

The process of drug compound prioritization according to the present inventive method comprises the following steps: Firstly, a chemical compound library consisting of discrete compounds derived from selected chemicals, natural products, herbal extracts, or produced by combinatorial chemistry is screened against whole cells of one or more bacterial or fungal species. Compounds which inhibit the growth or viability of the cells are identified. These compounds are potential leads for development of antimicrobials and antifungals because they are capable of entering cells of one or more microbial species and reducing the growth or viability of those cells. Secondly, the molecular target of active compounds is identified. Knowledge of the target of these compounds will increase their value, because subsequent assays can be better designed for optimization of the compounds and because the likelihood of specific toxicity can be predicted. For example, if the target is absent from the human host genome, or in the absence of a complete human genome for examination, if the target is missing from other mammalian genomes, or the C. elegans or D. melanogastergenome, then it is likely that compounds which inhibit that target will have no specific toxicity in humans. Knowledge of the cellular target provides an important prioritization of active compounds identified by the whole cell screen.

Prior to identification of the cellular targets, the compounds may be characterized further by additional tests against whole cells. For example, additional species may be included in the whole cell inhibition tests, such as strains which are resistant to known antimicrobials. These may be used to identify compounds which are effective against drug-resistant strains. Minimal inhibitory concentrations (MICs) of each compound or composition against the range of species may be determined by standard disk zone or agar dilution (see guidelines from the National Committee for Clinical Laboratory Standards (NCCLS), 940 West Valley Road, Suite 1400, Wayne, Pa. 19087-1898) assays, and compounds may be prioritized according to potency against one or more species. Compounds may also be tested against one or more mammalian cell type to determine if the compounds are likely to exhibit any toxicity toward human cells. Such potentially toxic compounds might be of lower priority.

The top priority compounds are then tested in order to identify the cellular target or target pathway. According to the present invention, the identity of the target for cell-inhibitory compounds is determined by applying up to three methods, collectively known as “genomics-assisted rapid identification of targets” (GARIT).

If intrinsic resistance is easily selected at a high rate (e.g., at a frequency greater than 10⁻⁶-10⁻⁷), then the compound will be awarded a lower priority. Mutations, which are not as easily selected, are awarded a higher priority.

Once mutants are identified they will be mapped to determine the identity of the genes which can confer resistance. The mechanisms of action of the drug is likely to involve a pathway in which the gene conferring resistance acts. An exception to this rule, would be genes which provide resistance by pumping the compound out of the cell.

Once the gene or genes are identified which confer resistance to the drug screened, then it will be awarded a weighted score for use in correlating the genes identified via this sub-method with the genes identified by the two other sub-methods used in target prediction.

3.4 Other Methods 125

In addition to the preferred steps of 3.1-3.3(110-120 of the GARIT technique 100) for the process of predicting a target gene or gene product and the drug(s) which targets said gene or gene product, additional submethods or processes can also be used for the prediction process 125. Preferably at least three submethods are used (with the methods of sections 3.1 to 3.3 being preferred), although two submethods can also be utilized for prediction. Additional submethods which can be used include three hybrid screening assay (Y3H), metabolic profiling, and proteomic profiling.

3.4.1 Three Hybrid Screening Assay

An alternative step 125 for use in the gene/drug prediction process is the use of a three hybrid screening assay. Use of a three hybrid screening assay is particularly useful for screening chemical libraries of small chemical compounds for agents that can bind targets in the system and the genes or gene products which bind the small molecules. Ideally, the small molecules are less than 1000 Daltons, preferably about 50 to about 800 Daltons. However, larger molecules which are the size of cyclosporin (1200 Daltons) or larger (about 1500 Daltons) can also be studied. The protocols and kits for the three hybrid screening assay are as described in U.S. Pat. No. 5,928,868 to Liu et al., and as described in Licitra et al., 1996 Proc. Natl. Acad. Sci. USA 93: 12817-21 (1996).

Briefly, the three hybrid assay involves the formation of a complex between a hybrid ligand, and two hybrid proteins in which one component of the three hybrid complex may be unknown. The unknown component in the assay may be either the small molecule contained in the hybrid ligand, or one of the hybrid proteins. There is no requirement that the unknown component be purified prior to the screening assay. Indeed, it is expected that the unknown component be in a mixture containing a large number of components (e.g., herbal extract), some or all of which can be unidentified. These interactions may be determined in vivo or in vitro. In the assay, a three hybrid complex triggers the expression of at least one reporter gene that can be detected by an appropriate technique.

For purposes of the gene identification and/or prediction process, the Y3H assay is suitable for: (1) determining the identity of target molecules having a binding affinity with a known small molecule (e.g., drug) where the small molecule has pharmacologic activity and where the target molecules may be suited for therapeutic intervention in a variety of disease states; (2) determining the identity of a small molecule capable of direct binding to a known target molecule where the identified small molecules may be suitable as therapeutic agents; (3) determining the identity of a small molecule capable of binding competitively to a known target molecule in the presence of a hybrid molecule so as to inhibit the binding between the target and the preselected small molecule; (4) developing a high throughput pharmacological assay in a number of cell types and organisms to screen for drug candidates; and (5) selecting novel small molecules for binding novel targets with high affinity using an iterative process of direct and competitive screening steps. For example, a known small molecule may be used to identify a target and subsequently the target may be used to identify a novel small molecule agents.

The three-hybrid protocol includes the step of providing a hybrid molecule, consisting of two covalently-linked small ligands identified as ligand A and ligand B. Ligand A has a specificity for a predetermined target and ligand B is the small test molecule. In contrast to ligand A, ligand B can be a random molecule of unknown identity obtained from a combinatorial library, or other compound archive. Examples of combinatorial libraries include but are not limited to peptide libraries, nucleic acid libraries, polysaccharide libraries, and small organic molecules. Archives of molecules include collections of environmental molecules and molecules from chemical processes.

The covalent hybrid linkage between ligand A and ligand B may be formed by any of the methods known in the art. For example, see J. March, ADVANCED ORGANIC CHEMISTRY (John Wiley & Sons Inc 1985); and House, MODERN SYNTHETIC REACTIONS (Benjamin Cummings 1972). Descriptions of linkage chemistries are further provided by Crabtree et al. (WO 94/18317 and WO 95/02684), Schreiber et al. (WO 96/13613), and Holt et al. (W096/06097).

According to the method of the invention,the hybrid ligand is introduced into a sample, the sample containing an environment. The environment is characterized by a functional transcription and translation apparatus. This environment may be whole cells, cell lysates or a synthetic mixture of enzymes and reagents. It is desirable that components of the assay including vectors and hybrid molecules be readily introduced into the environment. An example of an environment that is cellular, is eukaryotic cells, more particularly a yeast cell population (e.g., Saccharomyces cerevisiae or Schizosaccharomyces pombe). Other examples include the cells and microorganisms listed herein.

Cells which may be used in a three hybrid assay include primary cultures, cultures of immortalized cells or genetically manipulated strains of cells. Preferred cell types are those with increased permeability to the selected hybrid ligands. Another criteria for selection of a particular cell type may be the desirability of post-translational modification of proteins. The binding of such modified proteins to a small molecule may more accurately mimic the natural state of the cell. The assay may be performed using single cells or populations of cells for each test sample.

The introduction of the hybrid ligand into the environment, may include traversing a membrane so as to enter the cell. The hybrid molecule is introduced into cells by electroporation or any permeation procedures known in the art. In certain embodiments, cells may be used which may be genetically or pharmacologically modified to increase the intracellular concentrations of the hybrid ligand. These include procedures that utilize polybasic peptides (e.g., polymixin B) or genetically altered strains of cells which offer increased permeability or decreased efflux of hybrid ligand. A hybrid ligand may be selectively formed having an overall charge and polarity that facilitates transmembrane transport.

In the three hybrid assay, the environment contains three different types of vector. Two of the vectors encode fusion or hybrid proteins; each hybrid protein including a transcription module and a target molecule for binding ligand A or ligand B of the hybrid ligand. When a three hybrid complex is formed, the transcription modules are brought into close proximity, and the transcriptional activation of a reporter gene occurs. Methods of preparing the necessary vectors, in light of the disclosure in U.S. Pat. No. 5,928,868 are well known to one skilled in the art. Additional methods are described in Sambrook et al., MOLECULAR CLONING: A LABORATORY MANUAL, vol. 1-3 (Cold Spring Harbor Laboratory Press, 1989); Ausebel, CURRENT PROTOCOLS IN MOLECULAR BIOLOGY (John Wiley & Sons, 1995); Silhavy et al., EXPERIMENTS WITH GENE FUSIONS (Cold Spring Harbor Laboratory Press 1984); Gerhardt et al., METHODS FOR GENERAL AND MOLECULAR MICROBIOLOGY (American Society for Microbiology, 1994); Lorian, ANTIBIOTICS IN LABORATORY MEDICINE, 4^{th} ed., (Williams & Wilkins, 1996); and Murray et al., MANUAL OF CLINICAL MICROBIOLOGY, 7^{th} ed. (American Society for Microbiology, 1999).

One utility provided by this step in the gene or gene product prediction process is identification of the target of a small molecule with known pharmacological function. The target molecule may be any cellular component including a nucleic acid, a polysaccharide, a lipid or a protein or a combination of any of these. For instance, when the target is a protein encoded by DNA, a cloned DNA encoding target protein may be inserted by standard cloning techniques. Alternatively, random DNA sequences of a size that is capable of encoding a yet undetermined target protein, may be inserted in the second expression vector where the random DNA sequences are derived from a genomic DNA library, a cDNA library or a synthetically generated library formed from eukaryotic cells, prokaryotic cells, viruses or formed by an automated DNA synthesizer. Examples of target proteins encoded by a plasmid library may include enzymes, oncogene products, signaling proteins, transcription factors soluble domains of membrane proteins or any essential protein described herein. An alternative application of the three hybrid assay is when the nature of the target molecule is known and a small molecule is sought that is capable of binding the target molecule. This type of assay may be performed as a direct assay or a competitive binding assay.

The third vector contained in the environment is a vector encoding a reporter protein. The reporter is switched on in the presence of united transcription activation modules. Reporter genes are so named because when transcribed and translated, they can be detected according to a phenotype based on a selectable characteristic such as growth in an appropriate growth media or visual screening. In a preferred embodiment of the invention, reporter genes that permit visual screening are utilized. Examples of reporter gene products that may be detected visually include β-galactosidase, Aequorea victoria Green Fluorescent Protein (GFP) and Blue Fluorescent Protein (BFP), luciferase, antibodies or selected antigens. These gene products may be identified visually or by spectrophotometric quantification.

The switching on or off of the reporter gene depends in part on the affinity of the small molecule ligand for the target. The affinity of a ligand or small molecule for a target molecule may vary substantially in the three-hybrid screen. An example of the ranges of binding affinities includes a K_{d} having a value below 10⁻⁶, more preferably below 10⁻⁷ and even more preferably below 10⁻⁸ and in some embodiments below about 10⁻⁹. An example of a dissociation constant includes a range of preferably less than or equal to 10 μM. This does not preclude the effectiveness of a binding affinity outside this range.

A feature of the three-hybrid system includes the formation of a hybrid ligand molecule. The binding of the hybrid molecule to both target hybrid molecules produces a three hybrid complex that results in the stimulation of transcription of at least one reporter gene. A detectable result may follow from direct binding of a hybrid ligand to target hybrid molecules or by competitive binding of the hybrid ligand acting as an agonist or antagonist. In certain circumstances, the target molecule for therapeutic intervention may be known but a suitable small molecule for binding the target molecule may be desired. If no candidate small molecule for binding the target is known, it may be desirable to generate a random library of hybrid molecules in which a mixture of small molecules are chemically modified to bind to a preselected ligand. Subsequently, pools of molecular hybrids may be introduced into an environment such as yeast cells for performing the three-hybrid protocol. Those samples that are positive can be re-analyzed using iteratively smaller subsets of the initial pool until a single candidate small molecule type is identified.

In another embodiment, a molecule that binds a selected target may be known, but identification of componds related (e.g., homologs, derivatives,mimetics or analogs of the compound) to the known molecule which have binding affinity than that of the target may be preferred. In this situation, a hybrid ligand of the known molecule and a ligand is formed and the three hybrid screening assay is performed in the presence of a library of compounds that compete with the hybrid ligand for binding the target. Samples which contain molecules having greater binding to the target molecule, relative to the known compound, will not activate the reporter gene.

3.4.2 Metabolic Profile

Another method 125 which can be used in the molecular target identification is metabolic profiling.

Metabolic profiling can be performed, for example, by analyzing the levels of metabolites within cells treated with various doses of a compound, e.g., an antimicrobial agent. In principle, methods such as mass spectrometry may be applied to whole cell lysate supernatants to measure changes in the amounts of a multitude of cellular metabolites in response to drug treatment. More commonly, effects are measured on the macromolecular biosynthetic capabilities of cells. See for example, Broetz et al., 1995, Antimicrob. Agents & Chemotherap. 39: 714-19, which discloses the biosynthesis of DNA, RNA, protein and cell walls in cells treated with the drug, mersacidin, and demonstrated that this antibiotic inhibits cell wall biosynthesis. Similarly, Adrian et al., (2000, Antimicrob. Agents & Chemotherap. 44: 732-8) measured DNA, RNA, protein, and cell wall biosynthesis in cells treated with the drug, everninomycin, and concluded that this antibiotic inhibits protein synthesis. Experimentally, for example, cells may be incubated with the antibiotic and radiolabeled thymidine, UTP, N-acetyl glucosamine, acetate, or isoleucine can be used to measure rates of synthesis of DNA, RNA, cell wall, fatty acid or protein biosynthesis, respectively, according to methods known in the art.

3.4.3 Proteomic Profile

Proteomics is another alternate step 125 of the GARIT process which can be used in the prediction process of identifying molecular targets. Proteomics, as discussed below, is more an array of tools that have been developed to assess protein modulation at the level of protein expression, and not at the level of mRNA expression. For a summary of proteomic techniques, see Borman, 2000 Chem. & Engin. News 78: 31-37.

Proteomic techniques analyze the protein ensemble of a cell, encoded by the genome and serve as the emerging key between genomics and pharmacogenomics. This technology studies the proteins encoded by the genes of a cell of interest and includes determining protein expression (identifying and quantifying the proteins expressed), characterizing the protein (e.g., assessing post-transitional modification), transcription profiling (e.g., determining which genes are transcribed into RNA in a particular cell type, developmental state or disease state), high-throughput expression and purification of proteins, determining protein function, identifying protein interactions, and exploring the correlation of proteins with disease. Like genomic studies using microarrays, proteomics seeks to determine the cellular composition of proteins at any particular time. Unlike DNA microarrays, proteomics provides information on (1) when predicted gene products are translated, (2) relative concentrations of gene products, and (3) the extent of post translational modification. None of these can be accurately predicted from the nucleic acid sequence alone.

Proteomic analysis encompasses techniques such as multiplex high-throughput screens using two hybrid analysis, fluorescence resonance energy transfer (FRET), extrapolation of the proteins expression based on RNA arrays, proteomic interaction chip arrays, two dimensional gel electrophoresis which can characterize proteome profiles of up to about 10,000 proteins, and high resolution mass spectrometry (HRMS). Additional methods include transcriptional profiling, high-throughput expression and purification of proteins, pathway analysis to study signal transduction and other complex cell processes, large-scale protein folding and 3-dimensional structure studies, and bioinformatics analysis of proteomics data. Preferred methods of performing proteomic analysis are two-dimensional gel electrophoresis and high-resolution mass spectrometry(HRMS) and can be performed as described by Washburn et al., 2000 Curr. Opin. Microbiol. 3: 292-7. An alternative method comprises the use of isotope-coded affinity tag (ICAT) reagents with tandem mass spectrometry (MS/MS) as described for example by Ideker et al., 2001 Science 292:929-934.

Methods of obtaining proteomic information include the yeast two hybrid system or the yeast three hybrid system. The yeast two hybrid system, which uses a bait protein-prey protein combination to induce transcription if the bait and prey proteins bind, has recently been used to create the first protein-protein interaction map of an entire organism, yeast (S. cerebisiae). See Uetz et al., 2000 Nature 403(6770): 623-7. This is a useful way of determining protein-protein interactions. A preferred method uses the yeast three hybrid system, as described in U.S. Pat. No. 5,928,868.

Another proteomic method is two-dimensional (2-D) electrophoretic gel analysis. Typically, electrophoretic gels separate proteins in one dimension based on size and a second dimension based on charge. Software has been developed which facilitates 2-D gel analysis, such as Melanie (Geneva Bioinformatics, Geneva Switzerland and BioRad Laboratories, Hercules, Calif.), PDQuest (BioRad), ImageMaster (Amersham Pharmacia Biotech AB, Sweden), Phoretix 2D (Phoretix International, England), Gellab (Scanalytics, Fairfax, Va.), and Kepler (Large Scale Proteomics, Rockville, Md.). Such software can be used to further analyze the results obtained by 2D gels to determine which protein has been modulated by exposure to a particular agent. The information obtained by such gels can then be stored in databases for future reference and analysis.

Bioinformatics can be employed as part of the proteomic step, when biological information on various proteins, as perhaps acquired by 2D gel analysis or other methods, are indexed in a manner which permits annotating and interpreting experimental results from proteomic studies or other studies discussed herein.

Proteomic analysis of microorganisms can be performed as described for Mycoplasma genitalium (Wasinger et al., Eur. J. Biochem. 267: 1571-82, 2000), Spiroplasma melliferum (A56) (Cordwellet al., Electrophoresis 18(8):1335-46, 1997), and Haemophilus influenzae type-strain NCTC 8143 (Link et al., Electrophoresis 18(8): 1314-34, 1997). Rapid protein display profiling of cancer progression directly from human tissue using a protein biochip can be performed, for example as described by Paweletz et al., 2000 Drug Devel. Res. 49: 34-42 and Washburn and Yates, 2000 Curr. Opin. Microbiol. 3: 292-7. Additional methods of performing proteomic analysis can be performed, for example, as described by Michael J. Dunn, FROM GENOME TO PROTEOME: ADVANCES IN THE PRACTICE AND APPLICATION OF PROTEOMICS (Verlagsgesellschaft, 2000); Suhai, GENOMICS AND PROTEOMICS: FUNCTIONAL AND COMPUTATIONAL ASPECTS (Plenum Publishing,2000);Pennington et al., PROTEOMICS: FROM PROTEIN SEQUENCE TO FUNCTION (Springer Verlag, 2000); Humphery-Smith et al., J. Protein Chem. 16(5): 537-44 (1997); and P. Jolles et al., PROTEOMICS IN FUNCTIONAL GENOMICS: PROTEIN STRUCTURE AND ANALYSIS (Birkhauser, 2000).

Proteomic data may be analyzed for microorganisms and other cells in a manner similar to that for microarrays. Proteomes of wild-type cells to which no composition or compound was administered can be compared with proteomes of the same microorganism or cell to which a composition or compound has been administered. The differences in protein expression profiles would then be assessed to identify which proteins are targeted by the composition or compound. The information thus obtained would then be compared to the genes or gene products identified using preferably two of the other methods described herein. Preferably, proteomic methods would supplement the preferred methods of gene prediction discussed above.

3.5 Analysis of the Data 130

Once data is obtained from at least two, and preferably at least three, and more preferably from at least the methods set forth in Sections 3.1-3.3 (110-120), the data will be analyzed 130 to predict a gene or genes which are targeted by the drug or composition screened.

In one embodiment,if the methods of sections3.1, 3.2 and 3.3 are utilized and each identifies a common gene, then this common gene will be subjected to a validation step. If more than one common gene is identified by all three methods, then, all the genes thus identified will be subjected to analysis validation, unless the genes thus identified are known to encode drug modification enzymes or efflux pumps.

In another embodiment, if a common gene is identified by only two of three prediction methods described above, then the gene identified by only two of three methods will be subjected to validation analysis.

The gene expression profiles developed are further contemplated for use as part of a computerized database or system. This computerized database can then be used for comparisons of expression profiles prepared from cells which have been treated with new compounds. Another embodiment contemplated includes a database of gene expression profiles resulting from down-regulation of each of the top priority conserved, targets. As information is added to this database, the validation step utilizing the microarrays would be accelerated, as a database of previously performed profiles would exist for each top priority target.

Weighting the data to predict the gene(s) 130 identified by the prediction portion of process (105 to 130) would be performed as follows. Genes identified by the methods of 110 to 125 would be weighted according to the method (110, 115, 120 and 125) used. From highest weight (more likely to be the predicted gene) to lowest weight (less likely to be the predicted gene), the methods are weighted as follows: mapping resistance mutations (120)>transformation selection (110)>yeast three-hybrid (125)>microarray-based gene expression profile (115)>metabolic profile (125)>proteomic profile (125).

This weighting can similarly be used in the validation step 150.

4.0 Validation of Target

Once a target is predicted as described using the methods of Section 3 (105-130), then it must be confirmed by using one or both of the validation steps described below (135-150). Validation is required because with the complex set of interactions between genes, the proteins they encode and the pathways and feed back loops that they may be part of, a predicted target may be false.

Mutations to resistance may reside in genes which encode efflux pumps or drug-modification enzymes or in the promoters of such genes. These genes and promoters are not actually the molecular targets of a drug's antimicrobial activity, but the products of the genes act on the drug to reduce its effective concentration, thus giving the indication that the organism or cell carrying such a gene is “resistant.” Similarly, the expression profile determined by use of microarrays exposed to sublethal doses of compounds may reveal up-regulation of efflux pumps. Even when using transformation selection, overexpression of efflux pump genes can rescue the organisms from the lethality of the compound. Thus, a validation step is necessary to characterize the target of the compound or composition.

The first method of validation includes preparation of a microorganism or cell which underexpresses the predicted target, thus rendering the microorganism 140-145 or cell hypersensitive or hypersusceptible to the compound or agent 105. The second method includes the preparation of a nucleic acid microarray corresponding to a microorganism in which the predicted gene target has been placed under a regulatable promoter. The native form of the predicted gene target has been functionally knocked out 155. The microorganism is then manipulated to underexpress the predicted gene and the resulting microarray determined gene expression profile acquired from these cells (Profile B) 155 is compared to a microarray-determined expression profile of the wild-type microorganism exposed to the drug (Profile A) 115. If the microarray-generated profiles are similar, then the predicted gene and the drug which it targets have been validated. This process can be performed using the methods described by Marton et al., 1998, Nature Med. 4: 1293-1301.

4.1 Underexpression Assay 140-145

In the underexpression assay 140-145 of a GARIT method 100, the gene product(s) predicted using the processes of Section 3.0 is (are) further analyzed in the microorganism or cell of interest as follows. A construct placing a wild-type copy of the predicted gene under the regulation of a preferred promoter (e.g., P_{BAD , P}_{lac/ara-1} and P_{tetO-1} or other suitable promoters discussed herein) is incorporated into the microorganism, preferably into the genome. Alternative promoters which can be utilized are described in WO 99/52926. The native copy of the predicted gene target or gene encoding a targeted gene product is then functionally deleted such that it is no longer expressed by the microorganism or cell. The recombinant microorganism or cell expressing a regulatable form of the predicted gene is exposed to the drug believed to target the predicted gene, as the gene is regulated to be underexpressed by the recombinant microorganism. If the recombinant microorganism is hypersensitive to the drug when the predicted target gene is under expressed,then the association between the predicted target gene and the drug has been validated using this assay. The methods utilized to validate the gene target by this underexpression assay are those described above in Section 3.1 and in Examples 1 and 2.

4.2 Gene Expression Profile 155

For the step of validation using a microarray 155, a microorganism in which the targeted gene has been functionally removed (knocked out) must be prepared. In this step, the microorganism will first have a copy of the wild-type target gene under control of a regulatable promoter inserted into the microorganism, as discussed in Section 3.1 above. Additionally, the native copy of the gene is functionally knocked out such that it can no longer produce a functional copy of the protein encoded by it. A microarray hybridization experiment is then performed on total RNA from the recombinant microorganism which has been regulated to underexpress the predicted target gene and comparing the gene expression levels to those from unengineered, normal cells. The microarray experiment is performed in the manner described in section 3.2

The gene expression profile of the underexpressed recombinant cell (Profile B) 155 is then compared to the gene expression profile prepared in the gene prediction step (Profile A) 115 of the target identification process. If the Profile B 155 is similar (e.g., the same genes are activated and inactivated with a statistical significance of greater than 95%, and preferably greater than 98%) to the Profile A 115; then the target of the composition and/or compound and the gene or gene product targeted has been validated. Statistical processing of the data to subtract backgrounds and normalize the results to each experiment and between experiments can be performed using such software as GeneSpring (Silicon Genetics, San Carlos, Calif.).

4.3 Analysis of Data 150

In the event that the prediction and validation methods described above do not identify common genes, then each of the genes identified by at least one of the validation methods can be further analyzed individually. In another embodiment, validation can be performed using only one of the two validation steps, although preferably both steps of the validation process (140-145 and 155) are performed for validating the gene or genes and the drug or drugs which target said genes. Weighting of the data can be performed as described above.

5.0 Agents to be Screened

A variety of agents are contemplated for screening using the processes of gene and drug identification described herein. Preferred agents are those which regulate a pathogen and include antiamebic, antibacterial, antifungal, antimalarial, antiprotozoal and antirickettsial agents. Alternative agents include those which regulate genes in multicellular eukaryotes, such as humans and preferably cancer cells.

Antiamebic agents contemplated for screening include, but are not limited to: arsthinol, bialamicol, carbarsone, cephaeline, chlorbetamide, chloroquine, chlorphenoxamide, chlortetracycline, dehydroemetine, dibromopropamidine, diloxanide, diphetarsone, emetine, fumagillin, glaucarubin, glycobiarsol 8-hydroxy-7-iodo-5-quinoline-sulfonic acid, iodochlorhydroxyquin, iodoquinol, iaromomycin, phanquinone, polybenzarsol, propamidine, quinfamide, secnidazole, sulfarside, teclozan, tetracycline, thiocarbamizine, thiocarbarsone, tinidazole and all homologs and derivatives thereof.

Antibacterial agents include antibiotics and synthetic agents. Antibiotics contemplated for screening include, but are not limited to: aminoglycosides, amphenicols, ansamycins, β-lactams, lincosamides, macrolides, polypeptides, tetracyclines, cycloserine, mupirocin, and tuberin. Synthetic antibacterial agents include 2,4-diaminopyrimidines, nitrofurans, quinolones and analogs thereof, sulfonamides, sulfones, clofotol, hexedine, methenamine and analogs thereof, nitroxoline, taurolidine, xibornol and all homologs and derivatives thereof. Antibacterials also include leprostatic agents (e.g., acedapsone, acetosulfone sodium, clofazimine, dapsone, diathymosulfone, glucosulfone sodium, hydnocarpic acid, solasulfone, succisulfone and sulfoxone sodium), antirickettsial agents, tuberculostatic agents (e.g., p-aminosalicylic acid, benzoylpas, ethionamide, furonazine, etc.) and their homologs and derivatives.

Antifungal compounds contemplated for screening include, but are not limited to, polyenes and other compounds (e.g., azaserine, griseofulvin, oligomycins, neomycin undecylenate, pyrrolnitrin, siccanin, tubercidin, and viridin), as well as synthetic antifungals such as allylamines, imidazoles, thiocarbamates and triazoles and all homologs and derivatives thereof.

Antiprotozoal agents contemplated for screening include, but are not limited to, antiamebic agents as well as agents against Giardia, histomonas, Leishmania, malaria (antimalarial), pneumocystis, trichomonas, and trypansoma.

The agents to be screened also include the protein mimetics. “Mimetics” are molecules wherein the structure is determined from the knowledge of the structure of the protein encoded by the essential gene or portions thereof. Also contemplated for screening are homologs of any the above-identified agents as well as pharmaceutically acceptable salts of all the compounds, homologs and derivatives.

The invention also contemplates screening agents from chemical libraries, small molecule libraries, peptide libraries, and natural product extracts including for example extracts from Actinomycetes, fungi and plants.

6.0 Diseases Treated

Agents identified using the methods described herein can then be used to treat a variety of diseases caused by viruses, amebas, protozoa and bacteria. The agents can be used alone or in combination with other agents or with adjuncts, such as antibacterial adjuncts or potentiators (e.g., β-lactamase inhibitors, renal dipeptidase inhibitors and renal protectants).

Diseases and conditions induced by pathogens contemplated for treatment by identified agents include, but are not limited to: sepsis and septic shock, sinusitis, acute otitis media, serous otitis media, mastoiditis, external otitis (e.g., necrotizing otitis externa and perichondritis), laryngeal infections (e.g., acute epiglottitis, croup and tuberculous laryngitis), endocarditis, intraperitoneal abscesses, peritonitis, acute infectious diarrheal diseases (those caused by, for example: Vibrio cholerae, Clostridium perfringens, Staphylococcus aureus, Aeromonas hydrophila, Plesiomonas shigelloides, Giardia lamblia, Cryptosporidium, Shigella sp., Salmonella enteritidis, Campylobacter jejuni, enterohemorrhagic E. coli, enteroinvasive E. coli, Vibrio parahemolyticus, Clostridium difficile, Entamoeba histolytica, Salmonella typhi and Yersinia enterocolitica), bacterial food poisoning (by organisms such as Staphylococcus aureus, Bacillus cereus, Clostridium perfringens, Vibrio cholerae, Escherichia coli, Salmonella sp., Shigella sp. and Vibrio parahemolyticus), sexually transmitted diseases and related conditions (e.g., Chlamydia trachomatis, Treponema pallidum, Calymmatobacteriuum granulomatis, Ureaplasma urealyticum, Mycoplasma hominis, Gardnerella vaginalis and N. gonorrhoeae), urinary tract infections (by E. coli and Proteus), pyelonephritis, infectious arthritis (e.g., gonococcal induced, fungal induced, tuberculous induced and spirochetal induced), osteomyelitis and infections of prosthetic joints, skin and soft tissue infections (by Staphylococcus aureus, Pseudomonas aeruginosa, Mycobaterium ulcerans, M. leprae, M. tuberculosis, Streptococcus pyrogens), nocardiosis and actinomycosis.

As the preferred agents are those that inhibit bacteria, treatment of the following gram-positive bacterial infections are contemplated, but are not limited to: pneumococcal infections, staphylococcal infections, streptococcal infections, diphtheria, corynebacterial infections, Listeria monocytogenes infections and clostridial infections. Gram-negative infections contemplated for treatment with said agents include: meningococcal infections, gonococcal infections, Moraxella, Catarrhalis and Kingella infections, Haemophilus influenzae infections, Escherichia coli infections, Helicobacter pylori infections, Capnocytophaga infections, H. ducreyi infections, Legionella infections, pertussis, enteric bacilli infections, Pseudomonas infections, salmonellosis, shigellosis, Campylobacter infections, cholera and other vibrioses, brucellosis, tularemia, Yersinia infections, baronellosis and bacillary angiomatosis and donovanosis.

Treatment with said agents is also contemplated for mycobacterial diseases (e.g., tuberculosis, leprosy and Mycobacterium avium), spirochetal diseases (e.g., syphilis, endemic treponematoses, leptospirosis, Lyme borreliosis and relapsing fever), and rickettsial diseases (e.g., chlamydial infections and mycoplasma infections).

7.0 Compositions and Administration

The invention also relates to compositions which regulate the target gene or gene product. Such compositions are preferably antagonists or inhibitors of the essential gene, but can also include agonists, or agents which up-regulate gene activity. The compounds identified may be employed in combination with a non-sterile or sterile carriers for use with cells, tissues or organisms, such as a pharmaceutical carrier suitable for administration to a subject. Such compositions comprise, for instance, a media additive or a therapeutically effective amount of an agent identified by the assay described herein and a pharmaceutically acceptable carrier or excipient. Such carriers may include, but are not limited to, saline, buffered saline, dextrose, water, glycerol, ethanol and combinations thereof. The formulation should suit the mode of administration.

Identified agents which regulate the essential gene may be employed alone or in conjunction with other compounds, such as therapeutic compounds, in the form of a pharmaceutical composition.

The pharmaceutical compositions may be administered in any effective, convenient manner including, for instance, administration by topical, oral, anal, vaginal, intravenous, intraperitoneal, intramuscular, subcutaneous, intranasal or intradermal routes among others.

In therapy or as a prophylactic, the active agent may be administered to an individual as an injectable composition, for example as a sterile aqueous dispersion, preferably isotonic. Alternatively the composition may be formulated for topical application for example in the form of ointments, creams, lotions, eye ointments, eye drops, ear drops, mouthwash, impregnated dressings and sutures and aerosols, and may contain appropriate conventional additives, including, for example, preservatives, solvents to assist drug penetration, and emollients in ointments and creams. Such topical formulations may also contain compatible conventional carriers, for example cream or ointment bases, and ethanol or oleyl alcohol for lotions. Such carriers may constitute from about 1% to about 98% by weight of the formulation; more usually they will constitute up to about 80% by weight of the formulation.

For administration to animals, preferredly mammals, and particularly humans, it is expected that the therapeutically effective daily dosage level of the active agent will be from 0.01 mg/kg to 10 mg/kg, typically around 1 mg/kg. The physician, in any event, will determine the actual dosage which will be most suitable for an individual and will vary the amount based on factors such as patient age, weight and dose response. The above dosages are exemplary of the average case. There can, of course, be individual instances where higher or lower dosage ranges are merited, and such are within the scope of this invention.

Indwelling devices include surgical implants, prosthetic devices and catheters, i.e., devices that are introduced to the body of an individual and remain in position for an extended time. Such devices include, for example, artificial joints, heart valves, pacemakers, vascular grafts, vascular catheters, cerebrospinal fluid shunts, urinary catheters, continuous ambulatory peritoneal dialysis (CAPD) catheters. Agents identified by the method of the invention can be coated on, imbedded in, or otherwise combined with an indwelling device to prophylactically prevent infections.

Alternatively, the composition of the invention may be administered by injection to achieve a systemic effect against relevant bacteria shortly before insertion of an in-dwelling device. Treatment may be continued after surgery during the in-body time of the device. In addition, the composition could also be used to broaden perioperative cover for any surgical technique to prevent bacterial wound infections.

In addition to the therapy described above, the compositions of this invention may be used generally as a wound treatment agent to prevent adhesion of bacteria to matrix proteins exposed in wound tissue and for prophylactic use in dental treatment as an alternative to, or in conjunction with, antibiotic prophylaxis.

Alternatively, the composition of the invention may be used to bathe an indwelling device immediately before insertion. The active agent will preferably be present at a concentration of 1 μg/ml to 10 mg/ml for bathing of wounds or indwelling devices.

### EXAMPLES

### Example 1

### Cell Assay Using Under-expression of murA or folA in E. coli

The wild-type gene murA, which encodes the enzyme UDP-N-acetylglucosamine enolpyruvate transferase, was knocked out in E. coli, and the gene was placed under ectopic expression in the yibD locus under regulation by P_{BAD} with the regulator, L-arabinose.

UDP-N-acetylglucosamine enolpyruvate transferase,alanine racemase and D-alanine: D-alanine ligase are enzymes involved in the peptidoglycan biosynthesis pathway. UDP-N-acetylglucosamineenolpyruvate transferase catalyzes the fourth step, while alanine racemase, is at the tenth step immediately followed by D-alanine:D-alanine ligase. UDP-N-acetylglucosamineenolpyruvate transferase has been shown to be the target of phosphoenolpyruvate analogs, such as the antibiotic, phosphomycin (Samland et al., 1999, Biochemistry, 38: 13162-9; Horii et al., 1999, Antimicrob. Agents Chemother., 43: 789-93), whereas D-alanine analogs, such as D-cycloserine, inhibit both alanine racemase and D-alanine: D-alanine ligase (Caceres et al., 1987, J. Bacteriol. 179: 5046-55; Vicario et al., 1987, J. Antibiot. (Tokyo), 40: 209-16). Thus, the viability of cells under-expressing mura in the presence of phosphomycin or D-cycloserine is used to measure whether underexpression of mura gives rise not only to compounds that specifically inhibit its gene product, but also hypersensitivity to any compounds that inhibit the peptidoglycan biosynthesis pathway.

In another example, the wild-type native folA gene was knocked out, and a wild-type copy of the gene folA, which encodes the protein dihydrofolate reductase, was placed under ectopic expression in the yibD locus under regulation by P_{BAD} with the regulator, L-arabinose. Dihydropteroate synthetase, the gene product of sulA, catalyzes the enzymatic reaction two steps before dihydrofolate reductase in the folate biosynthetic pathway. SulA activity is inhibited by sulfone and sulfanilamide sulfa drugs, such as sulfamethozasone (De Benedetti et al., 1987, J. Med. Chem., 30: 459-64). The choice of folA allows verification that under-expression of a gene product will result in hypersensitivity both to compounds that directly inhibit it and to compounds that inhibit other steps in the enzymatic pathway to which it belongs.

Materials. The bacterial strains used are listed in Table 3. The E. coli plasmids utilized are listed in Table 4. The media and chemicals utilized in the experiments are as follows. Mueller Hinton broth (Difco; Cat. No. 0757-17-6), trimethoprim lactate (Sigma; Cat. No. T-0667), phosphomycin (Sigma; Cat. No. P-5396), chloramphenicol (Sigma; Cat. No. C-0378), ampicillin (Sigma; Cat. No. A-9518), kanamycin (Sigma; Cat. No. K-4000), tetracycline (Sigma; Cat. No. T-3383), sulfamethozasone (Sigma; Cat. No. S-7507), D-cycloserine (Sigma; Cat. No. C-6880), L (+) Arabinose (Sigma; Cat. No. A-3256), isopropyl β-D-thiogalacto-pyranoside (IPTG; Sigma Cat. No. 1-6758), anhydrotetracycline(Clontech; Cat. No. 8633-1), and dimethyl sulfoxide (Sigma; Cat. No. D-8418).

Methods. E. coli strains were routinely grown in LB media or on LB agar plates containing 1.5% agar (Sambrook et al., MOLECULAR CLONING: A LABORATORY MANUAL (2nd ed., Cold Spring Harbor Laboratory Press, 1989). MIC determination was performed by microbroth dilution-cultures grown in Mueller Hinton broth in 96-well microtiterplates purchased from Nunc (Plates—Cat. No. 262162; Lids—Cat. No. 264122). When required, media were supplemented with 35 μg/ml chloramphenicol, 50 μg/ml kanamycin, or 100 μg/ml ampicillin L (+) Arabinose was added to culture media at a final concentration of 0.2% for routine culture growth of E. coli strains GTC-EC#292 and GTC-EC#293. IPTG was added to a final concentration of 0.1 mM and anhydrous-tetracycline was added to a final concentration of 100 ng/ml to the culture media for routine growth of GTC-EC#313 and GTC-EC#346, respectively.

TABLE 3
Bacterial strains

| Strain | species | genotype | source | Reference/cat # |
|---|---|---|---|---|
| MC1061 | E. coli | F⁻araD139 Δ(ara-leu)7697 galE15 galK16 | | Wertman et al., 1986, |
| | | (lac)X74 rpsL (Str^{R}) hsdR2 (r_{K}⁻m_{K}⁺) mcrA mcrB1 | | Gene 49: 253-62 |
| KM354 | E. coli | argE3 his-4 leuB6 proA2 thr-1 ara-14 galK2 | Keenan Murphy | Murphy, 1998, J. |
| | | lacY1 mtl-1 xyl-5 thi-1 rpsL31 tsx-33 supE44 recJ | | Bacteriol. 180: 2063- |
| | | | | 71 |
| KM29 | E. coli | argE3 his-4 leuB6 proA2 thr-1 ara-14 galK2 | Keenan Murphy | Murphy, 1998 |
| | | lacY1 mtl-1 xyl-5 thi-1 rpsL31 tsx-33 supE44 | | |
| | | Δ(recC ptr recB recD)::P_{lac}-bet exo kan^{R} recJ | | |
| DH5αPRO | E. coli | f⁻l⁻ deoR endA1 gyrA96 hsdR17(r_{K}⁻m_{K}⁺) recA1 | Clontech; Cat. No. | N/A |
| | | relA1 supE44 thi-1 (lacZYA-argFV169) f80dlacZ | 1624-1 | |
| | | ΔM15 attB::(P_{N25}-tetR P_{lacI}^{Q}-lacI Sp^{R}) | | |
| GTC-EC#292 | E. coli | MC1061 (pTP223, pBL21PRO) yibD::(P_{BAD}- | Deposit no. | N/A |
| | | murA Ap^{R}) ΔmurA::Km^{R} | PTA-1935_{—} | |
| GTC-EC#293 | E. coli | MC1061 (pTP223, pBL21PRO) yibD::(P_{BAD}-folA | Deposit no. | N/A |
| | | Ap^{R}) ΔfolA::Km^{R} | PTA-1933 | |
| GTC-EC#313 | E. coli | MC1061 (pTP223, pBL21PRO) yibD::(P_{lac/ara-1}- | Deposit no222. | N/A |
| | | folA Ap^{R}) ΔfolA::Km^{R} | PTA-1930 | |
| GTC-EC#346 | E. coli | MC1061 (pTPKAN) attB::(P_{N25}-tetR P_{lacI}^{Q}-lacI | Deposit no. | N/A |
| | | Sp^{R}) yibD::(P_{LtetO-1}-folA Ap^{R}) AfolA::Km^{R} | PTA-1934 | |
| GTC-EC#302 | E. coli | DH10B (pBC.SK yibD::(arabinose p_{BAD}-folA)) | Deposit no. | N/A |
| | | | PTA-1931 | |
| GTC-EC#318 | E. coli | MC1061 (pBC.SK yibD::(arabinose P_{lac/ara-1} MCS | Deposit no. | N/A |
| | | Ap^{R})) attB::(P_{N25}-tetR P_{lacI}^{Q}-lacI Sp^{R}) | PTA-1932 | |
| GTC-EC#321 | E. coli | MC1061 (pBC.SK yibD::(arabinose P_{LtetO-1} MCS | Deposit no. | N/A |
| | | Ap^{R})) attB::(P_{N25}-tetR P_{lacI}^{Q}-lacI Sp^{R}) | PTA-1929 | |
| DH10B | E. coli | f⁻mcrA Δ(mrr-hsdMRS-mcrBC) F80dlacZ Δ M15 | Life Technologies Inc. | LTI‡ catalog, 1999 |
| | | Δ lacX74 deoR recA1 endA1 araD139 Δ(ara, | (Gaithersburg, MD) | |
| | | leu)7679 galU galK1⁻ rpsL nupG | | |
| 168 | B. subtilis | trpC2 (reference strain) | BGSC* | Burkholder et al., |
| | | | | 1947, Amer. J. Bot. |
| | | | | 35: 345 and Kunst et |
| | | | | al., 1997, Nature 390: |
| | | | | 249-56. |
| BD170 | B. subtilis | thr-5, trpC2 | BGSC* | Dubnau et al., 1973, |
| | | | | J. Bacteriol. 114: |
| | | | | 273-86. |
| NO-227 | B. subtilis | BD170, thrC::Cm^{R} | Deposit no. | N/A |
| | | | PTA-1928 | |
| NO-360 | B. subtilis | BD170 thrC::Pspac-murA kan^{R} | Deposit no. | N/A |
| | | | PTA-1937 | |
| NO-347 | B. subtilis | BD170 thrC::Pspac-murA kan^{R} Δ murA::Cm^{R} | Deposit no. | N/A |
| | | | PTA-1939 | |

TABLE 4
Plasmids

| Plasm id | Description | Source | Reference/cat # |
|---|---|---|---|
| pTP223 | P_{lac}-bet exo Tc^{R} | Keenan Murhpy | Murphy, 1998. J. Bacteriol. 180: |
| | | | 2063-71 |
| pTPKm | pTP223, Tc^{R} gene replaced with Km^{R} gene | GTC plasmid | N/A |
| pBC SK | Cloning vector, Ap^{R} | Stratagene; Cat. No. 212215 | |
| pBAD/Myc- | Expression vector. Ap^{R} arabinose P_{BAD} | Invitrogen; Cat. No. V440-01 | |
| HisB | promoter | | |
| pPROLar.A122 | Expression vector, | Clontech; Cat. No. 6220-1 | |
| pPROTet.E232 | Expression vector, | Clontech; Cat. No. K1624-1 | |
| pBL21PRO | Plasmid from BL21PRO. tetR P_{lacI}^{Q}-lacI | Clontech; Cat. No. K1624-1 | |
| | Sp^{R} on an unspecified autonomously | | |
| | replicating plasmid. | | |
| pNK2887 | Mini-Tn10 vector carrying Km^{R} gene | Nancy Kleckner | |
| pGTC302 | pBC.SK yibD::(arabinose p_{BAD}-folA Ap^{R}) | GTC-EC#302 | N/A |
| pGTC318 | pBC.SK yibD::(arabinose P_{lac/ara-1} MCS | GTC-EC#318 | N/A |
| | Ap^{R}) | | |
| pGTC321 | pBC.SK yibD::(arabinose P_{LtetO-1} MCS | GTC-EC#321 | N/A |
| | Ap^{R}) | | |
| pDG148 | Pspac promoter, shuttle plasmid, Ap^{R} | T. Kenney, GTC | Stragier et al., 1988, Cell 52: 697- |
| | (gram neg.), | | 704. |
| pDG1664 | thrC integration vector | BGSC | Guerot-Fleury et al., 1996, Gene 180: |
| | | | 57-61. |
| pHS874 | pMCS5-kanR | M. Sulavik, GTC | N/A |
| pGL3 | Source of kanR | J. Kok | J. Kok, “Special-purpose vectors for |
| | | | lactococci, IN GENETICS AND |
| | | | MOLECULAR BIOLOGY OF |
| | | | SPREPTOCOCCI, LACTOCOCCI, AND |
| | | | ENTEROCOCCI 98 (G.M. Dunny et |
| | | | al., eds. ASM Press 1991) |
| pEVP3 | Source of CmR | D. Morrison | Claverys et al., 1995 Gene 164: 123-8. |
| pMCS5 | Cloning vector | MoBiTec, Gottingen, Germany | MoBiTec catalog, 1999 |

‡LTI, Life Technologies, Inc.
*BGSC, Bacillus Genetic Stock Center, Ohio State University, Columbus, OH)
Wetman et al. (1986) Gene 49: 253-262

The chromosomal locus attB::P_{N25}tetR, P_{laci}^{q}/laci, Sp^{r}, encoding elements for stable expression of the laci repressor and tet repressor, was transduced from DH5αPRO into E coli strains MC1061 and KM354 using P1 transduction, essentially as described in Silhavy et al., EXPERIMENTS WITH GENE FUSIONS, 107-111 (Cold Spring Harbor Press, 1984). pTPKm was constructed by replacing the tetracycline resistance cassette on the plasmid pTP223, carrying the IPTG-inducible recombinogenic function of red/gam, with the kanamycin resistance gene from pNK2887. The resulting variations of pTP223 were electroporated into either MC1061 or KM354 E. coli, and competent and recombinogenic host cells were made as described in Murphy, 1998, J. Bacteriol. 180: 2063-71.

E. coli genes folA and murA were amplified from the genomic DNA of KM29 using the PCR and oligonucleotide primers pairs murA cIF+murA HIScIR and folA cIFnco+folA HIScIR, respectively (see Table 5). Hexahistidine encoding tags were incorporated into the 3′ end of the gene encoding the C-terminus of the protein through primer design.

PCR products for murA and folA were digested with Kpn I-Xba I and Nco I-Xba I, respectively, and cloned into the following expression vectors: pBAD/Myc-His B, pPROTet.E232, and pPROLar. The entire vector minus the origin of replication for each of the resulting recombinant plasmids was amplified using PCR and the following primer pairs (see Table 5): pBAD/Myc-HisB (pBAD F1+pBAD R1), pPROTet.E232, and pPROLar.A122. The resulting PCR products were then linked at their 5′ ends to the 1 kb region upstream of yibD locus amplified using primers yibD F1 a and yibD R2, and linked at their 3′ ends to the 1 kb region downstream of E. coli yibD locus which was amplified using primers yibD F2a and yibD R2 (in effect, replacing yibD) by “crossover PCR” (Kim et al., 1996, Biotechniques, 20: 954-5).

Since the crossover PCRs were not always robust, universal cloning templates in the vector backbone of pBC-SK+/− were also generated that carried 1 kb of DNA 5′ to yibD, arabinose, a regulatable promoter (either P_{BAD}, P_{LtetO-1}, or P_{laci/ara-1}), either MCS (multiple cloning sites) or the folA gene, the ampicillin resistance gene, and 1 kb of DNA 3′ to yibD (See FIG. 2).

PCR products consisting of 1 kb upstream yibD, araC regulatable promoter (either P_{BAD}, P_{LtetO-1}, or P_{lac/ara-1}), gene of interest (either folA gene or murA), the ampicillin resistance gene and 1 kb downstream of yibD were electroporated into competent and recombinogenic E. coli host cells. Transformed colonies carrying one integrated construct were verified by PCR to contain the second copy of either murA or folA downstream of a regulatable promoter in place of yibD. The cells were made competent and recombinogenic again. Linear PCR products that consisted of upstream and downstream regions of the gene of interest linked to a resistance cassette (in essence, the gene of interest is replaced by a resistance cassette) were introduced, and colonies selected in the presence of regulator. The resultant colonies plated in the presence of regulator were verified by diagnostic PCR for deletion of the wild-type gene of interest. DNA oligonucleotide primers used in this work are described in Table 5.

TABLE 5
Primers used in P_{BAD}murA::yibD and P_{BAD}folA::yibD strain construction
All primers are 5′ to 3′

| Cloning primers | Sequence | Description |
|---|---|---|
| murA cIF | GGG GTA CCC ATG GAT AAA TTT CGT GTT CAG GGG | Used to clone murA into |
| | | P_{BAD} vector. Includes |
| | | start of gene and KpnI |
| | | site for cloning. |
| murA HIScIR | CCG GAA TTC TAG ATT A GTG GTG GTG GTG GTG | Used to clone murA into |
| | GTG TTC GCC TTT CAG ACG CTC AAT | P_{BAD} vector. Includes HIS |
| | | tag, stop of gene and XbaI |
| | | site for cloning. |
| folA cIFnco | GGG GTA CCC ATG GTC AGT CTG ATT GCG GCG TTA | Used to clone folA into |
| | | P_{BAD} vector. Includes start |
| | | of gene and NcoI site for |
| | | cloning. |
| | | Note: This primer changes |
| | | 2nd aa from ATC to GTC in |
| | | order to have NcoI site. |
| F1a. HIScIR | CCC AAG CTT CTA GA TTA GTG GTG GTG GTG GTG | Used to clone F1a. into |
| | GTG CCG CCG CTC CAG AAT CTC AAA | P_{BAD} vector. Includes |
| | | HIS tag, stop of gene and |
| | | Bai site for cloning. |
| yibD primers | | Description |
| yibD F1 | GGC CAT ATC ACC TGT GGT CA | Primer upstream of yibD |
| | | F1a. Used for targeting |
| | | PCR to verify insertions in |
| | | yibD locus. |
| yibD F1a | CCA TTT GTG CCG CAA CAC GAT AGG | Used in combination with |
| | | yibD R2. Makes up 5′ |
| | | arm for crossing into yibD |
| | | locus. |
| yibD F2 | TGT GAG CGG ATA ACA ATT TCA CAC AGG AAA ACA | Used in combination with |
| | AAA CGC GGT CGC ACC ATC GCA | R1a. Has tag to KAN F1 (cmr |
| | | tag) primer for determining |
| | | essentiality of yibD. |
| yibD R1 | ATT CGC CGC CAG GGT CAG GC | Primer downstream of yibD |
| | | R1a. Used for targeting |
| | | PCR to verify insertions in |
| | | yibD locus. |
| yibD R1a | CAC AGC CGT GGT TGG GAA GAT AAG | Used in combination with |
| | | yibD F2. Makes up 3′ |
| | | arm for crossing into |
| | | yibD locus. |
| yibD R2 | GTG CGT AAC GGC AAA AGC ACC GCC GGA CAT ACA | Used in combination with |
| | CTA AGT TTA TTG GTG CTG TTC | yibD F1a. Has tag to KAN |
| | | R1 primer for determining |
| | | essentiality of yibD. |
| pBAD/Myc-HisB | | |
| primers | | Description |
| pBAD Forward | ATG CCA TAG CAT TTT TAT CC | Used in combination with |
| | | pBADReverse to verify |
| | | insertion into pBADvector. |
| pBAD Reverse | TCT GAT TTA ATC TGT ATC AGG CTG | Used in combination with |
| | | pBADForward to verify |
| | | insertion into pBADvector. |
| pBAD F1 | TAT GAT GAA CAG CAC CAA TAA ACT TAG TGT CGT | Used in combination with |
| | CTC CGG GAG CTG CAT GTG TCA | pBADR1. Amplifies entire |
| | | pBADvector except for |
| | | origin. Has tag to yibDR2. |
| pBAD R1 | GCG TAA TGC GAT GGT GCG ACC GCG TTT TGT AAC | Used in combination with |
| | TCA CGT TAA GGG ATT TTG GTC | pBADF1. Amplifies entire |
| | | pBADvector except for |
| | | origin. Has tag to yibDF2. |

Note:
PCR product pBADF1/pBADR1 used in crossover PCR with yibD
F1a/R2 and yibD R1a/F2 to link all 3 pieces to be used to
cross into yibD locus.

| Knock-out | | |
|---|---|---|
| primers | | |
| murA | | Description |
| murA F1 | GCG TTA CCA TTA TTG ACC CGA ATG | Used in combination with |
| | | murA R2. Makes up 5′ arm |
| | | for crossing into murA |
| | | locus. |
| murA F2 | TGT GAG CGG ATA ACA ATT TCA CAC AGG AAA GCA | Used in combination with |
| | AAT ATT GAG CGT GTG AAA GGC | murA R1. Has tag to KAN F1 |
| | | (cmr tag) primer for |
| | | knocking out wild-type copy |
| | | of murA. |
| murA F3 | CTG CTC AAC GTG AAG CCT ACT TTG | Primer upstream of murA F1. |
| | | Used for targeting PCR to |
| | | verify insertions into murA |
| | | locus. |
| murA R1 | CTA CAT GCG CGT TAT CTA TAG CAA | Used in combination with |
| | | murA F2. Makes up 3′ arm |
| | | for crossing into murA |
| | | locus. |
| murA R2 | GTG CGT AAC GGC AAA AGC ACC GCC GGA CAT CGT | Used in combination with |
| | TGG CCC CTG AAC ACG AAA TTT | murA F1. Has tag to KAN R1 |
| | | primer for knocking out |
| | | wild-type copy of murA. |
| murA R3 | CAT GTC AGA AGC CGT AAA CGT CAT | Primer downstream of murA |
| | | R1. Used for targeting PCR |
| | | to verify insertions in |
| | | murA locus. |
| folA | | Description |
| folA F1 | TGT TAG GGC AGG GCA GTG AGT TTG | Used in combination with |
| | | folA R2. Makes us 5′ arm |
| | | for crossing into folA |
| | | locus. |
| folA F2 | TGT GAG CGG ATA ACA ATT TCA CAC AGG AAA TAT | Used in combination with |
| | TGC TTT GAG ATT CTG GAG CGG | folA R1. Has tag to KAN F1 |
| | | (cmr tag) primer for |
| | | knocking out wild-type copy |
| | | of folA. |
| folA F3 | CTC ATC ATC AAA ATC GCC ATG CTG | Primer upstream of folA F1. |
| | | Used for targeting PCR to |
| | | verify insertions into folA |
| | | locus. |
| folA R1 | CCA TGC AGG GTC ACT ACG AAA TGA | Used in combination with |
| | | folA F2. Makes up 3′ arm |
| | | for crossing into folA |
| | | locus. |
| folA R2 | GTG CGT AAC GGC AAA AGC ACC GCC GGA CAT | Used in combination with |
| | TAC CGC TAA CGC CGC AAT CAG ACT | folA F1. Has tag to KAN R1 |
| | | primer for knocking out |
| | | wild-type copy of folA. |
| folA R3 | ATA CCG TAA CCA TAC GCA ATC TGG | Primer downstream of folA |
| | | R1. Used for targeting PCR |
| | | to verify insertions in |
| | | folA locus. |
| Kanamycin | | |
| primers | | |
| (pNK2887) | | Description |
| Kan F1 (cmr | ATG TCC GGC GGT GCT TTT GCC GTT ACG CAC AAG | Used in combination with |
| tag) | CCA CGT TGT GTC TCA AA | Kan R1. Amplifies Kan |
| | | cassette including Kan |
| | | promoter and PLAC-UV5 |
| | | promoter. |
| | | Note: This primer contains |
| | | tag to CMR F1 (cassette |
| | | from pNK2884) so same ′gene |
| | | R2's can be used for either |
| | | cassette. |
| Kan R1 | GAA ATT GTT ATC CGC TCA CA | Used in combination with |
| | | Kan F1. Amplifies Kan |
| | | cassette including Kan |
| | | promoter and PLAC-UV5 |
| | | promoter. |
| Kan ck1 | ATT GGT TGT AAC ACT GGC AGA GCA | Used in combination with |
| | | reverse targeting primers |
| | | to verify wild-type copy of |
| | | gene has been knocked out. |
| Kan ck2 | ATC ATC AGG AGT ACG GAT AAA ATG | Used in combination with |
| | | forward targeting primers |
| | | to verify wild-type copy of |
| | | gene has been knocked out. |

Note:
This cassette contains a pLAC-UV5 outward reading promoter.

The specificity and sensitivity of the resultant colonies to added drugs were followed by measuring the minimal inhibitory concentration (MIC) of trimethoprim, sulfamethozasone, phosphomycin and D-cycloserine at varying regulator levels. This was performed according to NCCLS (National Center for Clinical Laboratory Standards) recommendations(NCCLS, 1997, METHODS FOR DILUTION ANTIMICROBIAL SUSCEPTIBILITY TESTS FOR BACTERIA THAT GROW AEROBICALLY, 4th ed.; approved standard. NCCLS document M7-A4. NCCLS, Wayne, Pa.), in 100 μl volume in 96-well format, with serial two-fold dilutions of drug concentration.

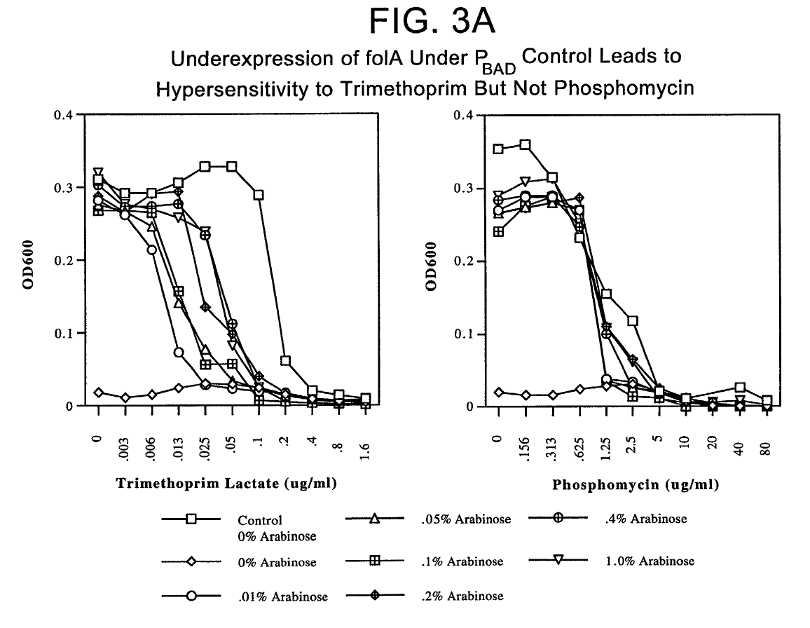

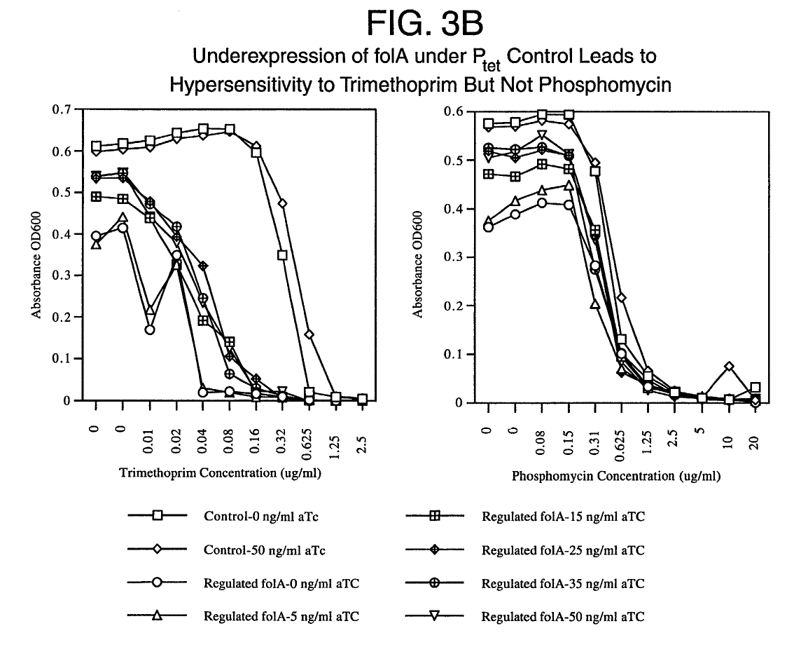

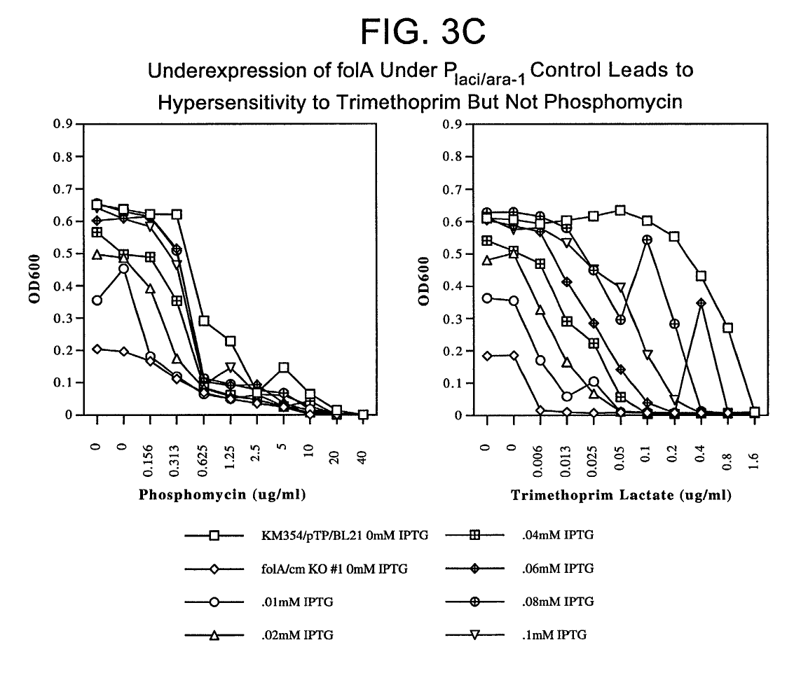

Results. In cells in which folA is regulated by P_{BAD}, P_{LtetO-1}, or P_{lac/ara-1}, the MIC of trimethoprim decreases with decreasing regulator (L-arabinose, anhydrotetracycline, and IPTG, respectively) level, whereas MIC of phosphomycin is relatively unchanged as seen in FIG. 3.

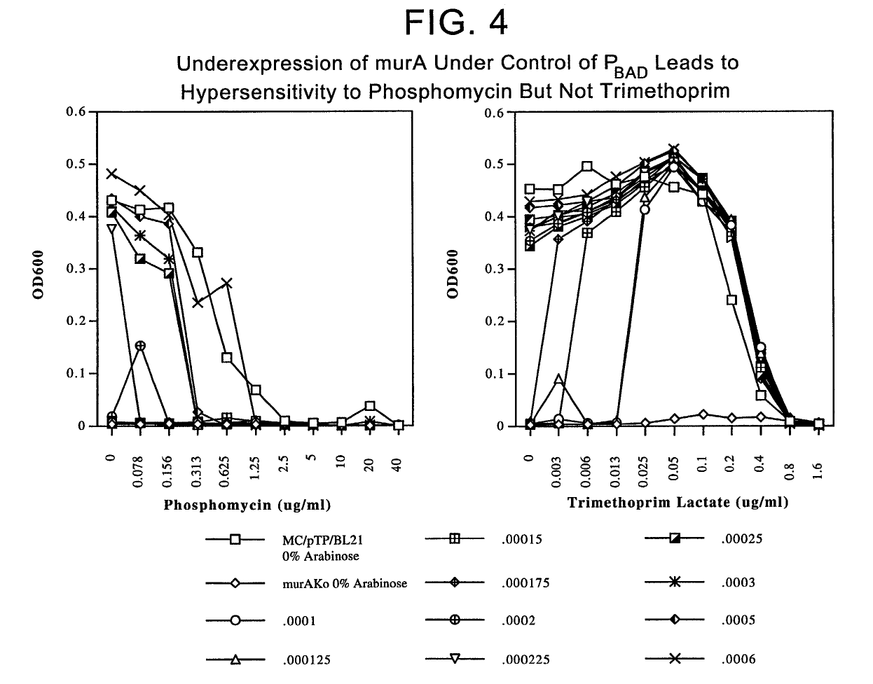

FIG. 4a shows that for cells in which mura is regulated by P_{BAD}, the MIC of phosphomycin decreases with decreasing regulator (L-arabinose) level, whereas the MIC of trimethoprim is relatively unchanged.

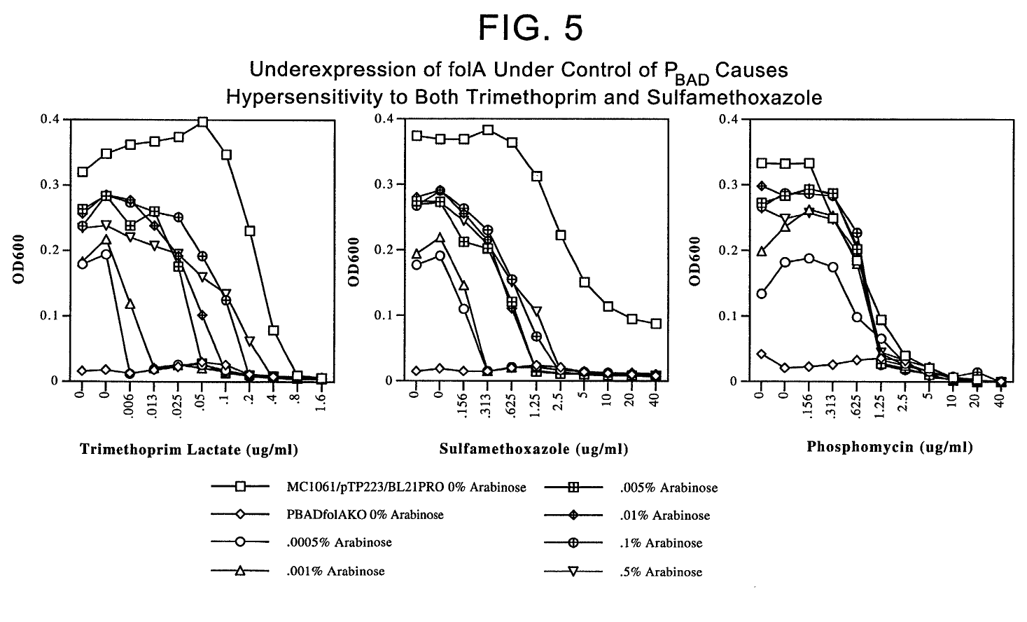

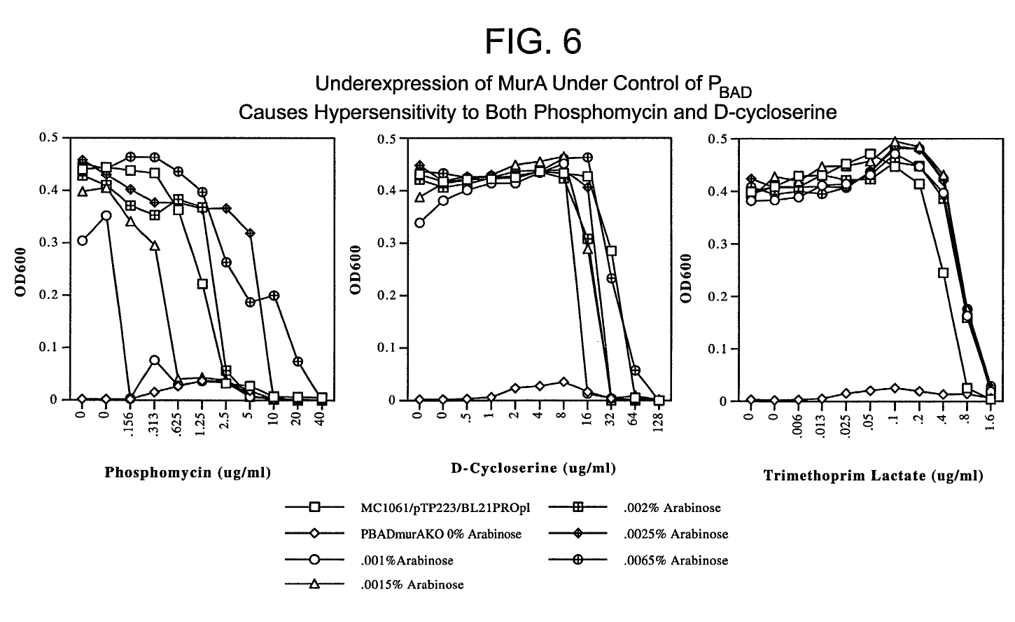

FIG. 5 shows that for cells in which folA is regulated by P_{BAD}, the MIC of both trimethoprim and sulfamethozasone decreases with decreasing regulator (L-arabinose) level. Trimethoprim and sulfamethozasone are two different antibiotics that act at different steps of the folate biosynthesis pathway.

### Example 2

### Cell Assay Using Under-expression of murA in B. subtilis

A cell assay system has also been set up to study antimicrobials and other agents in a gram-positive bacterial environment. In this example, the wild-type gene for murA was knocked out and the gene replaced under ectopic expression in the thrC locus, under regulation by the P_{spac} promoter with IPTG (isopropyl β-D-thiogalactoside).

Materials and Methods. The materials are the same as discussed above. Bacterial strains are listed in Table 3. Plasmids are listed in Table 4 B. subtilis strains were routinely grown in LB media or on LB agar plates containing 1.5% agar (Sambrook et al., MOLECULAR CLONING: A LABORATORY MANUAL, 2nd ed., Cold Spring Harbor Laboratory Press, 1989). MIC determination is performed using microbroth dilution-cultures grown in Mueller Hinton broth in 96-well microtiter plates purchased from Nunc (Plates—Cat. No. 262162; Lids—Cat. No. 264122). When required, media were supplemented with 5 μg/ml chloramphenicol or 10 μg/ml kanamycin. IPTG was added to culture media at a final concentration of 0.05 mM for routine culture growth of B. subtilis NO-347 (BDI70 thrC::Pspac-murA kan^{R} ΔmurA::Cm^{R}), which is dependent on IPTG for growth.

PCR was used to amplify target DNA from genomic DNA prepared from B. subtilis 168 and plasmid DNAs listed in Table 6. PCR reactions contained 0.2 μM primers, 10 ng template DNA and 45 μl PCR Superimix High Fidelity (Life Technologies, Inc.; Cat. No. 10790-020). Custom DNA oligonucleotide primers were obtained from Life Technologies Inc. (Gaithersburg, Md.). The DNA oligonucleotide primers used in the described work are listed in Table 6.

Crossover PCR was used to link PCR products together and was performed essentially as described in Kim et al., 1996, Biotechniques 20: 954-5. Briefly, PCR primers used to amplify individual PCR products contained sequences that would hybridize to the end of the fragment to be linked (30-40 bp). The resulting PCR products were mixed together with primers that amplify the final, linked product (primers that hybridize to the distal 5′ and 3′ ends of the final product).

TABLE 6

Primers used in B. subtilis project

| | |
|---|---|
| Sequence (5′→3′) | Description |

| | | |
|---|---|---|
| Primer | | |
| name | | |
| NO-9 | GTACGCAGTGATGTTTCCAGCATTTCCGA | pPn-lacI F1- |
| | CGCGGCCGCGGAGCTGCATGTGTCAGAGG | thrC(B) |
| | TTTT | |
| NO-10 | AGCCCCACGAAGGAGGTGAGGAAGTAGAA | Ppn-lacI R1- |
| | GAAACGAGGTCATCATTTCCTTCCG | tagRC |
| NO-11 | CTTCTACTTCCTCACCTCCTTCGTGGGGT | Pspac F1-tag |
| | CTACACAGCCCAGTCCAGACTATTC | |
| NO-12 | TTTGAGTGCCTCCTTATAATTTATTTTGT | Pspac R1-RBS |
| | AGTTCCTTCGAAGCTTAATTGTTATCCGC | |
| | TCACAA | |
| NO-22 | TCGAAGGAACTACAAAATAAATTATAAGG | murA F1-RBS |
| | AGGCACTCAAATTGGAAAAAATCATCGTC | |
| | CGCGGCG | |
| NO-23 | TTATTGTCCTGGGTTTCAAGCATTAGTTC | murA R1-kan |
| | CAGGCCGGCCTGCCAGAGTGACATACTGA | |
| | TTTTC | |
| NO-31 | TTTGGCGCGCCTTTGGCCGGCCTTTGCGG | thrC-B R1-enz |
| | CCGCGTCGGAAATGCTGGAAACATCACT | tag |
| NO -32 | TTTGGCCGGCCTTTGGCGCGCCTAATTAA | thrC-F F1-enz |
| | TTAATGCGGTAAGGGTTGACTGAGTTGA | tag |
| NO-42 | CCCTTGTCAACTCAGTCAACCCTTACCGC | CmR Lower- |
| | AATAAGCTTGATGAAAATTTGTTTG | thrC (F) |
| NO-43 | GTACGCAGTGATGTTTCCAGCATTTCCGA | CmR Upper- |
| | CGGGGCAGGTTAGTGACATTAGAAA | thrC (B) |
| NO-53 | AATTCCGGCGACTGTTTCTGTTTC | thrC back F2 |
| | | (pDg1664) |
| NO-54 | CATGTAAAAGATGAGGTTGGTTCA | thrC front R2 |
| | | (pDg1664) |
| KO | | |
| primer | | |
| F1 | TAAAAATCAAACAAATTTTCATCAAGCTT | |
| KO murA | ATTTTGAC | |
| R2 | TGTGCCGTTTAACTTCTG | |
| KO murA | AGTCGGTTTTCTAATGTCACTAACCTGCC | |
| | CCCGAGCA | |
| F2 | AGAGAATAAAGAAGTCGTT | |
| KO murA | | |
| R1 | CTCACCATATTGCTTTTCAGGCTG | |
| KO murA | | |
| F3 | TGATTTCCACCTAACGCCCCTATG | |
| KO murA | | |
| R3 | GGGGCAGGTTAGTGACATTAGAAA | |
| Cm-U | | |
| Cm-L | ATAAGCTTGATGAAAATTTGTTTG | |

Wherein “KO” stands for knock-out.

Plasmid DNA was introduced into electrocompetent E. coli DH10B by electroporation. Electrocompetent E. coli DH10B (Cat. No. 18290-015) were purchased from Life Technologies, Inc. and used according to manufacturer's directions. Linear and plasmid DNA was introduced into naturally competent B. subtilis BD170. Induction of natural competence in B. subtilis BD170 and transformation using frozen competent cells was performed as described in Cutting et al., “Genetic Analysis,” IN MOLECULAR BIOLOGICAL METHODS FOR BACILLUS 27-60 (C. R. Harwood et al., eds. John Wiley & Sons Ltd., 1990). Molecular cloning/DNA manipulations procedures were performed according to Sambrook et al., MOLECULAR CLONING: A LABORATORY MANUAL (2nd ed., Cold Spring Harbor Laboratory Press 1989).

The following restriction enzymes (catalog numbers in parentheses) were purchased from New England Biolabs (Beverly, Mass.) and were used according to manufacturers instructions: Fse I (Cat. No. 588L), Not I (Cat. No. 189L), Asc I (Cat. No. 558L), and Pac I (Cat. No. 547L). T4 DNA ligase and reaction buffer (Cat. No. 716 359) were purchased from Boerhinger Mannheim Biochemicals (Indianapolis, Id.) and were used according to manufacturer's instructions. Plasmids used in this work are listed in Table 3, supra. Plasmid DNA was prepared using mini- and midi-plasmid preparation kits from Qiagen Inc. (Valencia, Calif.) according to manufacturer's instructions.

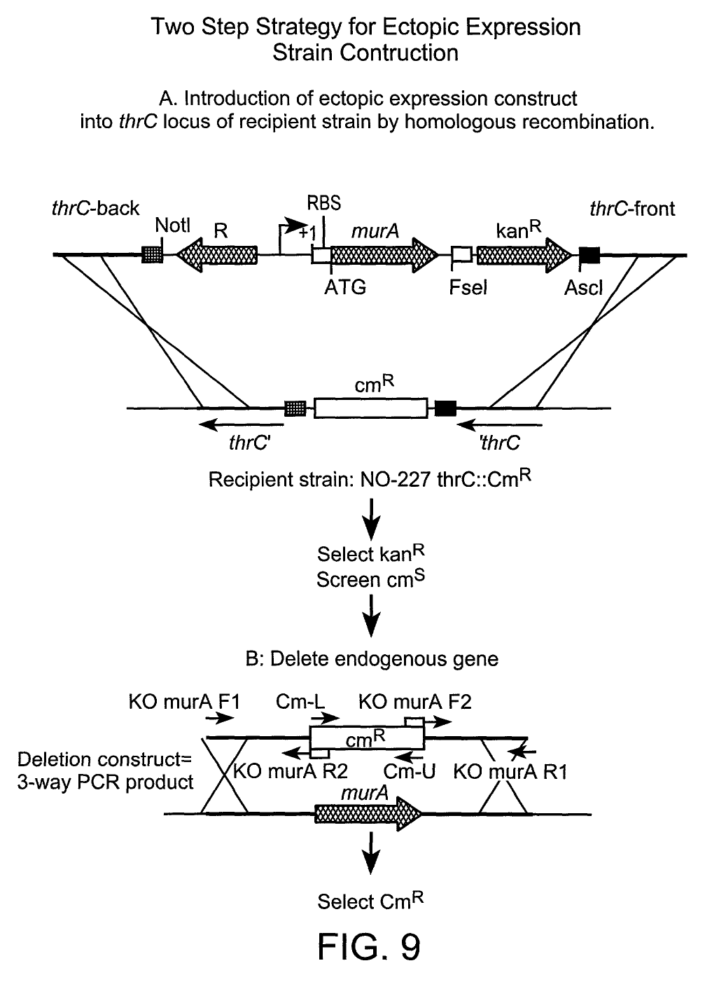

Construction of B. subtilis NO-347 (BDI70 thrC::Pspac-murA kan^{R} ΔmurA::Cm^{R}) ectopic expression strain. The overall strategy for construction of a Pspac-murA kan^{R} ectopic expression strain is outlined in FIG. 9.

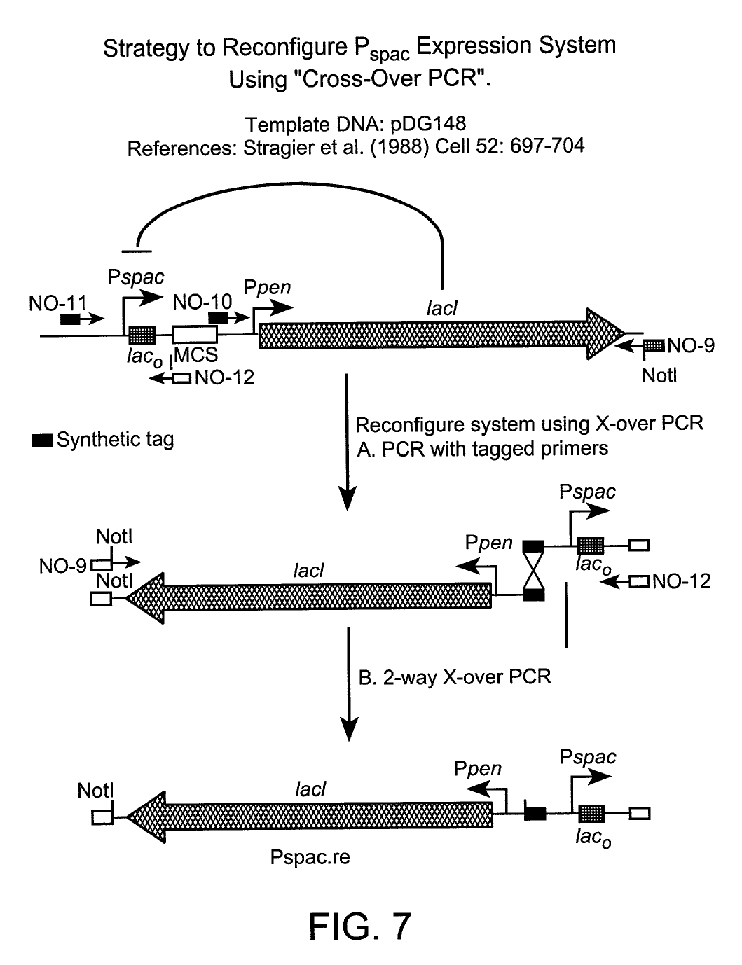

The Pspac expression system (Yansura et al., 1984, Proc. Natl. Acad. Sci. USA 81: 439-43) is shown in FIG. 7. It consists of a hybrid B. subtilis phage SPO-1 promoter and the E. coli lac operator, designated spac-1, and of the E. coli lad gene, encoding lac repressor, expressed by Ppcn of B. licheniformis. Expression of Pspac can be regulated by supplementing the culture medium with IPTG. The Pspac expression system was reconfigured as shown in FIG. 7 to produce Pspac.re, which results in an arrangement in which Ppen-lacI and Pspac are transcribed divergently and allows for essential genes to be linked to Pspac.re by crossover PCR. Briefly, the PCR was used to amplify a DNA fragments containing Ppen-lacI and Pspac from pDG148 template DNA using DNA oligonucleotide primer pairs NO-9 and NO-10, and NO-11 and NO-12, respectively. The crossover PCR technique (Kim et al., 1996) with primer pair NO-9 and NO-12 was used to generate the reconfigured Pspac expression system shown at the bottom of FIG. 7. The structure of Pspac.re was confirmed by sequence analyses.

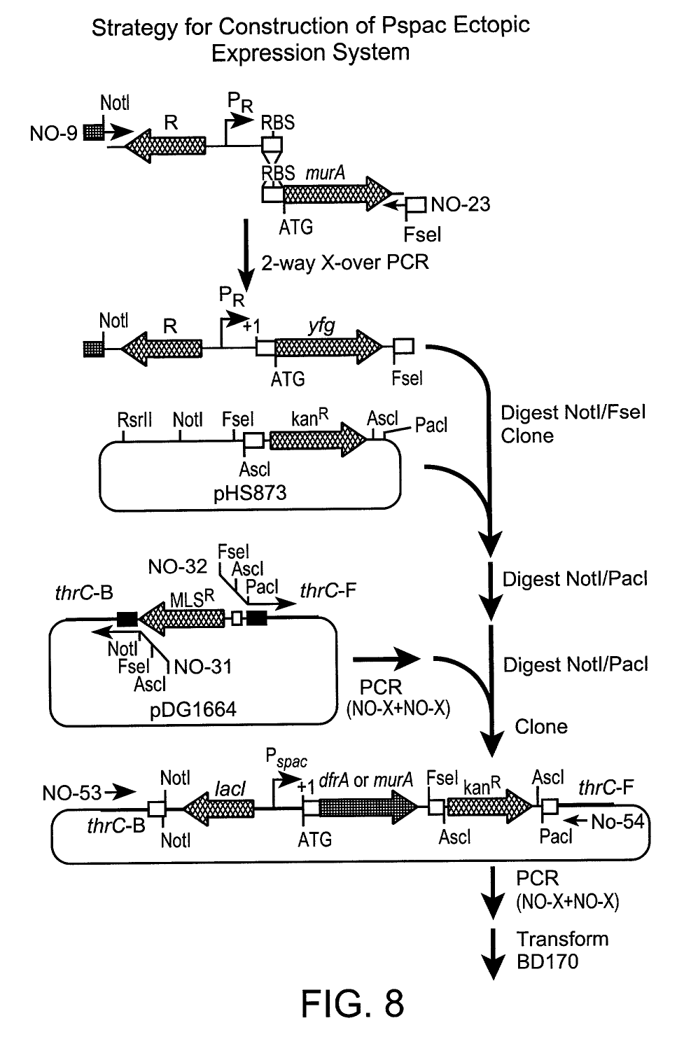

The construction of the Pspac-murA ectopic expression construct is outlined in FIG. 8. PCR was used to amplify the B. subtilis murA gene from B. subtilis 168 genomic DNA using primer pair NO-22 and NO-23. Crossover PCR was used to link Pspac.re to the amplified murA gene using primer pair NO-9 and NO-23. The resulting PCR product was digested with Not I and Fse I and was ligated with pHS873 previously digested with Not I and Fse I. The ligation mixture was used to transform electrocompetent E. coli DH10B cells. Resulting kanR colonies were screened for presence of Pspac.re-murA insert. Plasmid DNA was prepared from a verified clone, digested with Not I and Pac I, and the restriction fragment containing Pspac.re-murA kanR was gel-purified. This DNA fragment was ligated with a Not I-Pac I-digested PCR product that was generated using pDG1664 template DNA and primer pair NO-31 and NO-32. The resulting ligation mixture was used to transform electrocompetent E. coli DH10B cells. KanR clones were selected and were further screened for the presence of Pspac.re-murA and thrC(F) and thrC(B) using PCR. The final ectopic construct was amplified using the PCR with primer pair NO-53 and NO-54. The resulting PCR product was used to transform naturally competent B. subtilis NO-227. Kan^{R} transformants were selected, which were subsequently screened for Cm^{S} phenotype (see FIG. 9). B. subtilis strain NO-360 (BD170 thrC::Pspac-murA kan^{R} Cm^{S}) was chosen as further strain construction.

The endogenous murA gene in NO-360 was deleted by allelic replacement as follows (see FIG. 9). DNA fragments flanking the 5′ and 3′ ends of the murA gene were amplified from B. subtilis 168 genomic DNA using primer pairs KO mura F1+R2 and KO murA F2+R1, respectively. The 5′ and 3′ flanking fragments were linked to the Cm^{R} gene from pEVP3 using the cross-over PCR technique with the primer pair KO mura F1+R1. The resulting PCR product (shown in FIG. 9B) was used to transform naturally competent B. subtilis NO-360. Cm^{R} clones were selected in the presence of 0.01 mM and 0.05 mM IPTG. Allelic replacement of murA in Cm^{R} clones was verified by PCR. Strain NO-347 (BD170 thrC::Pspac-murA Kan^{R} ΔmurA::Cm^{R}) was chosen for MIC determinations for phosphomycin in presence of various concentrations of the regulator IPTG.

MIC determinations. Determination of minimum inhibitory concentration (MIC) for phosphomycin, novobiocin, erythromycin and trimethoprim lactate were performed in 96-well microliter plates using the microbroth dilution technique as described in NCCLS. 1997. Methods for dilution antimicrobial susceptibility tests for bacteria that grow aerobically-fourth edition; approved standard. NCCLS document M7-A4. NCCLS, Wayne, Pa. For MIC determinations using strain NO-347, various concentrations (0-0.1 mM) were added to the media. Growth was quantitated by OD₆₀₀ measurements using a SpectroMax 250 plate reader (Molecular Devices).

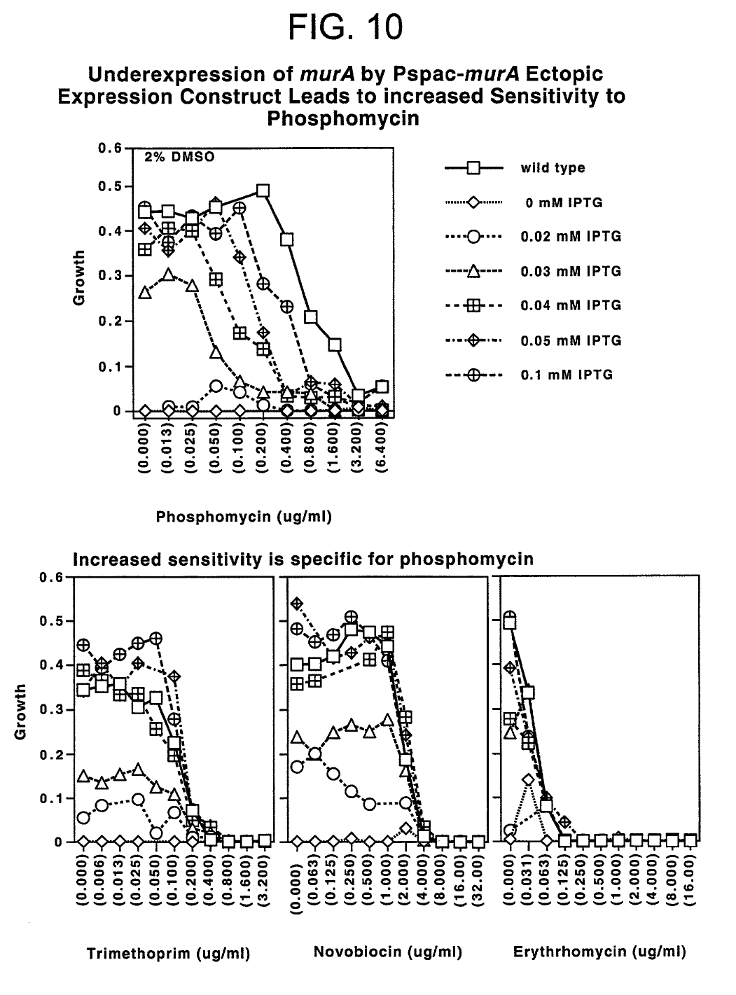

Results. Results of MIC analyses demonstrate that regulated under-expression of murA results in increased sensitivity to phosphomycin compared to the control strain (FIG. 10). Regulated under-expression of murA does not affect sensitivity to trimethoprim, novobiocin, or erythromycin, indicating that the increased sensitivity is specific for phosphomycin.

### Example 3

### Identification of a gene using GARIT: Transformation Selection to Identify the Gene Target of murA and of folA

Materials. Bacterial strains. The bacterial strains E. coli MC1061[F−araD139 D (ara-leu)7696 galE15 galK16 D(lac)X74 rpsL (Str^{r}) hsdR2 (r_{K}^{-m}_{K}⁻) mcrA mcrB1]were employed in this experiment.

Plasmids. The plasmids pTP223 (from Kenan Murphy, University of Massachusetts) and pBAD/Myc-HisB vector (from Invitrogen, Cat. No. V440-01) were employed for this experiment.

Media and Chemicals. The following media and chemicals were utilized: Mueller Hinton Broth (Difco Cat. No. 0757-17-6), Trimethoprim Lactate (Sigma Cat. No. T-0667), Phosphomycin (Sigma Cat. No. P-5396), Ampicillin (Sigma Cat. No. A-9518), Tetracycline (Sigma Cat. No. T-3383) and L (+) Arabinose (Sigma Cat. No. A-3256).

Methods. The genes folA, and murA were amplified from the genomic DNA of E. coli. Each gene was independently cloned in frame into pBAD/Myc-HisB vector, such that expression of the gene is under the regulation of the promoter P_{BAD}. Hexahistidine encoding tags were incorporated into the 3′ end of the gene encoding the C-terminus of the protein through primer design. The construct introduced into the host cells of E. coli MC1061, resulting in Ec211 (construct with folA) and Ec261 (construct with murA).

The entire vector region (araC, P_{BAD}, folA or murA, ampicillin resistance gene) of the above two constructs, but minus the origin, was PCR amplified through primer pairs homologous to regions flanking the origin. The PCR products were then linked at their 5′ ends to the 1 kb region upstream of yibD locus, and linked at their 3′ ends to the 1 kb region downstream of E. coli yibD locus (in effect, replacing yibD) by “crossover PCR” (Kim et al., 1996 Biotechniques 20: 954-5). The linear DNA product was gel purified and introduced into competent and recombinogenic MC1061. Recombinogenic ability was supplied by the red/gam function of plasmid pTP223. Successful recombination of the linear piece resulted in Ec239, cells with two copies of folA, one at the natural locus of folA under wild-type regulation and the second copy at the yibD locus regulated by the arabinose P_{BAD} promoter and in Ec238, where the gene of interest is murA.

Ec211, Ec216, Ec238, Ec239, and MC1061 were freshly streaked onto Mueller Hinton Broth (MHB) plates. After an overnight incubation at 37° C., they were streaked into different segments onto different selection plates in the order of MHB supplemented with 5 μg/ml trimethoprim, MHB supplemented with 5 μg/ml trimethoprim and 0.1% L-arabinose, MHB supplemented with 25 μg/ml phosphomycin, MHB supplemented with 25 μg/ml phosphomycin and 0.1% L-arabinose, MHB supplemented with 0.1% L-arabinose, and finally plain MHB plates. The plates are 10 cm circular plates filled with 30 ml of media and divided into 6 equal segments. MC1061 was streaked in the first segment, Ec211 in second, Ec261 in third, Ec239 in fourth, Ec238 in fifth and the sixth segment was left blank.

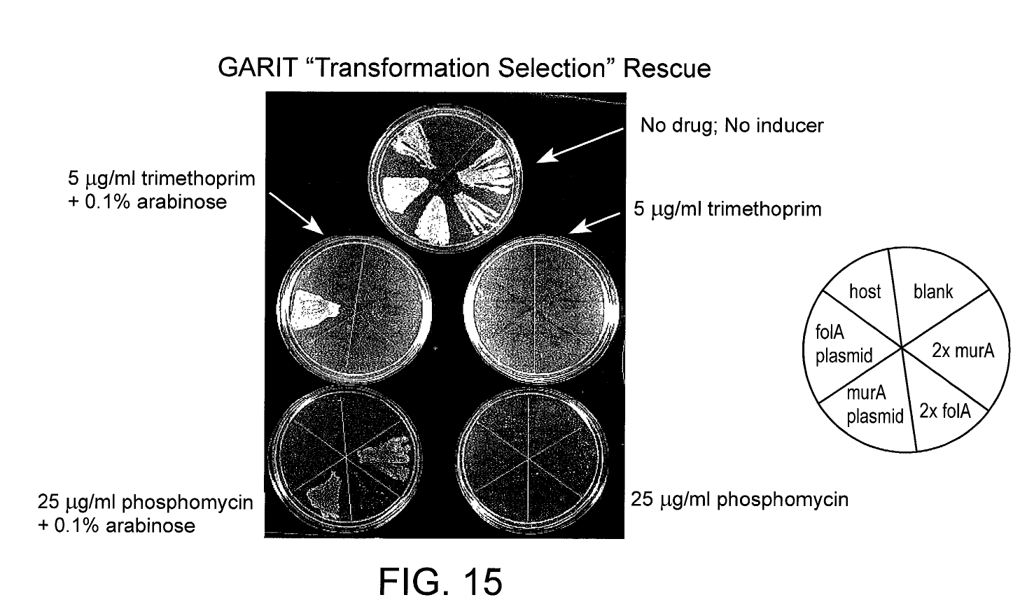

Results. Nothing grew in MHB plates supplemented with 5 μg/ml trimethoprim or with 25 μg/ml phosphomycin (see FIG. 15). On MHB plates supplemented with 5 μg/ml trimethoprim and 0.1% L-arabinose, Ec211 (multiple copies of folA regulated by the arabinose P_{BAD} promoter) was the only strain that grew up. On MHB plates supplemented with 25 μg/ml phosphomycin and 0.1% L-arabinose, both Ec261 (multiple copies of murA regulated by the arabinose P_{BAD} promoter) and Ec238 (two copies of murA, one at the natural locus of murA under wild-type regulation and the second copy at the yibD locus regulated by the arabinose P_{BAD} promoter) grew up. On MHB plates and MHB supplemented with 0.1% L-arabinose, there was growth in every segment except in the sixth segment where no cells had been applied.

### Example 4

### Microarray Analysis of Staphylococcus epidermidis Cells

### Growth of Staphylococcus epidermidis Cells in the Presence of Antibiotics.

S. epidermidis cells from colonies on agar medium were inoculated into Mueller-Hinton broth and grown with shaking at 37° C. overnight. The overnight culture was diluted about 1:50 or such that the OD₆₀₀ was less than 0.05 and grown further with shaking at 37° C. When the culture density reached about OD₆₀₀=0.2, it was split into two separate flasks (400 ml each). To one flask was added an amount of antibiotic to create a concentration of between 10% and 90% of the MIC (minimal inhibitory concentration, determined previously by standard methods; cite the NCCLS standards as before) but preferably at about 50% of the MIC, and an equal volume of water was added to the other flask. Cells were grown for about an additional 200 minutes, taking samples from both flasks for preparation of total RNA at several time points during the growth (e.g., at 0′, 10′, 30′, 60′, 90′, 120′, 150′ and 180′ ). The volume of sample taken from each culture for each point was based on the OD₆₀₀ value, assuming 10 μg of total RNA are typically obtained from 1 ml of cells at OD₆₀₀=0.45.

To isolate total RNA from S. epidermidis cultures cells, cells were first harvested by 0.2 micron filtration. The cells were then resuspended in a solution comprising 4 M guanidine isothiocynate, 10 mM TRIS (pH=7.5) and 1% β-mercaptoethanol. Cells were lysed using a glass beads procedure in the presence of TRIZOL. The remaining procedures for RNA isolation are essentially the same as suggested by the manufacturer of TRIZOL (Life Technologies, Gaithersburg, Md.)

Probe Preparation. Reverse transcriptions were performed using 10 μg of total RNA for either cy3 or cy5 labeling with the following additional components added to a final volume of 50 μl: 0.1 mM cy3- or cy5-dCTP; IX RT Buffer; 10 mM DTT; 0.5 mM dG,dA,dTTP; 0.2 mM dCTP; 10 U RNase inhibitor; and 5 U/μl SuperScript II reverse transcriptase. The reaction temperature parameters are as follows: 26° C. for 20 min., 37° C. for 100 min., and 70° C. for 20 min. After reverse transcriptase reaction, RNA template in the reaction mixture is digested with RNase A and Rnase H. Labeled probes are purified with QIAQuick™ PCR purification kit according to manufacturer's recommendation (QIAGEN GMBH, Hilden, Germany).

Hybridization and Post-hybridization Wash. The dried probes were resuspended in 11.25 μl 5×SSC. SDS was added to a final concentration of 0.2%. The resuspended probes were incubated at 95° C. for 10 min. before applying the probe to the microarrays. Hybridization was performed at 62° C. overnight in a sealed and humidified chamber. Slides were washed consecutively in the following solutions: 1×SSC/0.1% SDS; 0.1×SSC/0.1% SDS; and 0.1×SSC.

Data collection and analysis. Slides were scanned using a dual-laser scanner (General Scanning) at two different wavelengths sequentially (543 nm for the cy3-labeled untreated control and 637 nm for the cy5-labeled antibiotic-treated sample) to generate two TIFF image files. These TIFF image files were then exported to IMAGENE™ (BioDiscovery, Inc., Los Angeles, Calif.) for data analysis. Raw intensity values (cy3 and cy5) for each spot are extracted using IMAGENE™ software. A “Z score” (log of signal ratio minus the average of the log of signal ratios divided by the standard deviation of the log of signal ratios) for each gene is calculated based on the raw intensity values according to a set of rigorous criteria (e.g., signal must be significantly above that of the negative controls, which consist of Arabidopsis DNA with no significant sequence similarity to S. epidermidis DNA. Genes exhibiting Z scores of >2 or <2 are considered statistically significantly up- or down-regulated, respectively. During treatment with other antibiotics or other conditions (such as growth in stationary phase) reveals that most such treatments cause the up- or down-regulation of a relatively unique set of genes specific for the treatment examined. This unique set of genes is an indication of the mode of action of the antibiotic.

Results. Using the above methods, the results displayed in FIG. 11 were obtained.

### Example 5

### Resistant Mutation Mapping

E. coli mutants resistant to phosphomycin were obtained by plating E. coli MC1061 cells in the presence of 5 μg/ml phosphomycin (about 5×MIC), in MHB. Colonies of these resistant mutants were picked and repatched on MHB plates supplemented with 5 μg/ml. Sequencing of the murA genes from two independent phosphomycin-resistant isolates showed a change at nucleotide 83 from a T to a C (T→C), and at nucleotide 84 from a C to a T (C→T), changing amino acid 28 from isoleucine to asparagine. To determine whether the phenotype of phosphomycin resistance is due to these changes in the murA gene, primers murA-F1 and murA-R1 were used to amplify up a region consisting of 1 kb upstream of the murA gene and 1 kb downstream of the murA gene using as template, DNA extracted from MC1061 and from the phosphomycin-resistant murA mutants. The 3.5 kb PCR amplification fragments were cloned into a high copy vector, and 3 independent clones were picked for each type. The clones, carrying multi-copy plasmids with either wild-type murA or mutated mura, were streaked onto MHB plates and on MHB supplemented with 5, 25, and 50 ,μg/ml phosphomycin. The wild-type MC1061 strain (carrying a single copy of the wild-type murA gene) and the original mutants resistant to phosphomycin were used as controls. All strains grew on MHB plates without phosphomycin. On MHB plates with 5 μg/ml phosphomycin, there was slight growth for strains carrying the wild-type murA gene, whether present in single copy or multiple copies, whereas there was robust growth for cells with the mutated murA gene, whether present in single or multiple copies. On MHB plates with 25 μg/ml phosphomycin, there was no growth for strains carrying the wild-type mura gene, whether present in single copy or multiple copies, but there was robust growth for cells carrying single or multiple copies of the mutated murA gene. On MHB plates with 50 μg/ml phosphomycin, there was no growth for strains carrying wild-type murA (whether present in single copy or multiple copies) but slight growth for strains carrying the mutated murA gene whether present in single or multiple copies. Clearly, the mutants isolated because of their phenotype of resistance to phosphomycin carry mutant mura genes which can confer resistance when transferred to other cells by transformation. Thus, the target of the antibiotic phosphomycin was determined by mapping the site of mutations providing resistance to phosphomycin. Since these mutations are dominant over the wild-type allele, such mapping can be accomplished by transformation of wild-type cells with plasmids carrying the mutant alleles.

### Example 6

### Resistant Mutation Mapping with Increased Mutation Rate by MNNG

The mutation frequency of E. coli was increased by treatment with MNNG (N-methyl-N′ -Nitro-N-Nitroso guanidine). Log-phase cells (OD₆₀₀ of 0.8) were spun down, washed with cold citrate buffer and concentrated to 16 OD₆₀₀units /0.25 ml in cold citrate buffer (100MM citrate buffer, pH 5.5). MNNG was added to a final concentration of 400μg/ml. After 20 minutes of incubation, 5 times volume of cold 100mM sodium phosphate buffer,pH7.0 was added to stop the reaction. The cells were washed once with 1 ml of LB and resuspended in 1 ml LB. Aliquots of cells were plated on LB agar to calculate the survival fraction after treatment with MNNG. They were also plated onto different selection plates which did not support the growth of wild-type cells. The selection plates consisted of LB agar, each supplemented with 2 to 8 fold MIC of nalidixic acid, rifampicin, or triclosan. The mutation frequency to resistance for each antibiotic is calculated as the fraction of cells that can grow to form colonies in the plates supplemented with the antibiotic. There was an increase of mutation frequency to all three antibiotics after MNNG treatment. The mutation frequency increased from <10⁻⁹ to 3.2×10⁻⁸ for triclosan resisatnce, from 3.5×10⁻⁸ to 1.6×10⁻⁶ for nalidixic acid resistance, and from from 3.5×10⁻⁸ to 4.8×10⁻⁶ for rifampicin resistance.

Resistant mutants were picked and repatched to fresh selection plates to check for specificity. In each case, the mutants remained resistant only to the antibiotic from which they were originally selected. Two independent colonies from each antibiotic selection plate were selected for mapping of the mutations responsible for the resistance. The cells were grown and the DNA purified. The DNA was randomly sheared to about 4 kb, the ends healed and ligated to linker (5′ PGTCTTCACCACGGGG 3′ and 5′ GTGGTGAAGAC 3′ ). This product was gel purified, ligated to BstXI digested pGTC vector (Genome Therapeutics Corporation), and electroporated into DH10B. A pBluescriptSKII vector (Stratgene) with cloning sites modified by standard techniques to include the two BstX1 sequences (CCAGCCCCTTGG and CCAAGGGGCTGG) could also be used. Aliquots (˜5×10⁵ cells) were plated onto LB agar supplemented with the antibiotic from which the original colony was selected. Plasmids were purified from resulting resistant colonies. The plasmids were retransformed into fresh hosts to verify that the resistance is carried on the plasmids. and the ends of the insert sequenced.

The sequences were mapped back to the genome by comparing them to the complete DNA sequence for the E. coli genome. For inserts with more than one orf (“open reading frame”), primers were designed for each individual orf, including the promoter region. The primers were used to generate amplicons using as templates, the plasmids carrying the resistance and chromosomal DNA from the sensitive parent. The amplicons were cloned using the TA cloning kit (Invitrogen K4560-01). The resulting plasmids were first verified for resistance, before the entire insert was sequenced. The sequences from the resistant clones were checked for deviation from the wild-type sequences to characterize the mutations. Mutations detected in the colonies resistant to rifampicin, nalidixic acid and triclosan are shown below:

### Rifampicin

Clone MO#161 contains two mutations in rpoB, at amino acid 95, CCG(Pro) to CTG(Leu), and at amino acid 531, TCC(Ser) to TTC(Phe). The latter is a mutation known for rifampicin resistance (J. Mol. Biol. (1988) 202, 45-58).

Clone MO#166 contains ssrS. It has been found that over-expression of ssrS affords Rifampicin resistance.

### Nalidixic Acid

Clones MO#167 and MO#168 both contain one mutation in gyrA at amino acid 83, TCG(Ser) to TTG(Leu). This is a mutation known to afford nalidixic acid resistance (Drugs 45 (Suppl. 3): 15-23, 1993).

Clones MO#169 and MO#170 both contain one mutation in gyrA at amino acid 83, TCG(Ser) to TTG(Leu). This mutation is also known to afford nalidixic acid resistance (Drugs 45 (Suppl. 3): 15-23, 1993).

### Triclosan

Clones MO#162 and MO#163 both contain one mutation in fabI at amino acid 93, GGT(Gly) to GTT(Val). This is a novel mutation.

Clones MO#164 and MO#165 contain two mutations in fabI. The first is at amino acid 93, GGT(Gly) to AGT(Ser). This is the same novel amino acid swap as above. The second is at amino acid 120, AGC(Ser) to AAC(Asn). This mutation may be unrelated to triclosan resistance. To validate its relevance, it should be examined independently of the Gly -->Ser change.

### Example 7

### Genome-wide E. coli ORFmer Overexpression Rescue Assay

A Genome-wide Transformation Selection for the gene targets of the following antibiotics with known molecular targets was performed: triclosan (fabI) (Heath R. J. et. al., J. Biol. Chem. 1998 273:30316-20),trimethoprim(folA) (Neuwald et al., Gene, 1993, 125:69-73), D-cycloserine (ddlA), Belanger, et al. J. Bacteriol.,.2000 182:6854-6;Neuhaus, et al., Biochemistry, 1964,3:471-80, and phosphomycin (murA); Horii et al., Antimicrobial Agents & Chemo. 1999, 43:789-93. Over-expression rescue was observed for each of the antibiotics tested. For 3 of the 4 antibiotics tested, the expected gene target was responsible for the OER phenotype.

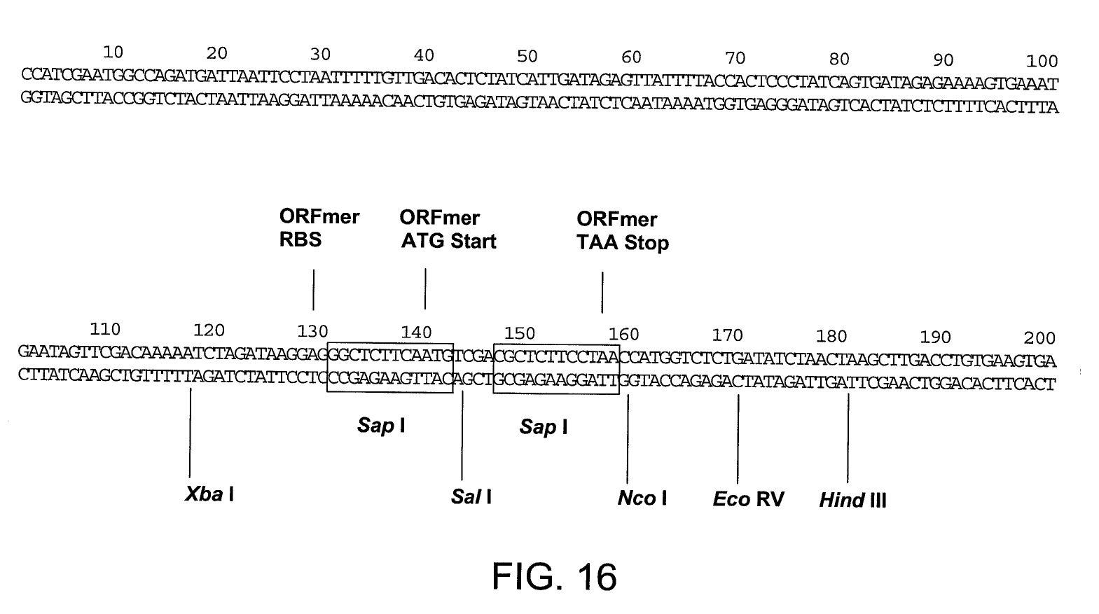

FIG. 16 shows the nucleotide sequence (nucleotides 1-200) of the ORFmer cloning site of pHO/0003, showing location of introduced Sap I restiction sites, ORFmer ribosome binding site (RBS), ORFmer ATG translation initiation codon, ORFmer TAA stop codon and other restriction sites.

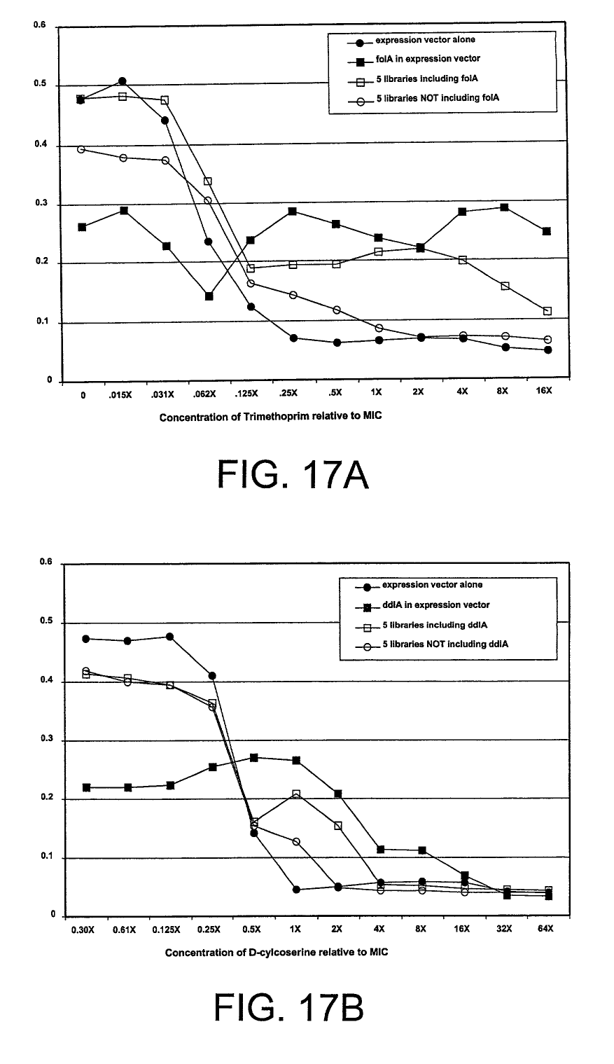

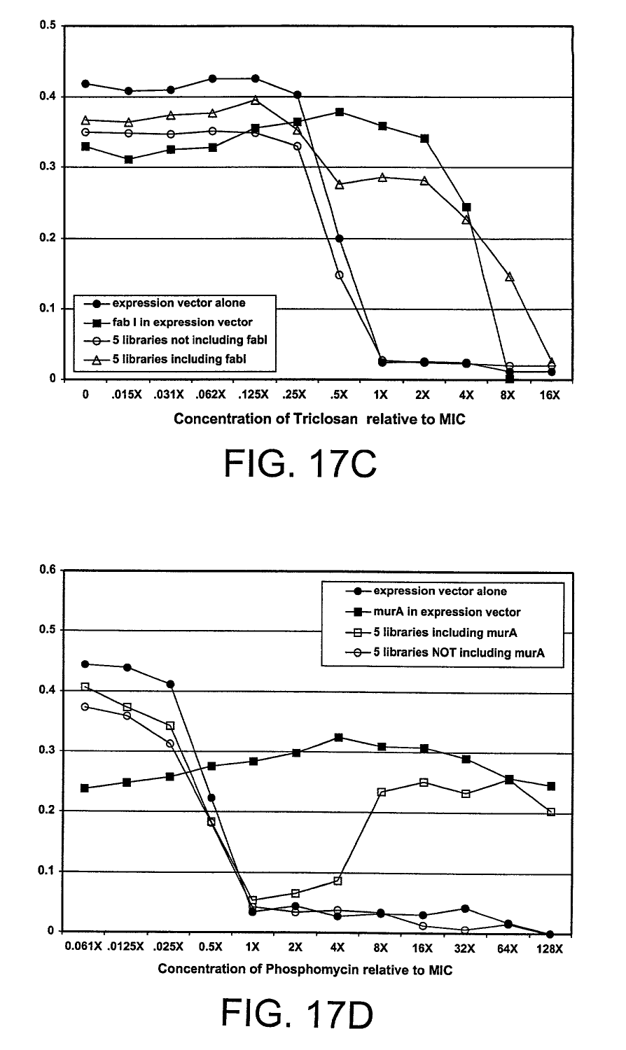

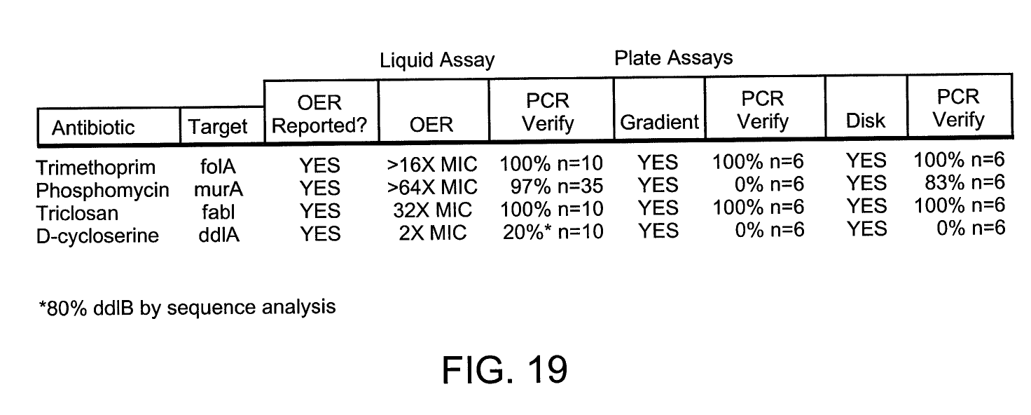

FIG. 17 shows results of an OverexpressionRescue (OER) Assay performed in a liquid microtiter format using the following antibiotics with known molecular targets (shown in parentheses): Trimethoprim (folA), D-cycloserine (ddlA), Triclosan (fabI), and Phosphomycin (murA). OER was observed in samples containing up to 500 E. coli genes when the known target gene of the antibiotic was present. Individual clones were isolated from wells exhibiting OER to verify by a PCR assay that the expected gene was responsible for the OER phenotype. Panel A. OER assay using Trimethoprim (10 ng/ml anhydrous tetracycline (a-tet), Panel B. OER assay using D-cycloserine (100 ng/ml a-tet), Panel C. OER assay using Triclosan (100 ng/ml a-tet), Panel D. OER assay using Phosphomycin (100 ng/ml a-tet). A summary of the results of the verification are shown in FIG. 19.

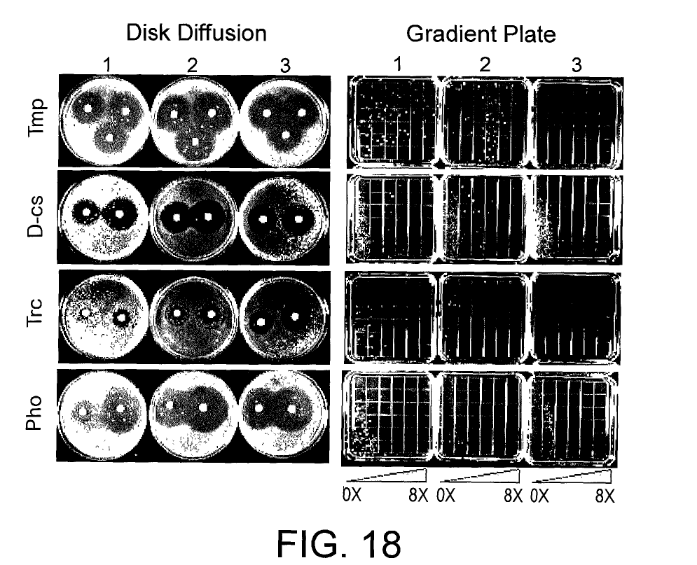

FIG. 18 shows the results of an Overexpression Rescue (OER) Assay performed in two plate formats using the following antibiotics with known molecular targets (shown in parentheses): Trimethoprim (folA), D-cycloserine (ddlA), Triclosan (fabI), and Phosphomycin (murA). The two plate formats were as follows: Disk Diffusion format and the Gradient plate format. The following inocula were plated: column 1—one expression library containing expected gene target, column 2—five expression libraries including expected gene target, column 3—five expression libraries excluding expected gene target. In the Disk diffusion plate format, the 3 inocula were plated on MHB plates containing ampicillin (100 mcg/ml) and anhydrous tetracycline (a-tet, 100 ng/ml). Two paper disks (6 mm diameter) containing 10× and 20×MIC of the test antiobiotic were placed on the surface of the plate. OER clones grew in the Zone of Inhibition produced by the antibiotic as it diffused from the disk into the agar. The Gradient plates were made from MHB agar containing a-tet (100 ng/ml) and contained a single-dimensional gradient of the test antibiotic (0 to 8×MIC). In both formats OER was observed in samples containing up to 500 E. coli genes only when the known target gene of the antibiotic was present. Individual OER clones were picked from plates to verify by a PCR assay that the expected gene was responsible for the OER phenotype. The results of the verification are shown in FIG. 19.

Materials. The bacterial strains used are listed in Table 7. The E. coli plasmids utilized are listed in Table 8. The oligonucleotide primers used are listed in Table 9. The media and chemicals utilized in the experiments are as follows. Mueller Hinton broth (Difco; Cat. No. 0757-17-6), trimethoprim lactate (Sigma; Cat. No. T-0667), phosphomycin(Sigma; Cat. No. P-5396), ampicillin (Sigma; Cat. No. A-9518), D-cycloserine (Sigma; Cat. No. C-6880), anhydrotetracycline(Clontech; Cat. No. 8633-1), SapI restriction enzyme (New England Biolabs; Cat. No. #569S), SalI restriction enzyme (New England Biolabs; Cat. No. #138S), QIAQuick PCR purification kit (Qiagen; Cat No. 28104), Rapid DNA Ligation Kit (Roche; Cat. No. 1 635 379), Subcloning Efficiency DH5α Competent Cells (Gibco BRL; Cat. No. 18265-017), E.coli ORFmer Primer pairs (Sigma-GenoSys; Cat. No. ECPL0001 through ECPL0045), E. coli Universal Adaptamer Primer Pair (Sigma-GenoSys; Cat. No. ECAP0001), calf intestinal alkaline phosphatase (New England Biolabs; Cat. No. #290S), SeaPlaque agarose (FMC Bioproducts; Cat. No. 50100), QIAquick Gel Extraction Kit (Qiagen; Cat. No.28704) phenol/chloroform (Ambion; Cat. No. 9732), T4 DNA Ligase (New England Biolabs; Cat. No. #202S) ElectroMAX DH5α-E_Cells (Gibco BRL; Cat. No. 11319-019)‘Q’ Tray (Genetix LTD; Cat. No. X6021) QIAfilter Plasmid Maxi Kit (Qiagen; Cat. No. 12263), ECL direct Nucleic Acid Labeling and Detection System (AmershamPharmacia; Cat. No. RPN3000), 96-well microtiterplates (Nunc; Plates—Cat. No. 262162; Lids—Cat. No. 264122), PCR SuperMix High Fidelity (Gibco BRL; Cat. No. 10790-020).

Methods. Modification of an expression vector for ORFmer cloning. PCR products produced by amplifying E.coli chromosomal DNA with “ORFmer” primers from Sigma GenoSys had been designed to include sites for the restriction enzyme SapI. The pASKBA5 (Genosys) expression plasmid was modified to allow cloning of ORFmer PCR products digested with Sap I under the control of the PttA promoter. This was achieved using primers F and L (See Table 9) in the PCR to amplify the plasmid pASKBA5, purifying the product with the QIAquick PCR purification kit, digesting with SalI restriction enzyme, and ligating with Roche rapid ligation kit. The ligation was transformed into DH5α_subcloning-grade competent cells, and plasmids from transformants were sequenced to confirm the introduction of SapI sites as expected. This modified plasmid was named pHO/0003. The sequence of the modified ORFmer cloning site of pHO/0003 is shown in FIG. A. The ORFmer PCR product encoding the E.coli lacZ gene was cloned into pHO/0003 and inducer-dependent expression was confirmed using the Miller β-galactosidase assay (Miller, J. H. 1972. EXPERIMENTS IN MOLECULAR GENETICS. Cold Spring Harbor Laboratory).

TABLE 7
Bacterial strains used in GARIT E. coli ORFmer OER Assay

| Strain | species | genotype | source | Reference/cat # |
|---|---|---|---|---|
| KM354 | E. coli K-12 | argE3 his-4 leuB6 proA2 thr-1 ara-14 galK2 | Keenan Murphy | Murphy, 1998, J. |
| | | lacY1 mtl-1 xyl-5 thi-1 rpsL31 tsx-33 supE44 recJ | | Bacteriol. 180: 2063- |
| | | | | 71 |
| DH10B | E. coli K-12 | F⁻mcrA Δ(mrr-hsdMRS-mcrBC) F80dlacZ Δ M15 | Life Technologies Inc. | LTI‡ catalog, 1999 |
| | | Δ lacX74 deoR recA1 endA1 araD139 Δ(ara, | (Gaithersburg, MD) | |
| | | leu)7679 galU galK1⁻ rpsL nupG | | |
| WO-153 | E. coli K-12 | KM354, asmB1, Δ tolC::FRT-kan-FRT | Genome Therapeutics | |
| DH5α-E | E. coli K-12 | F-φ80dlacZΔM15 Δ(lacZYA-argF) U169 deoR | Gibco BRL | Donahue, R.A. Jr. and |
| | | recA1 endA1 hsdR17(r_{K}-m_{K}+) gal-phoA supE44 | | Bloom, F.R. (1998) |

TABLE 8
Plasmids used in GARIT E. coli ORFmer OER Assay

| Plasmid | Description | Source | Reference/cat # |
|---|---|---|---|
| pHO/0003 | Expression vector with SapI restriction | Genome Therapeutics Corporation | |
| | sites. Ap^{R} P_{TET}promoter | | |
| pASKBA5 | Expression vector. Ap^{R} P_{TET}promoter | Sigma-GenoSys | Cat. No. PEVC0005 |
| pBAD/Myc-HisB | Expression vector. Ap^{R} arabinose P_{BAD} | Invitrogen; | Cat. No. V440-01 |
| | promoter | | |

TABLE 9
Oligonucleotide primers used in GARIT E. coli ORFmer OER Assay

| Primer name | Sequence (5′∪3′) | Source | Description |
|---|---|---|---|
| FolA | GCGCATCAGCATCGTGGAAT | Gibco BRL | Gene specific primer |
| MurA | AGTGCGGCAAAAAGGATAGG | Gibco BRL | Gene specific primer |
| FabI | AAAGGGTCAGCAGGGCAGAA | Gibco BRL | Gene specific primer |
| DdlA | CGCGTCAGGGTAATAAATGG | Gibco BRL | Gene specific primer |
| Nest One | GCAGGGTCGGAACAGGAGAG | Gibco BRL | Plasmid specific primer |
| F | Gtcgtcgt-gtcgac-gctcttcctaaccatg-gtctctgatatctaacta | Gibco BRL | Vector modification primer |
| L | Acgacgac-gtcgac-att-gaagagccctcctta-tctagatttttgtcga | Gibco BRL | Vector modification primer |

PCR Amplification of E. coli ORFmers. ORFmer PCR products were amplified from E.coli MG1655 chromosomal DNA according to product instructions from Sigma-GenoSys. Following primary amplification, ORFmer PCR products were re-amplified using the E.coli Universal Adaptamer Primer Pair to add extra bases to the ends of the PCR products allowing more efficient cleavage with SapI restriction enzyme.

Expression Library Construction. Expression libraries were contructed as follows. The expression plasmid pHO/0003 was digested with Sap I restriction enzyme, dephosphorylated by treatment with calf intestinal phosphatase (CIP) according to manufacturer's protocol, purified by size fractionation on a 1% SeaPlaque agarose gel, and extracted from agarose using QIAquick Gel Extraction Kit.

ORFmer PCR products were pooled in groups of 96 genes, corresponding to the plates in which the ORFmer PCR primers were provided by Sigma GenoSys. 10 ul of each 50 μl PCR reaction in a 96 well PCR plate was pooled, extracted with phenol/chloroform and precipitated with ethanol as described in MOLECULAR CLONING: A LABORATORY MANUAL, vol. 1-3 (Cold Spring Harbor Laboratory Press, 1989), digested with SapI restriction enzyme, and purified using the QIAquick PCR Purification Kit.

Pooled PCR product DNA was ligated overnight to pHO/0003 DNA using T4 DNA ligase from New England Biolabs. Ligation reactions were electroporated into ElectroMAX DH5α cells using a Gibco BRL Cell Porator Electroporation System according to manufacturer's protocol and plated on large 22 cm square LB agar plates containing ampicillin at 100 μg/ml. Approximately 50,000 colonies were routinely observed on each plate following transformation. Colonies growing on transformation plates were collected by adding 25 ml LB media to the plate and scraping colonies with a disposable bacteria spreader. Plasmid DNA was extracted from the collected cells using the Qiagen Plasmid Maxi Kit.

Plasmid library DNA was analyzed to determine the level of gene representation attained. Labeled plasmid library DNA was hybridized to ORFmer PCR products on a Southern blot using the ECL Direct Nucleic Acid Labeling and Detection System according to manufacturer's protocol. Typically, 90% of the genes in a library could be detected by Southern blot.

Over expression Rescue Assays. Over expression assays were conducted with the E. coli strain WO-153 described in Table 7. Construction of WO-0153. Strain WO-0153 carries mutations in two genes, lpxC and tolC, that result in increased sensitivity to chemical compounds that are normally blocked by the outer membrane of E. coli. The asmB1 mutation is a G to A transition at position 628 in the 1pxC gene. The phenotype of the asmB1 mutation is increased sensitivity to antibiotics that are normally blocked by the outer membrane of E. coli. The tolC gene encodes an outer membrane protein that acts as a channel for the acrAB efflux pumps (See for example, Fralick, J. 1996. J. Bacteriol. 178:5801-5805). In addition, tolC mutants exhibit increased permeability of the outer membrane. WO-0153 was constructed as follows. The asmB1 mutation was introduced into a DNA fragment containing the 1pxC gene and 1 kb of the 5′ end of the secA gene by site directed mutagenesis of the 1pxC gene using standard methods. A kan^{R} resistance cassette flanked by FRT sites was introduced 60 bp downstream of the 1pxC gene. The linear DNA construct was introduced into electrocompetent and recombinogenic E. coli KM354 (pTP223) as described by Murphy (See for example, Murphy, 1998, J. Bacteriol. 180: 2063-71). Transformants were selected on LB agar containing kanamycin (35 mcg/ml) and screened for sensitivity to novobiocin (20 mcg/ml). The presence of the asmB1 mutation in clones with the expected phenotype was verified by DNA sequence analysis. The kanR gene flanked by FRT sites was then excised from the 1pxC-secA intergenic region using a previously described method (See for example, Cherepanove, P. P, and Wackernagel, W. 1995. Gene 158: 9-14.). The tolC gene was deleted from KM354, asmB1 (pTP223 using the methods described in Example 1, which resulted in replacement of the tolC structural gene has been replaced by a kanR cassette. Transformants were selected on LB agar containing kanamycin (35 mcg/ml). Clones were screened for the presence of the tolC deletion using a PCR assay. WO-153 was cured of pTP223 by overnight growth in the absence of selection for tetR, plating on non-selective media, and screening for tet^{S} clones. Descriptions of these strain constructions may be found for example in Kloser, A. W., et al., (1996) J. Bacteriol. 178:5138-5143; Fralick, J. (1996). J. Bacteriol. 178:5801-5805; Fralick, J. A., et al. (1994) J. Bacteriol. 176:6404-6406; and Cherepanove, P. P, et al., (1995) Gene 158:9-14. Mutations in this strain confer increased sensitivity to a broad range of antibiotics that are normally excluded from the cell by the outer membrane. Plasmid library DNA was transformed into WO-153 by electroporation as described above. Cells were collected from transformation plates by scraping and were equalized to an optical density of 1.53 on a Beckman DU650 spectrophotometer(5×10⁻⁸ cfu/ml). Groups of five libraries were mixed and frozen in LB media plus 10% (vol/vol) glycerol by immersion in a dry-ice/ethanol bath for 5 minutes.

Over-expression assays were performed in liquid media in a microtiter format, in a gradient plate format, or in a disk-diffusion format. The protocol used for the microtiter format was similar to the protocol described above for the MIC assay. Aliquots of 5 library pools spanning the genome were thawed at room temperature and 200 ul of each was inoculated into its own 20 ml of Mueller Hinton media containing ampicillin at 200 ug/ml and anhydrous tetracycline inducer as appropriate. Typically, replicates were performed using a number of final inducer concentrations, ranging from 0 to 100 ng/ml. Following an adequate mixing, 50 ul of inoculated media was mixed with 50 ul of the test antibiotic in Nunc brand 96-well microtiterplates. Typically, each group of five libraries was mixed with a range of concentrations of antibiotic spanning the minimum inhibitory concentration (MIC) of the antibiotic. Well conditions of a typical assay are usually organized in rows containing various concentrations of the test compound such as 0, 0.25×, 0.5×, 1×, 2×, 4×, 8×, and 16×MIC. Columns may contain 10 different groups of libraries as well as null vector and no innoculum controls. After mixing inoculum plus media with antibiotic, well contents were mixed by pipetting and incubated at 37° C. for 16 to 20 hours. Optical density at 600 nm was measured using a Molecular Devices SpectraMAX 250 spectrophotometer. Library groups that showed growth at antibiotic concentration higher than the vector control and the majority of the other library groups were considered to exhibit rescue. Contents of wells showing rescue were transferred to LB agar plates containing ampicillin at 100 ug/ml, and were spread to obtain single colonies. Alternately, it is possible to maintain selection for the antibiotic being assayed by including it in the agar plates at this point.

OER Assay using Disk diffusion and Gradient Plate Assay formats. In the Disk diffusion plate format, the 3 inocula (see below) were plated on MHB plates containing ampicillin (100 mcg/ml) and anhydrous tetracycline (a-tet, 100 ng/ml). Two paper disks (6 mm diameter) containing 10×and 20×MIC of the test antiobiotic were placed on the surface of the plate. OER clones grew in the Zone of Inhibition produced by the antibiotic as it diffused from the disk into the agar. in the gradient plate assay format, the gradient plates were made from MHB agar containing a-tet (100 ng/ml) and contained a single-dimensional gradient of the test antibiotic (0 to 8×MIC). The test antibiotic gradient (0×-8×MIC) was formed as follows. 20 ml of MHB containing ampicillin (100 mcg/ml), a-tet (100 ng/ml) and the test antibiotic at a concentration of 8×MIC was pipetted into a 100×100×15 mm square Petri dish (Simport, Quebec, Canada). One end of the Petri dish was placed on a 1 ml disposable pipet, raising its level by approximately 5 mm. After the agar hardened the plate was placed on a level surface and 20 ml of MHB agar containing ampicillin (100 ug/ml) and a-tet (100 ng/ml) was pipetted over the bottom layer containing the test antibiotic. The gradient of test antibiotic is formed by diffusion of the antibiotic from the bottom agar layer into the top layer. The following inocula were prepared and plated onto the disk diffusion and gradient plates: column 1—one expression library containing expected gene target (5×10⁵ cfu), column 2—five expression libraries including the expected gene target (5×10⁵ cif/library), column 3—five expression libraries excluding the expected gene target target (5×10⁵ cfu/library).

PCR assay to verify identity of OER clones. In colony PCR, single colonies were picked from agar plates with a pipette and suspended in 20 ul water. Two microliters of this suspension was added to a PCR reaction prepared according to the protocol for PCR Supermix High-Fidelity (Gibco BRL). All reactions included the vector-specific primer Nest One and a gene specific primer named for the target to be amplified (see Table 9). Reactions amplifying a fragment of the expected size were considered to indicate that the expected gene was cloned in the examined plasmid. Confirmation is accomplished by determining the DNA sequence of the PCR product.

FIG. 19 shows results of PCR verification of clones exhibiting OER in three assay formats for antibiotics with known molecular targets. OER was observed for all of the antibiotics tested. The expected target gene was identifed for clones that exhibited OER for the following antibiotics in the liquid microtiter assay: trimethoprim, phosphomycin, and triclosan. 80% of OER clones isolated for D-cycloserine were not the expected ddlA gene, however, subsequent sequence analyses of these clones revealed that these clones were the closely related ddlB gene. OER clones were also isolated using the two plate assay format. The expected clone was isolated for the antibiotics: trimethoprim, phosphomycin and triclosan.

Although the present invention has been described in detail with reference to examples above, it is understood that various modifications can be made without departing from the spirit of the invention, and would be readily known to the skilled artisan. All cited patents and publications referred to in this application are herein incorporated by reference in their entirety.

What is claimed is:

1. A method for identifying one or more molecular targets of a cell growth inhibiting compound comprising the steps of:
identifying a compound or composition which inhibits growth in a first population of cells;
performing a plurality of target prediction processes using the cell growth inhibiting compound or composition to identify one or more genes or gene products that are modulated in the presence of the cell growth inhibiting compound or composition,
comparing the genes or gene products predicted by each of the plurality of target prediction processes with the genes or gene products identified by each of the other target prediction processes; and,
selecting one or more than one gene or gene product from the one or more than one gene or gene product identified by one or more than one of the plurality of target prediction processes, wherein the selection is based on the comparison, and
identifying the one or more than one selected gene or gene product as a molecular target of the cell growth inhibiting compound or composition.

2. The method of claim 1, wherein the molecular target is a mRNA or a protein.

3. The method of claim 1, wherein the target prediction processes can be separately performed.

4. The method of claim 1, wherein the plurality of target prediction process comprise a transformation selection process.

5. The method of claim 1, wherein the plurality of target prediction processes comprises a gene expression profiling process.

6. The method of claim 1, wherein the plurality of target prediction processes comprises a mutation to resistance process.

7. The method of claim 1, wherein the step of comparing the genes or gene products identified by each of the plurality of target prediction processes with the genes or gene products identified by each of the other target prediction processes comprises the step of:
determining whether at least two of the target prediction processes identify a common gene or gene product.

8. The method of claim 1, wherein the step of selecting one or more genes or gene products from among the one or more than one gene or gene product predicted by one or more than one of the plurality of target prediction processes comprises the step of:
selecting a gene or gene product if at least two of the target prediction processes independently identify said gene or gene product as being functionally modulated by the presence of the inhibitory compound.

9. The method of claim 1 further comprising the step of:
assigning a weighting factor to each of the target prediction processes.

10. The method of claim 9, wherein the step of selecting one or more genes or gene products from one or more than one gene or gene product identified by one or more than one of the plurality of target prediction processes is a function of the weighting factor assigned to each of the target prediction processes.

11. The method of claim 1 further comprising the step of:
determining whether any gene identified by any of the plurality of target prediction processes encodes an efflux pump or any gene product identified by any of the plurality of target prediction processes serves as an efflux pump.

12. The method of claim 11 further comprising the step of:
disregarding any gene identified by any of the plurality of target prediction processes which encodes an efflux pump or any gene product identified by any of the plurality of target prediction processes which serves as an efflux pump.

13. The method of claim 1 further comprising the step of.
determining whether any gene identified by any of the plurality of target prediction processes encodes a drug modification enzyme or any gene product identified by any of the plurality of target prediction processes serves as a drug modification enzyme.

14. The method of claim 13 further comprising the step of.
disregarding any gene identified by any of the plurality of target prediction processes which encodes a drug modification enzyme or any gene product identified by any of the plurality of target prediction processes which serves as a drug modification enzyme.

15. The method of claim 1 further comprising the steps of:
controlling expression of a selected gene, or a gene corresponding to a selected gene product, in a second cell population; and
validating the selection of said gene or gene product based on the characteristics of the second population of cells.

16. A method of identifying one or more than one molecular target of a cell growth inhibitory compound comprising the steps of:
performing a plurality of prediction processes using the cell growth inhibitory compound wherein at least one of the processes is selected from transformation selection, gene expression profiling, proteomic profiling, metabolic profiling, and mutation to resistance, and thereby identifying one or more than one gene or gene product using a first population of cells, wherein the gene or gene product is functionally modulated in the presence of the inhibitory compound;
selecting, from among the one or more identified genes or gene products, a first gene or gene product;
controlling the expression of the selected first gene, or a gene associated with the selected first gene product, through a regulatable promoter in a second population of cells; and
determining whether the selected first gene product, or a gene product associated with the selected first gene, is a valid molecular target of the inhibitory compound based on characteristics of the second population of cells.

17. The method of claim 16, wherein the target prediction processes may be separately performed

18. The method of claim 17, wherein the plurality of target prediction processes comprises a transformation selection process.

19. The method of claim 17, wherein the plurality of target prediction processes comprises a gene expression profiling process.

20. The method of claim 17, wherein the plurality of target prediction processes comprises a mutation to resistance process.

21. The method of claim 16 further comprising the step of:
determining whether at least two of the target prediction processes identify the same gene or gene product.

22. The method of claim 21, wherein said step of selecting, from among the identified one or more than one genes or gene products, a first gene or gene product comprises the step of:
selecting a gene or gene product that has been identified by at least two of the target prediction processes.

23. The method of claim 16 further comprising the step of:
determining whether the second population of cells is hypersusceptible to the presence of the inhibitory compound when the first selected gene is underexpressed.

24. The method of claim 23, wherein the step of determining whether the selected first gene product, or a gene product associated with the selected first gene, is a valid molecular target of the inhibitory compound comprises the step of:
validating the selected first gene product, or a gene product associated with the selected first gene if it is determined that the second population of cells is hypersusceptible to the presence of the inhibitory compound when the first selected gene or the gene associated with the selected first gene product is underexpressed.

25. The method of claim 16 further comprising the step of:
determining whether a gene expression or proteomic profile of cellular associated with the second population of cells is significantly different from a profile associated with the first population of cells when exposed to sub-lethal doses of the inhibitory compound.

26. The method of claim 25, wherein the step of determining whether the selected first gene product, or a gene product associated with the selected first gene, is a valid molecular target of the inhibitory compound comprises the step of:
validating the first gene product, or the gene product associated with the selected first gene, if it is determined that the profile associated with the second population of cells is not significantly different from the profile associated with the first population of cells when exposed to sub-lethal doses of the inhibitory compound.

27. The method of claim 16 further comprising the step of:
selecting, from among the one or more than one gene or gene product that are functionally modulated by the presence of the inhibitory compound, a second gene or gene product.

28. The method of claim 27 further comprising the steps of:
in a third population of cells, controlling the expression of the selected second gene, or a gene associated with the selected second gene product, through a regulatable promoter; and
determining whether the selected second gene product, or the gene product associated with the selected second gene, is a valid target of the inhibitory compound based on characteristics associated with the third population of cells.

29. The method of claim 28 further comprising the steps of:
in a fourth population of cells, controlling the expression of the selected first gene, or the gene associated with the selected first gene product, and controlling the expression of the selected second gene, or the gene associated with the selected second gene product, through regulatable promoters; and
determining whether the selected first gene product, or the gene product associated with the selected first gene, and the selected second gene product, or the gene product associated with the selected second gene, are valid co-targets of the inhibitory compound based on characteristics associated with the fourth population of cells.

30. The method of claim 1, wherein the cells are selected from the group organisms consisting of a bacterium, a fungus, an ameba, and a mycoplasma.

31. The method of claim 30, wherein the organism is a mycoplasma selected from the group consisting of: M. pneumoniae, M. fermentans, M. hominis and U. urealyticum.

32. The method of claim 30, wherein the organism is a fungus selected from the group consisting of: Histoplasma capsulatum, Coccidioides immitis, Paracoccidioides brasiliensis, Blastomyces dermatitidis, Cryptococcus neoformans, Candida albicans, Candida tropicalis, Candida parapsilosis, Candida guilliermondii, Candida glabrata, Candida krusei, Candida granuloma, Aspergillus fumigatus, Aspergillus flavus and Aspergillus niger.

33. The method of claim 30, wherein the organism is a protozoa selected from the group consisting of: Entamoeba histolytica, Naegleria fowleri, Plasmodium vivax, Plasmodium ovale, Plasmodium malariae, Plasmodium falciparum, Babesi microti, Babesi divergens, Leishmania chagasi, Leishmania mexicana, Leishmania amazonensis, Leishmania braziliensis, Leishmania guyanensis, Leishmania panamensis, Leishmania peruviana, Leishmania lainsoni, Leishmania naiffi, Leishmania columbiensis, and Trypanosoma cruzi.

34. The method of claim 30, wherein the organism is a bacterium is gram-positive or gram-negative.

35. The method of claim 34, wherein the bacterium is a gram-positive bacterium selected from the list consisting of: bacillaceae, micrococcaceae and peptococcaceae.

36. The method of claim 34, wherein the bacterium is a gram-negative bacterium selected from the list consisting of: acetobacteriaceae, alcaligenaceae, bacterioidaceae, chromatiaceae, enterobacteriaceae, legionellaceae, neisseriaceae, nitrobacteriaceae, pseudomonadaceae, rhizobiaceae, rickettsiaceae, spirochaetaceae and vibrionaceae.

37. The method of claim 35, wherein the gram-positive bacterium is bacillaceae and is selected from the group consisting of: B. acidocaldarius, B. anthracis, B. cereus, B. fastidiosus, B. firmus, B. licheniformis, B. macerans, B. megaterium, B. pasteurii, B. polymyxa, B. sphaericus and B. subtilis.

38. The method of claim 37, wherein the bacillaceae is B. subtilis selected from the strain group consisting of derivatives of type strain 168, derivatives of strain W23, strain niger and strain natto.

39. The method of claim 36, wherein the gram-negative bacterium is chromatiaceae and is selected from the genus group consisting of: Amoebobacter, Chromatium, Lamprobacter, Lamprocystis, Thiocapsa, Thiocystis, Thiodictyon, Thiopedia and Thiospirillum.

40. The method of claim 36, wherein the gram-negative bacterium is enterobacteriaceae and is selected from the genus group consisting of: Escherichia, Salmonella and Shigella.

41. The method of claim 40, wherein the enterobacteriaceae is Escherichia and is selected from the group consisting of: E. coli, E. blattae, E. fergusonii, E. hermani and E. vuneris.

42. The method of claim 41, wherein the enterobacteriaceae is E. coli and the strain of E. coli is K-12 selected from the group consisting of MC1061, KM354, KM29, DH5aPRO and DH10B; or) or the strain of E. colt is B strain selected from the group consisting of BL21 and BL21PRO).

43. The method of claim 40, wherein the enterobacteriaceae is Salmonella and is selected from the group consisting of: S. enterica, S. salamae, S. arizonae, S. diarizonae, S. houtenae and S. bongori.

44. The method of claim 36, wherein the gram-negative bacterium is legionellaceae and is L. pneumophila.

45. The method of claim 36, wherein the gram-negative bacterium is neisseriaceae and is selected from the species group consisting of: N. cinerea, N. gonorrhoeae, N. gonorrhoeae subsp. kochii, N. lactamica, N. meningitidis, N. polysaccharea mucosa, N. sicca, N. subflava, N. flavescens, N. caviae, N. cuniculi and N. ovis.

46. The method of claim 36, wherein the gram-negative bacterium is pseudomonadaceae and is selected from the genus group consisting of: Pseudomonas, Xanthomonas, Zoogloea and Fraturia.

47. The method of claim 46, wherein the gram-negative bacterium is Pseudomonas selected from the species group consisting of: P. aeruginosa, P. cepacia, P. chlororaphis, P. cichori, P. fluorescens, P. mallei, P. pseudomallei, P. putida, P. solanacearum, P. stutzeri, P. syringae, and P. testosteroni.

48. The method of claim 36, wherein the gram-negative bacterium is spirochaetaceae and is selected from the group consisting of: Treponema denticola, T. hyodysenteriae, T. innocens, T. pallidum, T. pectinovorum, T. phagedensis, T. socranskii and T. vuncentii.

49. The method of claim 36, wherein the gram-negative bacterium is vibrionaceae and is selected from the group consisting of: V. cholerae, V. parahaemolyticus and V. vulnificus.

50. The method of claim 1, wherein the inhibitory compound is: an aminoglycoside, an amphenicol, an ansamycin, a β-lactam, a lincosamide, a macrolide, a polypeptide, a tetracycline, a 2,4-diaminopyrimidine, a nitrofuran, a quinolone, a quinolone analog, a sulfonamide, and a sulfone.

51. The method of claim 50, wherein the β-lactam is selected from the group consisting of: carbacephems, carbapenems, cephalosporins, cephamycins, cephamycins, monobactams, oxacephems and penicillins.

52. A resistance cassette used in the transformation selection step of claim 4, wherein the resistance cassette comprises an inducible inducible promoter selected from the group consisting of: P_{araBAD}, P_{rhaBAD}, P_{LtetO-1}, P_{lac/ara-1}, P_{lac}, P_{trc}, Pₜᵣₚ, IP_{L}, P_{tetA} for E. coli, P_{xyl-tetO1}, P_{spac}, P_{nisA} for B. subtilis, P_{araE} for B. subtilis, and P_{xylA} for B. subtilis.

53. A compound which regulates the activity of a gene or product thereof in a cell or microorganism identified by the method of claim 1.

54. The method of claim 1, wherein the molecular target established is a gene product encoded by a gene wherein the gene is an essential gene.

55. An essential gene identified by the method of claim 54.

56. A method for developing compounds for antibiotic, antimicrobial, or antifungal applications comprising the steps of:
a) screening one or more than one compound for inhibiting growth of a cellular population;
b) selecting an active compound identified in step a);
c) performing a plurality of processes for the purpose of predicting the molecular target of the selected active compound comprising at least two processes chosen from a transformation selection protocol, a hyper-susceptibility protocol; a gene expression profiling protocol, a mutation to resistance protocol, and a three-hybrid screen protocol;
e) scoring the results of the molecular target predicting processes of step c) for any gene or gene product predicted by the processes of step c);
d) analyzing the scored results of the molecular target predicting processes for the purpose of identifying one or more molecular targets associated with the active compound.

57. The method of claim 56, wherein the plurality of processes for the purpose of predicting the molecular target of the selected active compound comprise a transformation selection protocol, a gene expression profiling protocol, and a mutation to resistance protocol.

58. The method of claim 56, the method further comprising a step of:
e) validating the identified molecular target.

59. The method of claim 58, wherein the step of validating the one or more than one identified molecular target comprises performing an underexpression assay or performing a genomic or proteomic profiling assay or both.

60. The method of claim 56, wherein the scoring of the results of the molecular target predicting processes of step c) is quantitative, and wherein the scores are weighted by weighting factors according to each target predicting process.

61. The method of claim 60, wherein the weighting factors according to each target predicting process are such that mutation to resistance >transformation selection>yeast three-hybrid screen>gene-expressionprofile>metabolic profile>proteomic profile.
